(19) United States
(12) Patent Application Publication
(10) Pub. No.: US 2024/0417695 A1
(43) Pub. Date: Dec. 19, 2024

(54) ENDOTHELIAL CELL FACTORS AND METHODS THEREOF

(71) Applicant: THE CHILDREN'S MEDICAL CENTER CORPORATION, Boston, MA (US)

(72) Inventors: Leonard I. ZON, Brookline, MA (US); Elliott J. HAGEDORN, Jamaica Plain, MA (US)

(73) Assignee: THE CHILDREN'S MEDICAL CENTER CORPORATION, Boston, MA (US)

(21) Appl. No.: 18/824,457

(22) Filed: Sep. 4, 2024

Related U.S. Application Data
(62) Division of application No. 17/040,421, filed on Sep. 22, 2020.
(60) Provisional application No. 62/647,433, filed on Mar. 23, 2018.

(51) Int. Cl. C12N 5/071; A61K 35/44; C12N 15/90

(52) U.S. Cl. CPC C12N 5/069; A61K 35/44; C12N 15/907; C12N 2501/60; C12N 2510/00

(57) ABSTRACT

The technology described herein relates to compositions and methods of generating endothelial niche cells. Embodiments of the technology described herein comprise compositions, kits, vectors, and methods related to generating or engineering endothelial niche cells. One aspect comprises a method to generate/engineer endothelial niche cells, comprising expressing one or more transcription factors in an endothelial cell, wherein the one or more transcription factors are from the Ets family, the Sox family, and/or the Nuclear Hormone (NHR) family.

### CROSS-REFERENCE TO RELATED APPLICATIONS

This application is a divisional under 35 U.S.C. § 121 of co-pending U.S. application Ser. No. 17/040,421, filed Sep. 22, 2020, which is a 35 U.S.C. § 371 National Phase Entry Application of International Patent Application No. PCT/US2019/023637 filed on Mar. 22, 2019, which claims benefit under 35 U.S.C. § 119(e) of U.S. Provisional Application No. 62/647,433 filed Mar. 23, 2018, the contents of which are incorporated herein by reference in their entireties.

### GOVERNMENT SUPPORT

This invention was made with government support under grant number DK111790 and HL048801 awarded by the National Institutes of Health. The Government has certain rights in the invention.

### SEQUENCE LISTING

The instant application contains a Sequence Listing which has been submitted in XML format via Patent Center and is hereby incorporated by reference in its entirety. Said XML copy, created on Sep. 3, 2024, is named 701039-091810USD1_SL.xml and is 325,744 bytes in size.

### TECHNICAL FIELD

The technology described herein relates to compositions and methods of generating endothelial niche cells.

### BACKGROUND

Haematopoietic stem and progenitor cells (HSPCs) are a rare cell population capable of reconstituting the entire blood system after transplantation. As the functional unit of a bone marrow transplant, these cells offer a curative treatment for many blood and immune diseases. Unfortunately, transplantation is not a viable treatment option for many individuals, particularly those lacking an immune-matched donor. A long-term goal of haematological research has been to culture and expand HSPCs in vitro, for use in transplantation and/or genetic modification. While umbilical cord blood-derived HSPCs are somewhat amenable to in vitro expansion, maintaining and inducing self-renewal of adult-derived HSPCs, in the absence of niche signals, has proven challenging.

Strategies aimed at in vitro expansion have co-cultured HSPCs with supportive cells in an effort to recapitulate aspects of the microenvironment or ‘niche’ that supports HSPCs in vivo. In the adult bone marrow, multiple cell types are thought to collectively comprise the HSPC niche, with primary contributors being endothelial cells (ECs) and perivascular mesenchymal stromal cells. Different endothelial cell subtypes in the bone marrow can differentially regulate HSPC homeostasis. Arterial ECs (AECs) are less permeable and are believed to promote HSPC quiescence, while sinusoidal ECs (SECs) are leaky and support the differentiation and mobilization of HSPCs. In addition, during haematopoietic recovery after myelosuppression, ECs play a critical role in niche reconstruction and reconstitution of multi-lineage haematopoiesis. HSPCs can also be supported outside the bone marrow, during embryonic development and under stress conditions that induce extramedullary haematopoiesis in tissues such as the liver, spleen and skull. As in the bone marrow, ECs are thought to function as critical, core components of the HSPC niches in these tissues.

Researchers have focused on the development of in vitro cultures where HSPCs can be grown in the lab with other cells types that support the maintenance or expansion of the HSPCs for subsequent use in transplantation. To date, however, these in vitro cultures have been only modestly successful.

### SUMMARY

In studies of the vascular HSPC niche in the zebrafish embryo a combination of transcription factors (from the Ets, Sox and Nuclear Hormone families) that are normally expressed in the endogenous niche endothelial cells were studied. When human orthologs of these same transcription factors were ectopically expressed, ectopic vascular niches in the zebrafish embryo were generated, to which HSPCs are recruited and maintained.

As a step towards translating these findings into a clinical application, transcriptions factors (which initially were identified in the zebrafish studies) can be expressed in human endothelial cells to reprogram these cells into an HSPC niche-like identity. These niche endothelial cells can be used in co-cultures with HSPCs in order to expand HSPC numbers or extend culture times, for subsequent use in transplantation.

For example, the transcription factors known to bind Ets, Sox and Nuclear hormone motifs can be expressed in niche endothelial cells. In the Ets family these factors include etv2, fli1a and ets1; where the corresponding human factors are ETV2, FLI1 and ETS1. In the Sox family these transcription factors include sox18 and sox7; where the corresponding human factors are SOX7 and SOX18. In the Nuclear hormone family these transcription factors include rxraa and nr2f2; where the corresponding human factors are RXRA and NR2R2.

The present invention provides a method for making synthetic niche endothelial cells, to stimulate blood stem cells. Transcription factors include Ets family, etv2, fli1a, ets1; SOX family: sox18, sox7, and Nuclear hormone family: rxraa, nr2f2) and the corresponding human factors: ETV2, FLI1, ETS1, SOX7, SOX18, RXRA, and NR2F2.

The method comprises expressing transcription factors in endothelial cells (e.g., human) to reprogram these cells into an HSPC niche-like identity.

In another embodiment, the niche endothelial cells are used in co-cultures with HSPCs in order to expand HSPC numbers or extend culture times, for subsequent use in transplantation.

One aspect provides a method to generate/engineer endothelial niche cells, comprising expressing one or more transcription factors in an endothelial cell, wherein the one or more transcription factors are from the Ets family, the Sox family, and/or the Nuclear Hormone Receptor family.

Another aspect provides an engineered endothelial niche cell comprising one or more exogenous nucleic acid sequences encoding one or more transcription factors, wherein the one or more transcription factors are from the Ets family, the Sox family and/or the Nuclear Hormone Family.

Another aspect provides a composition comprising the engineered endothelial niche described herein.

Another aspect provides a method for culturing HSPCs, the method comprising culturing HSPCs in the presence of a population of engineered endothelial niche cells.

Another aspect provides a method of treating a subject, the method comprising, transplanting a composition comprising HSPCs and a population of engineered endothelial niche-cells into the subject.

Another aspect provides a method for enhancing engraftment of HSPCs, the method comprising administering a composition comprising HSPCs and a population of engineered endothelial niche cells to a subject in need thereof.

Another aspect provides a co-culture comprising engineered endothelial niche cells and HSPCs.

Another aspect provides a kit for culturing HSPCs, the kit comprising: a population of engineered endothelial niche cells, reagents and instructions for use thereof.

Another aspect provides a kit for generating engineered endothelial niche cells comprising: a vector(s) comprising one or more exogenous nucleic acid sequences encoding one or more transcription factors of the Ets family, the Sox family or the nuclear hormone family and instructions for use thereof.

Another aspect provides a method for generating an ectopic vascular niche, the method comprising: administering an engineered endothelial niche cell to a target site in a subject in need thereof.

Another aspect provides a method for extra medullary hematopoiesis, the method comprising transplanting engineered-niche endothelial cells into a subject at a location outside of the bone marrow (e.g., the forearm), thereby creating a synthetic niche.

Another aspect provides a vector comprising one or more exogenous nucleic acid sequences encoding one or more transcription factors of the Ets family, the Sox family or the nuclear hormone family operably linked to a promoter.

### BRIEF DESCRIPTION OF THE DRAWINGS

FIG. 1A-FIG. 1E is a series of images and graphs showing an endothelial expression signature in the fetal HSPC niche. (FIG. 1A) Schematic diagram illustrates the haematopoietic tissues of the zebrafish embryo (top) and the sectioning strategy used to perform RNA tomography (tomo-seq) on the CHT (bottom; double transgenic embryo carrying the HSPC markers cd41:GFP and runx1:mCherry is shown). (FIG. 1B) Schematic cross-section and (FIG. 1B, cont.) hierarchical clustering heat map reveal clusters of gene expression that correspond to distinct tissues along the dorsal-ventral axis of the zebrafish tail. (FIG. 1C) Schematic depicts strategy using kdrl:GFP transgenic embryos and FACS to isolate ECs from whole embryos for analysis by RNA-seq. (FIG. 1D) Individual tomo-seq expression traces are shown for pan-endothelial expressed genes (left) and CHT EC-enriched genes (right). (FIG. 1E) Images show whole mount in situ hybridization (WISH) for the pan-endothelial gene kdrl (top) and CHT EC-enriched genes identified by tomo-seq and tissue-specific RNA-seq. Arrows point to expression in dorsal vasculature and arrowheads point to expression in the CHT. Scale bars represent 250 μm unless noted otherwise.

FIG. 2A-FIG. 2C is a series of images and graphs showing endothelial niche-specific cis-regulatory elements. (FIG. 2A) Image and schematic depict the four cell populations that were isolated from mrc1a:GFP, kdrl:mCherry double positive embryos for analysis by ATAC-seq. (FIG. 2B) Gene tracks show regions of chromatin that were uniquely open in the mCherry⁺; GFP⁺ CHT EC fraction (boxes and arrows). (FIG. 2C) Images show embryos injected with CHT EC enhancer-GFP reporter constructs corresponding to the boxed regions in FIG. 2B. Arrowheads point to GFP expression in CHT ECs. Scale bars represent 250 μm unless noted otherwise.

FIG. 3A-FIG. 3F is a series of images and graphs showing that Ets, Sox and NHR binding sites are required for selective expression in niche ECs. (FIG. 3A) Gene tracks show a region of chromatin (box and lower arrow) upstream of mrc1a that is uniquely open in the double positive CHT EC fraction but not the other three cell populations. Bars denote the position of the 125 bp enhancer sequence and the 1.3 kb sequence used to generate the mrc1a:GFP reporter transgene. (FIG. 3B) Images show transient GFP expression in an F0 embryo injected with the 125 bp enhancer sequence coupled to a minimal promoter and GFP. (FIG. 3C) Images show an F0 embryo simultaneously injected with kdrl:mCherry and mrc1a 125 bp:GFP plasmids. (FIG. 3D) Images show an embryo expressing the stably integrated mrc1a 125 bp:GFP transgene. (FIG. 3E) Wild-type sequence of the 125 bp mrc1a enhancer is shown (see e.g., SEQ ID NO: 12), annotated highlighting the Ets, Sox and NHR binding motifs. Schematic depicts enhancer-reporter constructs in which each class of motif or control regions was targeted by mutation. X's denote the location of targeted sites. mp-GFP: mouse Beta-globin minimal promoter fused to GFP. (FIG. 3F) Graphs report the frequency of embryos with GFP expression in CHT ECs after injection with wild-type sequences or mutated variants of the mrc1a 125 bp (top) or sele 158 bp (bottom) enhancers. Data is normalized to the respective wild-type control for each experiment (44% (155/356) for the mrc1a 125 bp enhancer and 23% (176/775) for the sele 158 bp enhancer). Mean +/− standard error of the mean (s.e.m.), One-way ANOVA; **P<0.01, ***P<0.001. Scale bars represent 250 μm unless noted otherwise.

FIG. 4A-FIG. 4F is a series of images and graphs showing that the overexpression of defined factors induces ectopic vascular gene expression outside the CHT. (FIG. 4A) Schematic depicts the strategy used in transcription factor overexpression experiments. (FIG. 4B) Images show embryos that were injected with control DNA (left) or a pool of seven transcription factors (right) from the Ets, Sox and NHR families (FLI1, ETV2, ETS1, SOX7, Sox18, Nr2f2 and RXRA) and then stained by WISH for mrc1a (top) or sele (bottom). Arrows denote regions of ectopic expression and arrowheads point to normal domains of expression in all panels of FIG. 4A-FIG. 4E. (FIG. 4C) Images show mrc1a:GFP; kdrl:mCherry double transgenic embryos that were injected with the control DNA or the 7-factor pool. (FIG. 4D) Injection of a 3-factor pool containing ETV2, SOX7 and Nr2f2 results in ectopic expression of mrc1a:GFP (arrows). (FIG. 4E) Images show WISH for mrc1a in a control embryo (top) or after injection of a 3-factor pool containing ETV2, SOX7 and Nr2f2 (middle) or ETS1, SOX7 and Nr2f2 (bottom). (FIG. 4F) Graph reports quantification of the percentage of injected embryos that displayed ectopic mrc1a expression after transcription factor overexpression. Chi Square Test; **P<0.01, ***P<0.001. Scale bars represent 250 μm in FIG. 4B and FIG. 4E, and 100 μm in FIG. 4C and FIG. 4D.

FIG. 5A-FIG. 5E is a series of images and graphs showing that HSPCs localize to regions of ectopic niche endothelial gene expression. (FIG. 5A) Images show runx1:mCherry⁺ HSPCs localized outside the CHT within a dorsal ectopic region of mrc1a:GFP expression in an embryo injected with a pool of ETV2, SOX7 and Nr2f2 (top) or a pool of ETS1, SOX7 and Nr2f2 (bottom). Inset magnifications with gray scale images for each channel are shown at right. Arrows point to ectopic expression or localization while arrowheads point to normal expression or localization in all panels in FIG. 5A-FIG. 5E. (FIG. 5B) WISH for runx1 shows HSPC localization in a control (top) and 3-factor injected embryo (bottom). (FIG. 5C) ECs ectopically expressing mrc1a:GFP are associated with cxcl12a:DsRed2⁺ stromal cells, similar to ECs in the CHT. Asterisk denotes notochord expression of cxcl12a:DsRed. (FIG. 5D) Time-lapse series shows a runx1:mCherry⁺ HSPC initially arriving in the CHT and subsequently dividing. Time is shown as hh:mm. (FIG. 5E) Time-lapse series from a different embryo than in FIG. 5D shows runx1:mCherry⁺ HSPCs dividing and migrating away into circulation. Scale bars represent 100 μm in FIG. 5A-FIG. 5C and 30 μm in FIG. 5D-FIG. 5E.

FIG. 6A-FIG. 6B is a series of images and graphs showing a conserved endothelial expression signature in the HSPC niche. (FIG. 6A) Heat map shows the expression of the 29 CHT EC genes in the different cell populations that comprise the adult zebrafish kidney marrow. Spectral scale shows normalized expression between 0 (low) and 1 (high). (FIG. 6B) Heat map shows the expression of orthologs of the CHT EC genes in ECs from different organs of the mouse at different stages of development and postnatal transition to adulthood. Arrows denote haematopoietic tissues at the respective stage of development. Black bracket denotes genes enriched in fetal liver ECs at the E14-17 stages and then later in the adult bone marrow. Spectral scale reports z-scores. BM: Bone Marrow.

FIG. 7A-FIG. 7B is a series of images and graphs showing RNA tomography and niche-specific endothelial gene expression. (FIG. 7A) Graphs show tomo-seq expression traces for individual tissue-specific genes. Images showing whole mount in situ hybridization (WISH) for 35 CHT-enriched genes are available on the world wide web on zfin.org. (FIG. 7B) WISH validates the CHT-enriched expression (arrowheads) of CHT EC genes identified using a combination of tomo-seq and tissue-specific RNA-seq. Scale bars represent 250 μm unless noted otherwise.

FIG. 8A-FIG. 8E is a series of images and graphs showing that GFP reporter transgenes selectively label ECs in the HSPC niche. (FIG. 8A) Images show a double transgenic embryo carrying the pan-endothelial marker kdrl:mCherry and the mrc1a:GFP transgene, which is selectively expressed in CHT ECs. Magnification of boxed area is shown on the right. (FIG. 8B) Images show runx1:mCherry⁺ HSPCs directly interacting with mrc1a:GFP⁺ ECs within the CHT niche (arrows). Middle panel shows magnification of boxed area. Additional magnification (bottom) shows an HSPC in a pocket of mrc1a:GFP⁺ ECs. (FIG. 8C) cxcl12a:DsRed2⁺ stromal cells are closely associated with mrc1a:GFP⁺ ECs in the CHT. (FIG. 8D) Images show a double transgenic embryo carrying the pan-endothelial marker kdrl:mCherry and the sele:GFP transgene, which is selectively expressed in CHT ECs. Magnification of boxed area is shown on the right. (FIG. 8E) Images show runx1:mCherry+ HSPCs directly interacting with sele:GFP+ ECs within the CHT niche (arrows). Magnification of boxed area is shown on the right. Scale bars represent 250 μm in FIG. 8A and FIG. 8D, and 100 μm in FIG. 8B, FIG. 8C, and FIG. 8E.

FIG. 9A-FIG. 9C is a series of images and graphs showing a pan-endothelial regulatory elements and genome-wide motif enrichment analysis. (FIG. 9A) Gene tracks show regions of chromatin that were open in both the mCherry⁺ GFP⁺ (CHT EC) and mCherry⁺ GFP⁻ (non-CHT EC) populations (boxes and straight arrows). (FIG. 9B) Images show embryos injected with pan-endothelial enhancer-GFP reporter constructs corresponding to the boxed regions in FIG. 9A. Arrows point to GFP expression in non-CHT ECs and arrowheads point to expression in CHT ECs. (FIG. 9C) Images show the transcription factor binding motifs most enriched in CHT EC regions (top) or pan-endothelial regions (bottom). Scale bars represent 250 μm unless noted otherwise.

FIG. 10A-FIG. 10E is a series of images and graphs showing CHT endothelial cis-regulatory elements. (FIG. 10A) Graph reports the anatomical location of endothelial expression in F0 embryos that were injected with mrc1a 125 bp:GFP and kdrl:mCherry plasmids. (FIG. 10B) Gene tracks show a region of chromatin upstream of sele that was uniquely open in the double positive CHT EC fraction but not the other three cell populations (box and lower arrow). Bars denote the position of the 158 bp enhancer sequence and the 5.3 kb sequence used to generate the sele:GFP reporter transgene. (FIG. 10C) Images show transient F0 (top) and stable F2 expression (bottom) of the sele 158 bp:GFP construct. (FIG. 10D) Wild-type sequence of the 158 bp sele enhancer is shown (see e.g., SEQ ID NO: 13), annotated highlighting the Ets, Sox and NHR binding motifs (top). Schematic depicts sequence variants in which each class of motif or control regions were targeted by mutation. X's denote the location of targeted sites. mp-GFP: mouse Beta-globin minimal promoter fused to GFP. (FIG. 10E) Images show electophoretic mobility shift assays with recombinant Nr2f2-GST that was incubated with DNA sequences spanning the NHR motifs present in the 125 bp mrc1a (left two gels) or 158 bp sele (right gel) enhancer sequences. Arrows point to DNA:protein binding while arrowheads point to super-shifted DNA:protein complexes. Labeled DNA:protein complexes were outcompeted by unlabeled wild-type probe (lane 4) but not by probe in which the NHR motif was disrupted by mutation (arrows with asterisks). Scale bars represent 250 μm unless noted otherwise.

FIG. 11A-FIG. 11C is a series of images and graphs showing that transcription factor overexpression induces ectopic CHT endothelial program. (FIG. 11A) Images show WISH for mrc1a over the yolk ball in a control (left) and 7-factor injected embryo (right). Arrows point to ectopic expression and arrowheads point to normal domains of expression in all panels of FIG. 11A-FIG. 11C. (FIG. 11B) Images show ectopic expression of the mrc1a:GFP and kdrl:mCherry transgenes over the yolk extension in a 7-factor injected embryo. Magnification of the boxed area is shown at the bottom. (FIG. 11C) Images show WISH for sele, gpr182 and lgmn in control embryos (left) and embryos injected with a combination of ETV2, SOX7 and Nr2f2 (right). Scale bars in FIG. 11A-FIG. 11C represent 100 μm.

FIG. 12A-FIG. 12D is a series of images and graphs showing that CHT niche endothelial gene expression is induced ectopically by transcription factor overexpression. (FIG. 12A-FIG. 12B) Injection of ETV2 alone induces ectopic expression of the endogenous mrc1a gene (FIG. 12A) and the mrc1a:GFP transgene (FIG. 12B). Arrows point to ectopic expression and black arrowhead point to the normal domain of expression in all panels in FIG. 12A-FIG. 12D. (FIG. 12C) Injection of human ETV2 alone induces ectopic expression of zebrafish transcription factors, including sox7, sox 18, fli1a and etv2. (FIG. 12D) Injection of a 3-factor pool containing ETS1, SOX7 and Nr2f2 results in ectopic expression of mrc1a:GFP. Scale bar represents 250 μm in FIG. 12A and FIG. 12C and 50 μm in FIG. 12B and FIG. 12D.

FIG. 13A-FIG. 13B is a series of images and graphs showing niche endothelial transgene expression in adult kidney ECs. (FIG. 13A) Images show a segment of vasculature (white arrows) dissected from the kidney of a kdrl:mCherry, mrc1a:GFP double transgenic adult zebrafish. (FIG. 13B) Images show sequential sections through an adult kidney isolated from a sele:GFP transgenic fish. Sections were stained with H&E (left) and with an antibody against GFP (right). Black arrows point to GFP vascular endothelial cells. Scale bar represents 50 μm in FIG. 13A and 100 μm in FIG. 13B.

FIG. 14 is a schematic showing hematopoietic stem cell self-renewal and differentiation. LT-HSC indicates a long-term hematopoietic stem cell. CMP indicates a common myeloid progenitor. MEP indicates a megakaryocyte-erythroid progenitor. GMP indicates a granulocyte-macrophage progenitor. CLP indicates a common lymphoid progenitor.

FIG. 15A-FIG. 15B is a series of images showing visualization of niche colonization by HSPCs in vivo. FIG. 15A shows visualization of the dorsal aorta and caudal hematopoietic tissue. FIG. 15B shows an HSPC surrounded by 5 endothelial cells and attached to stromal cell.

FIG. 16 is a series of images showing the use of RNA tomography (tomo-seq) to examine gene expression in the HSPC niche.

FIG. 17 is a series of images and graphs showing how Tomo-seq+endothelial RNA-seq identified ˜20 genes selectively enriched in niche endothelial cells.

FIG. 18 is a series of images showing that sele and mrc1a promoter-GFP fusions label endothelial cells in the HSPC niche.

FIG. 19 is a series of images showing that specific ATAC-seq peaks near original 20 genes can drive expression in niche endothelial cells. 13 out of 19 cloned ATAC-seq peaks can drive GFP expression in CHT endothelial cells (when coupled to a minimal promoter).

FIG. 20 is an image showing the TF binding motifs most enriched in the open chromatin of niche endothelial cells. Motif enrichment analysis was performed on 6,710 unique ATAC-seq peaks using HOMER.

FIG. 21 is a series of images and graphs showing that Ets, Sox and NHR sites are required for niche expression of a 158 bp sele enhancer (see e.g., SEQ ID NO: 14).

FIG. 22 is a series of images and graphs showing that Ets, Sox and NHR motifs are required for niche expression of a 125 bp mrc1a enhancer (see e.g., SEQ ID NO: 15).

FIG. 23 is a series of images showing that mouse TFs can bind zebrafish sequences in vitro. The same mutations that disrupt enhancer: GFP expression abrogate TF binding.

FIG. 24 is an image showing that transcription factors known to bind Ets, Sox and NH motifs are expressed in niche endothelial cells.

FIG. 25 is a series of images showing the reprogramming of niche endothelial cells.

FIG. 26 is a series of images and graphs showing that 3 TF pool injections result in ectopic niche endothelial gene expression.

FIG. 27A-FIG. 27C is a series of images showing that ectopic vascular patches can recruit runx1⁺ HSPCs.

FIG. 28 is a series of images and heat maps showing that HSPC niche endothelial signature is also in adult marrow.

FIG. 29 is a heat map showing that a similar niche endothelial signature found in the mammalian fetal liver and bone marrow.

FIG. 30 is a diagram showing that RNA tomography of the HSPC niche led to the following data: ˜20 genes are selectively enriched in HSPC niche endothelial cells; ectopic expression of just 3 TFs can induce niche endothelial gene expression and recruit HSPCs; and Ets, Sox and Nuclear Hormone motifs are required for expression in niche endothelial cells.

### DETAILED DESCRIPTION

Embodiments of the technology described herein comprise compositions, kits, vectors, and methods related to generating or engineering endothelial niche cells. One aspect comprises a method to generate/engineer endothelial niche cells, comprising expressing one or more transcription factors in an endothelial cell, wherein the one or more transcription factors are from the Ets family, the Sox family, and/or the Nuclear Hormone Receptor (NHR) family.

In some embodiments, at least one transcription factor is selected from the Ets family. In some embodiments, at least one transcription factor is selected from the Sox family. In some embodiments, at least one transcription factor is selected from the NHR family.

In some embodiments, at least one transcription factor is selected from the Ets family, and at least one transcription factor is selected from the Sox family. In some embodiments, at least one transcription factor is selected from the Ets family, and at least one transcription factor is selected from the NHR family. In some embodiments, at least one transcription factor is selected from the Sox family, and at least one transcription factor is selected from the NHR family.

In some embodiments, at least one transcription factor is selected from the Ets family, at least one transcription factor is selected from the Sox family, and at least one transcription factor is selected from the NHR family. In some embodiments, at least one transcription factor is selected from the Ets family, at least one transcription factor is selected from the Sox family, or at least one transcription factor is selected from the NHR family.

### Ets Family

In some embodiments of any of the aspects, endothelial niche cells express transcription factors from the ETS family. The ETS (E26 transformation-specific or E-twenty-six) family is one of the largest families of transcription factors and is unique to animals. The ETS family is divided into 12 subfamilies: 1) ELF (e.g., ELF1, ELF2/NERF, ELF4/MEF); 2) ELG (e.g., GABPα, ELG); 3) ERG (e.g., ERG, FL1, FEV); 4) ERF (e.g., ERF/PE2, ETV3/PE1); 5) ESE (e.g., ELF3/ESE1/ESX, ELF5/ESE2, ESE3/EHF); 6) ETS (e.g., ETS1, ETS, POINTED); 7) PDEF (e.g., SPDEF/PDEF/PSE); 8) PEA3 (e.g., ETV4/PEA3/E1AF, ETV5/ERM, ETV1/ER81); 9) ERF71 (e.g., ETV2/ER71); 10) SPI (e.g., SPI1/PU.1, SPIB, SPIC); 11) TCF (e.g., ELK1, ELK4/SAP1, ELK3/NET/SAP2, LIN); 12) TEL (e.g., ETV6/TEL, ETV7/TEL2, YAN).

All ETS family members are identified through a highly conserved DNA binding domain, the ETS domain, which is a winged helix-turn-helix structure that binds to DNA sites with a central GGA (A/T) DNA sequence. DNA motifs for the Ets family can also comprise a central TTCCT sequence (e.g., on DNA strand complementary to the first motif). As well as DNA-binding functions, evidence suggests that the ETS domain is also involved in protein-protein interactions.

The ETS family is present throughout the body and is involved in a wide variety of functions including the regulation of cellular differentiation, cell cycle control, cell migration, cell proliferation, apoptosis (programmed cell death) and angiogenesis.

Non-limiting examples of members of the human Ets family that are relevant to endothelial niche cells comprise ETV2, FLI1, and ETS1. The corresponding factors in zebrafish comprise etv2, fli1, and ets1.

ETV2 can also be referred to herein as ETS Variant 2, ETS Translocation Variant 2, Ets-Related Protein 71, Ets Variant Gene 2, ETSRP71, or ER71.

Friend leukemia integration 1 transcription factor (FLI1), also known as transcription factor ERGB, is a protein that in humans is encoded by the FLI1 gene. FLI1 can also be referred to herein as Fli-1 Proto-Oncogene, ETS Transcription Factor, Friend Leukemia Integration 1 Transcription Factor, Friend Leukemia Virus Integration 1, Transcription Factor ERGB, Ewing Sarcoma Breakpoint Region, Proto-Oncogene Fli-1, BDPLT21, EWSR2, or SIC-1.

ETS1 or protein C-ets-1 is a protein that in humans is encoded by the ETS1 gene. The protein encoded by this gene belongs to the ETS family of transcription factors. ETSI can also be referred to herein as ETS Proto-Oncogene 1 Transcription Factor, Avian Erythroblastosis Virus E26 (V-Ets) Oncogene Homolog-1, V-Ets Avian Erythroblastosis Virus E26 Oncogene Homolog 1, Protein C-Ets-1, EWSR2, P54, V-Ets Avian Erythroblastosis Virus E2 Oncogene Homolog 1, Ets Protein, C-Ets-1, or ETS-1.

In some embodiments of any of the aspects, cells are generated which or engineered to express an Ets family member selected from the group consisting of ETV2, FLI1, and ETS1.

In some embodiments, the Ets gene or protein can be ETV2 or the corresponding zebrafish etv2. In some embodiments, the Ets gene or protein can be FLI1 or the corresponding zebrafish fli1. In some embodiments, the Ets gene or protein can be ETS1 or the corresponding zebrafish ets1.

In some embodiments, the Ets gene or protein can be ETV2 and FLI1 or the corresponding zebrafish factors. In some embodiments, the Ets gene or protein can be ETV2 and ETS1 or the corresponding zebrafish factors. In some embodiments, the Ets gene or protein can be ETS1 and FLI1 or the corresponding zebrafish factors. In some embodiments, the Ets gene or protein can be ETV2, FLI1, and ETS1 or the corresponding zebrafish factors. In some embodiments, the Ets gene or protein can be ETV2, FLI1, or ETS1 or the corresponding zebrafish factors.

The amino acid sequences of the polypeptides described herein have been assigned NCBI accession numbers for different species such as human, mouse, rat, and zebrafish. In particular, the NCBI accession numbers for non-limiting examples of the amino acid sequences of human ETV2 (e.g. SEQ ID NO: 1), human FLI1 (e.g., SEQ ID NO: 2), and human ETS1 (e.g., SEQ ID NO: 3) are included herein.

(Homo sapiens ETV2, NCBI accession number
AAI40747, 342 amino acids (aa)):
SEQ ID NO: 1
MDLWNWDEASPQEVPPGNKLAGLEGAKLGFCFPDLALQGDTPTATAETCW
KGTSSSLASFPQLDWGSALLHPEVPWGAEPDSQALPWSGDWTDMACTAWD
SWSGASQTLGPAPLGPGPIPAAGSEGAAGQNCVPVAGEATSWSRAQAAGS
NTSWDCSVGPDGDTYWGSGLGGEPRTDCTISWGGPAGPDCTTSWNPGLHA
GGTTSLKRYQSSALTVCSEPSPQSDRASLARCPKTNHRGPIQLWQFLLEL
LHDGARSSCIRWTGNSREFQLCDPKEVARLWGERKRKPGMNYEKLSRGLR
YYYRRDIVRKSGGRKYTYRFGGRVPSLAYPDCAGGGRGAETQ
(Homo sapiens FLI1, NCBI accession number
AAH10115.1, 452 aa):
SEQ ID NO: 2
MDGTIKEALSVVSDDQSLFDSAYGAAAHLPKADMTASGSPDYGQPHKINP
LPPQQEWINQPVRVNVKREYDHMNGSRESPVDCSVSKCSKLVGGGESNPM
NYNSYMDEKNGPPPPNMTTNERRVIVPADPTLWTQEHVRQWLEWAIKEYS
LMEIDTSFFQNMDGKELCKMNKEDFLRATTLYNTEVLLSHLSYLRESSLL
AYNTTSHTDQSSRLSVKEDPSYDSVRRGAWGNNMNSGLNKSPPLGGAQTI
SKNTEQRPQPDPYQILGPTSSRLANPGSGQIQLWQFLLELLSDSANASCI
TWEGTNGEFKMTDPDEVARRWGERKSKPNMNYDKLSRALRYYYDKNIMTK
VHGKRYAYKFDFHGIAQALQPHPTESSMYKYPSDISYMPSYHAQQKVNFV
PPHPSSMPVTSSSFFGAASQYWTSPTGGIYPNPNVPRHPNTHVPSHLGSY
Y
(Homo sapiens ETS1, NCBI accession number
CAG47050.1, 441 aa):
SEQ ID NO: 3
MKAAVDLKPTLTIIKTEKVDLELFPSPDMECADVPLLTPSSKEMMSQALK
ATFSGFTKEQQRLGIPKDPRQWTETHVRDWVMWAVNEFSLKGVDFQKFCM
NGAALCALGKDCFLELAPDFVGDILWEHLEILQKEDVKPYQVNGVNPAYP
ESRYTSDYFISYGIEHAQCVPPSEFSEPSFITESYQTLHPISSEELLSLK
YENDYPSVILRDPLQTDTLQNDYFAIKQEVVTPDNMCMGRTSRGKLGGQD
SFESIESYDSCDRLTQSWSSQSSFNSLQRVPSYDSFDSEDYPAALPNHKP
KGTFKDYVRDRADLNKDKPVIPAAALAGYTGSGPIQLRQFLLELLTDKSC
QSFISWTGDGWEFKLSDPDEVARRWGKRKNKPKMNYEKLSRGLRYYYDKN
IIHKTAGKRYVYRFVCDLQSLLGYTPEELHAMLDVKPDADE

In some embodiments, an ETV2 amino acid or DNA sequence can be at least 90%, at least 91%, at least 92%, at least 93%, at least 94%, at least 95%, at least 96%, at least 97%, at least 98%, at least 99%, or more, identical to a native or reference sequence. In some embodiments, a FLI1 amino acid or DNA sequence can be at least 90%, at least 91%, at least 92%, at least 93%, at least 94%, at least 95%, at least 96%, at least 97%, at least 98%, at least 99%, or more, identical to a native or reference sequence. In some embodiments, an ETS amino acid or DNA sequence can be at least 90%, at least 91%, at least 92%, at least 93%, at least 94%, at least 95%, at least 96%, at least 97%, at least 98%, at least 99%, or more, identical to a native or reference sequence.

### SOX Family

SOX genes encode a family of transcription factors that bind to the minor groove in DNA, and belong to a super-family of genes characterized by a homologous sequence called the HMG-box (for high mobility group). This HMG box is a DNA binding domain that is highly conserved throughout eukaryotic species. Homologues have been identified in insects, nematodes, amphibians, reptiles, birds and a range of mammals.

Sox genes are defined as containing the HMG box of a gene involved in sex determination called SRY, which resides on the Y-chromosome (Sox stands for Sry-related HMG box). There are 20 SOX genes present in humans and mice. The family is divided into subgroups according to homology within the HMG domain and other structural motifs, as well as according to functional assays. In humans the members of the SOX groups comprise: 1) SoxA (e.g., SRY); 2) SoxB1 (e.g., SOX1, SOX2, SOX3); 3) SoxB2 (e.g., SOX14, SOX21); 4) SoxC (e.g., SOX4, SOX11, SOX12); 5) SoxD (e.g., SOX5, SOX6, SOX13); 6) SoxE (e.g., SOX8,SOX9, SOX10); 7) SoxF (e.g., SOX7, SOX17, SOX18); 8) SoxG (e.g., SOX15); 9) SoxH (e.g., SOX30).

The developmentally important Sox family has no singular function, and many members possess the ability to regulate several different aspects of development. While many Sox genes are involved in sex determination, some are also important in processes such as neuronal development. Sox proteins bind to the sequence WWCAAW and similar sequences (W=A or T). DNA motifs for the Sox family can also comprise a central ATTGT sequence (e.g., on DNA strand complementary to the first motif).

Non-limiting examples of members of the human Sox family that are relevant to endothelial niche cells comprise SOX18 and SOX7. The corresponding factors in zebrafish or Xenopus comprise sox18 and sox7.

SOX18 can also be referred to herein as SRY-Box 18, SRY (Sex Determining Region Y)-Box 18, Transcription Factor SOX-18, SRY Box 18, HLTRS, or HLTS.

SOX7 can also be referred to herein as SRY-Box 7, SRY (Sex Determining Region Y)-Box 7, Transcription Factor SOX-7, or SRY Box 7.

In some embodiments of any of the aspects, cells are generated which or engineered to express a Sox family member selected from the group consisting of SOX18 and SOX7. In some embodiments, the Sox gene or protein can be SOX18 or the corresponding zebrafish sox18 or Xenopus SOX18. In some embodiments, the Ets gene or protein can be SOX7 or the corresponding zebrafish sox7. In some embodiments, the Sox gene or protein can be SOX18 and SOX7 or the corresponding zebrafish or Xenopus factors. In some embodiments, the Sox gene or protein can be SOX18 or SOX7 or the corresponding zebrafish or Xenopus factors.

The amino acid sequences of the polypeptides described herein have been assigned NCBI accession numbers for different species such as human, mouse, rat, and zebrafish. In particular, the NCBI accession numbers for non-limiting examples of the amino acid sequences of human SOX18 (e.g. SEQ ID NO: 4), human SOX7 (e.g., SEQ ID NO: 5), and Xenopus SOX18 (e.g., SEQ ID NO: 8) are included herein.

(Homo sapiens SOX18, NCBI accession number
BAA94874.1, 384 aa):
SEQ ID NO: 4
MQRSPPGYGAQDDPPARRDCAWAPGGAAADTRGLAAGPAALAAPAAPASP
PSPQRSPPRSPEPGRYGLSPAGRGERQAADESRIRRPMNAFMVWAKDERK
RLAQQNPDLHNAVLSKMLGKAWKELNAAEKRPFVEEAERLRVQHLRDHPN
YKYRPRRKKQARKARRLEPGLLLPGLAPPQPPPEPFPAASGSARAFRELP
PLGAEFDGLGLPTPERSPLDGLEPGEAAFFPPPAAPEDCALRPFRAPYAP
TELSRDPGGCYGAPLAEALRTAPPAAPLAGLYYGTLGTPGPYPGPLSPPP
EAPPLESAEPLGPAADLWADVDLTEFDQYLNCSRTRPDAPGLPYHVALAK
LGPRAMSCPEESSLISALSDASSAVYYSACISG
(Homo sapiens SOX7, NCBI accession number,
CAC84226.1, 388 aa):
SEQ ID NO: 5
MASLLGAYPWPEGLECPALDAELSDGQSPPAVPRPPGDKGSESRIRRPMN
AFMVWAKDERKRLAVQNPDLHNAELSKMLGKSWKALTLSQKRPYVDEAER
LRLQHMQDYPNYKYRPRRKKQAKRLCKRVDPGFLLSSLSRDQNALPEKRS
GSRGALGEKEDRGEYSPGTALPSLRGCYHEGPAGGGGGGTPSSVDTYPYG
LPTPPEMSPLDVLEPEQTFFSSPCQEEHGHPRRIPHLPGHPYSPEYAPSP
LHCSHPLGSLALGQSPGVSMMSPVPGCPPSPAYYSPATYHPLHSNLQAHL
GQLSPPPEHPGFDADLQLSQVELLGDMDRNEFDQYLNTPGHPDSATGAMA
LSGHVPVSQVTPTGPTETSLISVLADATATYYNSYSVS
(Xenopus tropicalis SOX18, NCI accession number:
AAI67402.1, 362 aa):
SEQ ID NO: 8
MHRPEPSYCREEPTPCQGVNSTWVPPADTVPETSPTPSSPPAPDSPTPSP
QPGYGYSPCEEKPGDPRIRRPMNAFMVWAKDERKRLAQQNPDLHNAVLSK
MLGQSWKNLSSAEKRPFVEEAERLRVQHLQDHPNYKYRPRRKKQAKKLKR
VDPSPLLRNEGYRGQAMANLSHFRDLHPLGGSGDLESYGLPTPEMSPLDV
VEPSEPAFFPPHMREEADPGPFRTYQHGVDFGQEKTLREISLPYSSSPSH
MGGFLRTPTASAFYYNPHGGSPACTPLGQLSPPPEAPALEAMDHLGPAEL
WGDFDRNEFDQYLNMSRTQGPGYPFPMSKLGAPRTIPCEESSLISALSDA
STAMYYTPCITG

In some embodiments, a SOX7 amino acid or DNA sequence can be at least 90%, at least 91%, at least 92%, at least 93%, at least 94%, at least 95%, at least 96%, at least 97%, at least 98%, at least 99%, or more, identical to a native or reference sequence. In some embodiments, a SOX18 amino acid or DNA sequence can be at least 90%, at least 91%, at least 92%, at least 93%, at least 94%, at least 95%, at least 96%, at least 97%, at least 98%, at least 99%, or more, identical to a native or reference sequence.

### NHR Family

The Nuclear Hormone Receptor (NHR) family, also referred to as nuclear receptors, are a class of proteins found within cells that are responsible for sensing steroid and thyroid hormones and certain other molecules. In response, these receptors work with other proteins to regulate the expression of specific genes, thereby controlling the development, homeostasis, and metabolism of the organism. A unique property of nuclear receptors that differentiates them from other classes of receptors is their ability to directly interact with and control the expression of genomic DNA. As a consequence, nuclear receptors play key roles in both embryonic development and adult homeostasis. A non-limiting example of a DNA motifs for NHR family members comprises RRGGTCA, where R denotes a purine (e.g., A or G).

At least 48 nuclear receptors have been identified in humans, classified into the following subfamilies: 1) Thyroid-Hormone Receptor-like (e.g., Thyroid hormone receptor, Retinoic acid receptor, Peroxisome proliferator-activated receptor, Rev-ErbA, RAR-related orphan receptor, Liver X receptor-like, Vitamin D receptor-like, NRs with two DNA binding domains, RORA); 2) Retinoid X Receptor-like (e.g., Hepatocyte nuclear factor-4, Retinoid X receptor, Testicular receptor, TLX, PNR, COUP, EAR, RXRA, NR2F2); 3) Estrogen Receptor-like (e.g., Estrogen receptor, Estrogen related receptor, 3-Ketosteroid receptors); 4) Nerve Growth Factor IB-like (e.g., NGFIB, NURR1, NOR1); 5) Steroidogenic Factor-like (e.g., SF1, LRH1); 6) Germ Cell Nuclear Factor-like (e.g., GCNF); 7) miscellaneous nuclear receptors (e.g., DX, SHP1).

Non-limiting examples of members of the human NHR family that are relevant to endothelial niche cells comprise RXRA and NR2F2. The corresponding factors in zebrafish comprise rxraa and nr2f2.

RXRA is a nuclear receptor that belongs to the RXR transcription factor group. RXRA can also be referred to herein as Retinoid X Receptor Alpha, Nuclear Receptor Subfamily 2 Group B Member 1, Retinoic Acid Receptor RXR-Alpha, NR2B1, Retinoid X Nuclear Receptor Alpha, or Retinoid X Receptor Alpha.

The retinoid X receptor (RXR) is a type of nuclear receptor that is activated by 9-cis retinoic acid and 9-cis-13,14-dihydro-retinoic acid, which is likely to be the major endogenous mammalian RXR-selective agonist. There are three retinoic X receptors (RXR): RXR-alpha, RXR-beta, and RXR-gamma, encoded by the RXRA, RXRB, RXRG genes, respectively. RXR heterodimerizes with subfamily 1 nuclear receptors including CAR, FXR, LXR, PPAR, PXR, RAR, TR, and VDR. As with other type II nuclear receptors, the RXR heterodimer in the absence of ligand is bound to hormone response elements complexed with corepressor protein. Binding of agonist ligands to RXR results in dissociation of corepressor and recruitment of coactivator protein, which, in turn, promotes transcription of the downstream target gene into mRNA and eventually protein.

NR2F2 is a nuclear receptor that belongs to the COUP transcription factor group. NR2F2 can also be referred to herein as Nuclear Receptor Subfamily 2 Group F Member 2, Apolipoprotein A-I Regulatory Protein 1, COUP Transcription Factor II, COUP Transcription Factor 2, TFCOUP2, ARP-1, ARP1, Chicken Ovalbumin Upstream Promoter Transcription Factor 2, Chicken Ovalbumin Upstream Promoter-Transcription Factor I, Nuclear Receptor Subfamily 2 Group F Member 2, ADP-Ribosylation Factor Related Protein 1, Apolipoprotein AI Regulatory Protein 1, COUP-TF II, COUPTFII, COUP-TF2, COUPTF2, COUPTFB, CHTD4, NF-E3, or SVP40.

The chicken ovalbumin upstream promoter transcription factor (COUP-TFs) proteins are members of the nuclear receptor family of intracellular transcription factors. There are two variants of the COUP-TFs, labeled as COUP-TFI and COUP-TFII encoded by the NR2F1 and NR2F2 genes respectively. COUP-TFs play critical roles in the development of organisms.

In some embodiments of any of the aspects, cells are generated which or engineered to express an NHR family member selected from the group consisting of RXRA and NR2F2. In some embodiments, the NHR gene or protein can be RXRA or the corresponding zebrafish rxraa. In some embodiments, the NHR gene or protein can be NR2F2 or the corresponding zebrafish nr2f2. In some embodiments, the NHR gene or protein can be RXRA and NR2F2 or the corresponding zebrafish factors. In some embodiments, the NHR gene or protein can be RXRA or NR2F2 or the corresponding zebrafish factors.

The amino acid sequences of the polypeptides described herein have been assigned NCBI accession numbers for different species such as human, mouse, rat, and zebrafish. In particular, the NCBI accession numbers for non-limiting examples of the amino acid sequences of human RXRA (e.g. SEQ ID NO: 6), human NR2F2 (e.g., SEQ ID NO: 7), and zebrafish Nr2f2 (e.g., SEQ ID NO: 9) are included herein.

(Homo sapiens RXRA isoform A, NCBI accession
number NP_002948.1, 462 aa):
SEQ ID NO: 6
MDTKHFLPLDFSTQVNSSLTSPTGRGSMAAPSLHPSLGPGIGSPGQLHSP
ISTLSSPINGMGPPFSVISSPMGPHSMSVPTTPTLGFSTGSPQLSSPMNP
VSSSEDIKPPLGLNGVLVKPAHPSGNMASFTKHICAICGDRSSGKHYGVY
SCEGCKGFFKRTVRKDLTYTCRDNKDCLIDKRQRNRCQYCRYQKCLAMGM
KREAVQEERQRGKDRNENEVESTSSANEDMPVERILEAELAVEPKTETYV
EANMGLNPSSPNDPVTNICQAADKQLFTLVEWAKRIPHFSELPLDDQVIL
LRAGWNELLIASFSHRSIAVKDGILLATGLHVHRNSAHSAGVGAIFDRVL
TELVSKMRDMQMDKTELGCLRAIVLFNPDSKGLSNPAEVEALREKVYASL
EAYCKHKYPEQPGRFAKLLLRLPALRSIGLKCLEHLFFFKLIGDTPIDTF
LMEMLEAPHQMT
(Homo sapiens NR2F2 isoform A, NCBI accession
number NP_066285.1, 414 aa):
SEQ ID NO: 7
MAMVVSTWRDPQDEVPGSQGSQASQAPPVPGPPPGAPHTPQTPGQGGPAS
TPAQTAAGGQGGPGGPGSDKQQQQQHIECVVCGDKSSGKHYGQFTCEGCK
SFFKRSVRRNLSYTCRANRNCPIDQHHRNQCQYCRLKKCLKVGMRREAVQ
RGRMPPTQPTHGQFALTNGDPLNCHSYLSGYISLLLRAEPYPTSRFGSQC
MQPNNIMGIENICELAARMLFSAVEWARNIPFFPDLQITDQVALLRLTWS
ELFVLNAAQCSMPLHVAPLLAAAGLHASPMSADRVVAFMDHIRIFQEQVE
KLKALHVDSAEYSCLKAIVLFTSDACGLSDVAHVESLQEKSQCALEEYVR
SQYPNQPTRFGKLLLRLPSLRTVSSSVIEQLFFVRLVKTPIETLIRDMLL
SGSSFNWPYMAIQ
(Danio rerio Nr2f2; NCBI accession number:
AAI62484.1; 428 aa):
SEQ ID NO: 9
MAMVVWRGSQDDVAETHGTLSSQTQGGLSLPTPQPGQLGLTASQVAPPTP
QTPVQGPPNNNNNTQSTPTNQTTQSQSEKQQPQHIECVVCGDKSSGKHYG
QFTCEGCKSFFKRSVRRNLTYTCRANRNCPIDQHHRNQCQYCRLKKCLKV
GMRREVSLFTAAVQRGRMPPTQPHHGQFALTNGDPLHCHSYLSGYISLLL
RAEPYPTSRYGSQCMQPNNIMGIENICELAARMLFSAVEWARNIPFFPDL
QITDQVALLRLTWSELFVLNAAQCSMPLHVAPLLAAAGLHASPMSADRVV
AFMDHIRIFQEQVEKLKALHVDSAEYSCLKAIVLFTSDACGLSDVAHVES
LQEKSQCALEEYVRSQYPNQPTRFGKLLLRLPSLRTVSSSVIEQLFFVRL
VGKTPIETLIRDMLLSGSSFNWPYMSIQ

In some embodiments, a RXRA amino acid or DNA sequence can be at least 90%, at least 91%, at least 92%, at least 93%, at least 94%, at least 95%, at least 96%, at least 97%, at least 98%, at least 99%, or more, identical to a native or reference sequence. In some embodiments, a NR2F2 amino acid or DNA sequence can be at least 90%, at least 91%, at least 92%, at least 93%, at least 94%, at least 95%, at least 96%, at least 97%, at least 98%, at least 99%, or more, identical to a native or reference sequence.

In some embodiments, the transcription factors can be selected from the group consisting of ETV2, FLI1, ETS1, SOX18, SOX7, RXRA, and NR2F2 or the corresponding zebrafish or Xenopus factors. In some embodiments, the transcription factors can be ETV2, FLI1, ETS1, SOX18, SOX7, RXRA, or NR2F2 or the corresponding zebrafish or Xenopus factors. In some embodiments, the transcription factors can be ETV2, FLI1, ETS1, SOX18, SOX7, RXRA, and NR2F2 or the corresponding zebrafish or Xenopus factors.

In some embodiments, the transcription factors can be ETV2, SOX7, and NR2F2 or the corresponding zebrafish or Xenopus factors. In some embodiments, the transcription factors can be ETS1, SOX7, and NR2F2 or the corresponding zebrafish or Xenopus factors. In some embodiments, the transcription factors can be ETV2 alone or the corresponding zebrafish or Xenopus factor.

In some embodiments, the transcription factors can be at least 1 factor, at least 2 factors, at least 3 factors, at least 4 factors, at least 5 factors, at least 6 factors, or at least 7 factors selected from the group consisting of ETV2, FLI1, ETS1, SOX18, SOX7, RXRA, and NR2F2 or the corresponding zebrafish or Xenopus factors.

In some embodiments, transcription factors can be at ETV2 and at least 1 factor selected from the group consisting of FLI1, ETS1, SOX18, SOX7, RXRA, and NR2F2 or the corresponding zebrafish or Xenopus factors. In some embodiments, transcription factors can be at ETS1 and at least 1 factor selected from the group consisting of ETV2, FLI1, SOX18, SOX7, RXRA, and NR2F2 or the corresponding zebrafish or Xenopus factors.

### Hematopoietic System Development

The development of the haematopoietic system, including the cell populations and molecular pathways, is highly conserved between fish and mammals. HSPCs are born in the aorta-gonad-mesonephros (AGM) region and then migrate to a transient fetal niche, the fetal liver in mammals or a vascular plexus in the tail of the fish called the caudal haematopoietic tissue (CHT). HSPCs reside and expand in these developmental sites for several days before migrating to the adult niche—the bone marrow in mammals or the kidney marrow in fish.

The CHT is comprised primarily of low-flow sinusoids surrounded by mesenchymal stromal cells. HSPCs initially colonize the CHT niche by lodging within the vascular plexus and interacting directly with cxcl12a⁺ stromal cells. In a characteristic vascular remodeling step, endothelial cells (ECs) reorganize to form a supportive pocket around the HSPCs, which together with stromal cells and possibly other cell types, forms a niche for the stem cells (the endothelial cells surrounding the HSPCs can be referred to herein as endothelial niche cells). In mammals and zebrafish, specific signaling molecules, adhesion proteins and transcription factors have been implicated in mediating communication and physical interaction between stem cells and ECs in the niche. Collectively, these studies suggest that ECs within the vascular niches of haematopoietic organs express niche-specific gene programs. To date, however, a comprehensive investigation of the transcriptional circuitry that specifies the niche identity of ECs in the HSPC niche has not been undertaken. Understanding this regulation guides new strategies to improve the efficacy and availability of bone marrow transplantation therapies.

### Endothelial Niche Cells

As described herein, endothelial niche cells are endothelial cells that provide an instructive niche for the differentiation of HSPCs. Endothelial niche cells are typically found in the bone marrow. However, as described herein, exogenous expression of specific transcription factors (e.g., ETV2, FLI1, ETS1, SOX18, SOX7, RXRA, NR2F2) can cause endothelial niche cells to be found in non-bone marrow tissues, thus providing for extramedullary hematopoiesis.

In some embodiments of any of the aspects, endothelial niche cells comprise cells that express one are more genes selected from the group consisting of sele, exoc312a, snx8a, cltca, aqp7, ap1b1, lgmn, prcp, cldn11a, lyve1b, adra1d, hya12a, hya12b, ill1, i113ra2, glu1a, hexb, slc16a9a, and sepp1a. In some embodiments, the endothelial cells are human.

In some embodiments of any of the aspects, the endothelial niche cells are generated or engineered to express transcription factors, comprising at least one of the human transcription factors ETV2, FLI1, ETS1, SOX18, SOX7, RXRA, or NR2F2. In some embodiments, the transcription factor comprises at least one transcription factor from the Ets family, at least one transcription factor from the Sox family, and at least one transcription factor from the Nuclear Hormone Receptor family. In some embodiments, the transcription factors comprise ETV2, FLI1, ETS1, SOX18, SOX7, RXRA, and NR2F2.

In some embodiments of any of the aspects, the transcription factors are expressed from at least one vector. In some embodiments, the vector comprises an exogenous nucleic acid sequence or sequences encoding the one or more transcription factors. In some embodiments, the exogenous nucleic acid sequences are incorporated into the genome of the endothelial cell. As a non-limiting example, the exogenous nucleic acid sequences can be incorporated into the genome using viral vectors (e.g., AAV, lentivirus) or CRISPR technologies.

One aspect provides for a composition comprising an engineered endothelial niche cell comprising one or more exogenous nucleic acid sequences encoding one or more transcription factors, wherein the one or more transcription factors are from the Ets family, the Sox family and/or the Nuclear Hormone Family. In some embodiments of any of the aspects, the composition can comprise engineered endothelial niche cells. In some embodiments, the composition is a therapeutic agent or the composition further comprises a pharmaceutically acceptable carrier. In some embodiments, the composition further comprises a culture dish, 3D cell system, or suspension system. In some embodiments, the composition comprises a scaffold.

Another aspect provides a method for culturing HSPCs, the method comprising culturing HSPCs in the presence of a population of engineered endothelial niche cells. In some embodiments of any of the aspects, the method is performed in vitro. In some embodiments, the engineered endothelial niche cells secrete a factor (e.g., growth factors) that affects the growth and/or expansion of the HSPC cells.

In some embodiments, the HSPCs cultured in the presence of the engineered endothelial niche cells can be cultured for at least 3 days longer than HSPCs that are cultured in the absence of such engineered endothelial niche cells. In, some embodiments, the HSPCs cultured in the presence of the engineered endothelial niche cells can be cultured for at 1 day longer, at least 2 days longer, at least 3 days longer, at least 4 days longer, at least 5 days longer, at least 6 days longer, at least 7 days longer, at least 8 days longer, at least 9 days longer, at least 10 days longer, at least 11 days longer, at least 12 days longer, at least 13 days longer, or at least 14 days longer than HSPCs that are cultured in the absence of such engineered endothelial niche cells.

In some embodiments, the cells are cultured on a biologically compatible scaffold. Non-limiting examples of a biologically compatible scaffold comprise: a hydrogel, biopolymers, or another biomaterial with the ability to grow cells in vitro in preparation for transplantation. In some embodiments, the HSPCs cultured in the presence of the engineered endothelial niche cells have increased engraftment when administered to a subject compared to the engraftment of substantially similar HSPCs that were not cultured with engineered endothelial niche cells. As used herein, “engraftment” refers to the process wherein transplanted HSPCs begin to grow and produce healthy blood cells. Engraftment is a critical milestone in recovery from an HSPC transplant.

Another aspect provides a method of treating a subject, the method comprising, transplanting a composition comprising a population of engineered endothelial niche-cells into the subject. As a non-limiting example, the method can be used to treat myelofibrosis or other hematopoietic diseases where the endogenous bone marrow niche is compromised, non-limiting examples of which are disclosed herein. In some embodiments, the method can comprise transplanting a composition comprising a population of HSPCs into the subject. In some embodiments, the method can comprise transplanting a composition comprising a population of HSPCs and engineered endothelial niche-cells into the subject.

Another aspect provides a method for enhancing engraftment of HSPCs, the method comprising administering a composition comprising HSPCs and a population of engineered endothelial niche cells to a subject in need thereof. In some embodiments of any of the aspects, engraftment of the HSPCs is increased by at least 10% compared to the engraftment of substantially similar HSPCs in the absence of engineered endothelial niche cells. In some embodiments of any of the aspects, engraftment of the HSPCs is increased by at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% compared to the engraftment of substantially similar HSPCs in the absence of engineered endothelial niche cells.

Another aspect provides a co-culture comprising engineered endothelial niche cells and HSPCs. In some embodiments of any of the aspects, the endothelial cells are made by a method described herein.

Another aspect provides a method for generating an ectopic vascular niche, the method comprising: administering an engineered endothelial niche cell to a target site in a subject in need thereof. As used herein “ectopic vascular niche” refers to an atypical site for endothelial niche cells. For example, the vascular niche can be found outside of the bone marrow. The ectopic vascular niche comprising generated or engineered endothelial niche cells can be anywhere in the body. The ectopic vascular niche can be found in a location where HSPCs, generated or engineered endothelial niche cells, and/or their associated transcription factors (e.g., ETV2, FLI1, ETS1, SOX18, SOX7, RXRA, or NR2F2) have been injected.

Another aspect provides a method for extra medullary hematopoiesis, the method comprising transplanting engineered-niche endothelial cells into a subject at a location outside of the bone marrow (e.g., the forearm), thereby creating a synthetic niche. As used herein, “extra medullary hematopoiesis” refers to hematopoiesis occurring in organs outside of the bone marrow. In some embodiments of any of the aspects, the endothelial cells are made by a method described herein.

### Myelofibrosis

In some embodiments of any of the aspects, generated or engineered endothelial niche cells or their associated transcription factors (e.g., ETV2, FLI1, ETS1, SOX18, SOX7, RXRA, or NR2F2) can be used to treat myelofibrosis.

Myelofibrosis is an uncommon type of chronic leukemia. Myelofibrosis belongs to a group of diseases called myeloproliferative disorders, often of a chronic form. Chronic myeloproliferative disorders are a group of slow-growing blood cancers in which the bone marrow makes too many abnormal red blood cells, white blood cells, or platelets, which accumulate in the blood. Non-limiting examples of chronic myeloproliferative neoplasms comprise: Chronic myelogenous leukemia, Polycythemia vera, Primary myelofibrosis (also called chronic idiopathic myelofibrosis), Essential thrombocythemia, Chronic neutrophilic leukemia, and Chronic eosinophilic leukemia.

Myelofibrosis is a serious bone marrow disorder that disrupts the body's normal production of blood cells. The result is extensive scarring in bone marrow, leading to severe anemia, weakness, fatigue and often an enlarged spleen. Many subjects or patients with myelofibrosis get progressively worse, and some subjects or patients may eventually develop a more serious form of leukemia. Myelofibrosis can occur when blood stem cells (e.g., HSPCs) develop a genetic mutation. Several specific gene mutations have been identified in people with myelofibrosis. The most common is the Janus kinase 2 (JAK2) gene.

Although the cause of myelofibrosis often isn't known, certain factors are known to increase risk. Increased age can be associated with the development of myelofibrosis. Myelofibrosis can affect anyone, but it's most often diagnosed in people older than 50. Patients with another blood cell disorder are at higher risk for developing myelofibrosis. A small portion of people with myelofibrosis develop the condition as a complication of essential thrombocythemia or polycythemia vera. Exposure to certain chemicals can increase the risk for myelofibrosis. Myelofibrosis has been linked to exposure to industrial chemicals such as toluene and benzene. Exposure to radiation can increase the risk for myelofibrosis. People exposed to high levels of radiation, such as survivors of atomic bomb attacks, have an increased risk of myelofibrosis. Some people who received a radioactive contrast material called Thorotrast, used until the 1950s, have developed myelofibrosis.

Multiple complications can result from myelofibrosis. A complication of myelofibrosis can include increased pressure on blood flowing into a patient's liver. Normally, blood flow from the spleen enters the liver through a large blood vessel called the portal vein. Increased blood flow from an enlarged spleen can lead to high blood pressure in the portal vein (e.g., portal hypertension). This in turn can force excess blood into smaller veins in the stomach and esophagus, potentially causing these veins to rupture and bleed. Pain can be another complication of myelofibrosis. A severely enlarged spleen can cause abdominal pain and back pain. Myelofibrosis can lead to growths in other areas of the body. Myelofibrosis can be associated with bleeding complications. As the disease progresses, platelet count tends to drop below normal (thrombocytopenia) and platelet function becomes impaired. An insufficient number of platelets can lead to easy bleeding. Myelofibrosis can also be associated with painful bones and joints. Myelofibrosis can lead to hardening of bone marrow and inflammation of the connective tissue that is found around the bones. This may cause bone and joint pain. Myelofibrosis can also be associated with development of acute leukemia. Some patients with myelofibrosis develop acute myelogenous leukemia, a type of blood and bone marrow cancer that progresses rapidly.

Bone marrow transplantation is currently the only approved treatment for myelofibrosis. Additional treatments can only ameliorate the symptoms of myelofibrosis (e.g., anemia, enlarged spleen). Ruxolitinib, a JAK inhibitor which targets the gene mutation found in most cases of myelofibrosis, can be used to reduce symptoms of an enlarged spleen.

### Treatment Methods

As described herein, levels of functional hematopoiesis can be decreased in myelofibrosis and/or in subjects with myelofibrosis. As used herein, “functional hematopoiesis” refers to hematopoiesis that produces normal levels and proportions of blood cells (e.g., red blood cells, white blood cells, platelets). In some embodiments of any of the aspects, the level of hematopoiesis can be decreased in myelofibrosis or a myeloproliferative disorder and/or in subjects with myelofibrosis or a myeloproliferative disorder. Accordingly, in one aspect of any of the embodiments, described herein is a method of treating myelofibrosis or a myeloproliferative disorder in a subject in need thereof, the method comprising administering HSPCs, engineered endothelial cells, and/or transcription factors (e.g., ETV2, FLI1, ETS1, SOX18, SOX7, RXRA, or NR2F2) to a subject determined to have a level of functional hematopoiesis that is decreased relative to a reference. In one aspect of any of the embodiments, described herein is a method of treating myelofibrosis or a myeloproliferative disorder in a subject in need thereof, the method comprising: a) determining the level of functional hematopoiesis in a sample obtained from a subject; and b) administering HSPCs, engineered endothelial cells, and/or transcription factors (e.g., ETV2, FLI1, ETS1, SOX18, SOX7, RXRA, or NR2F2) to the subject if the level of functional hematopoiesis is decreased relative to a reference.

In some embodiments of any of the aspects, the method comprises administering HSPCs, engineered endothelial cells, and/or transcription factors (e.g., ETV2, FLI1, ETS1, SOX18, SOX7, RXRA, or NR2F2) to a subject previously determined to have a level of functional hematopoiesis that is decreased relative to a reference. In some embodiments of any of the aspects, described herein is a method of treating myelofibrosis or a myeloproliferative disorder in a subject in need thereof, the method comprising: a) first determining the level of functional hematopoiesis in a sample obtained from a subject; and b) then administering HSPCs, engineered endothelial cells, and/or transcription factors (e.g., ETV2, FLI1, ETS1, SOX18, SOX7, RXRA, or NR2F2) to the subject if the level of functional hematopoiesis is decreased relative to a reference.

In one aspect of any of the embodiments, described herein is a method of treating myelofibrosis or a myeloproliferative disorder in a subject in need thereof, the method comprising: a) determining if the subject has a decreased level of hematopoiesis; and b) administering HSPCs, engineered endothelial cells, and/or transcription factors (e.g., ETV2, FLI1, ETS1, SOX18, SOX7, RXRA, or NR2F2) to the subject if the level of functional hematopoiesis is decreased relative to a reference. In some embodiments of any of the aspects, the step of determining if the subject has a decreased level of functional hematopoiesis can comprise i) obtaining or having obtained a sample from the subject and ii) performing or having performed an assay on the sample obtained from the subject to determine/measure the level of hematopoiesis in the subject. In some embodiments of any of the aspects, the step of determining if the subject has a decreased level of functional hematopoiesis can comprise performing or having performed an assay on a sample obtained from the subject to determine/measure the level of hematopoiesis in the subject. In some embodiments of any of the aspects, the step of determining if the subject has a decreased level of functional hematopoiesis can comprise ordering or requesting an assay on a sample obtained from the subject to determine/measure the level of hematopoiesis in the subject. In some embodiments of any of the aspects, the step of determining if the subject has a decreased level of functional hematopoiesis can comprise receiving the results of an assay on a sample obtained from the subject to determine/measure the level of functional hematopoiesis in the subject. In some embodiments of any of the aspects, the step of determining if the subject has a decreased level of functional hematopoiesis can comprise receiving a report, results, or other means of identifying the subject as a subject with a decreased level of functional hematopoiesis.

In one aspect of any of the embodiments, described herein is a method of treating myelofibrosis or a myeloproliferative disorder in a subject in need thereof, the method comprising: a) determining if the subject has a decreased level of functional hematopoiesis; and b) instructing or directing that the subject be administered HSPCs, engineered endothelial niche cells, and/or transcription factors (e.g., ETV2, FLI1, ETS1, SOX18, SOX7, RXRA, or NR2F2) if the level of functional hematopoiesis is decreased relative to a reference. In some embodiments of any of the aspects, the step of determining if the subject has a decreased level of functional hematopoiesis can comprise i) obtaining or having obtained a sample from the subject and ii) performing or having performed an assay on the sample obtained from the subject to determine/measure the level of functional hematopoiesis in the subject. In some embodiments of any of the aspects, the step of determining if the subject has a decreased level of functional hematopoiesis can comprise performing or having performed an assay on a sample obtained from the subject to determine/measure the level of functional hematopoiesis in the subject. In some embodiments of any of the aspects, the step of determining if the subject has a functional level of functional hematopoiesis can comprise ordering or requesting an assay on a sample obtained from the subject to determine/measure the level of functional hematopoiesis in the subject. In some embodiments of any of the aspects, the step of instructing or directing that the subject be administered a particular treatment can comprise providing a report of the assay results. In some embodiments of any of the aspects, the step of instructing or directing that the subject be administered a particular treatment can comprise providing a report of the assay results and/or treatment recommendations in view of the assay results.

### Administration

In some embodiments, the methods described herein relate to treating a subject having or diagnosed as having myelofibrosis or a myeloproliferative disorder. Subjects having myelofibrosis or a myeloproliferative disorder can be identified by a physician using current methods of diagnosing myelofibrosis or a myeloproliferative disorder. Symptoms and/or complications of myelofibrosis or a myeloproliferative disorder which characterize these conditions and aid in diagnosis are well known in the art and include but are not limited to anemia, splenomegaly (i.e. an enlarged and painful spleen), fatigue, weak or short of breath, pain or fullness below the ribs on the left side, easy bruising, easy bleeding, excessive sweating during sleep (night sweats), fever, and/or bone pain. Tests that may aid in a diagnosis of myelofibrosis or a myeloproliferative disorder include but are not limited to a blood test (e.g., a complete blood count) or a bone marrow biopsy. Myelofibrosis or a myeloproliferative disorder can also be detected with a physical exam, imaging tests, or genetic tests. A family history of myelofibrosis or a myeloproliferative disorder, or exposure to risk factors for myelofibrosis or a myeloproliferative disorder (e.g. industrial chemicals, radiation) can also aid in determining if a subject is likely to have myelofibrosis or a myeloproliferative disorder or in making a diagnosis of myelofibrosis or a myeloproliferative disorder.

The compositions and methods described herein can be administered to a subject having or diagnosed as having myelofibrosis or a myeloproliferative disorder. In some embodiments, the methods described herein comprise administering an effective amount of compositions described herein, e.g. HSPCs, engineered endothelial niche cells, and/or transcription factors (e.g., ETV2, FLI1, ETS1, SOX18, SOX7, RXRA, or NR2F2) to a subject in order to alleviate a symptom of myelofibrosis or a myeloproliferative disorder. As used herein, “alleviating a symptom of myelofibrosis or a myeloproliferative disorder” is ameliorating any condition or symptom associated with the myelofibrosis or a myeloproliferative disorder. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic.

The term “effective amount” as used herein refers to the amount of HSPCs, engineered endothelial niche cells, and/or transcription factors (e.g., ETV2, FLI1, ETS1, SOX18, SOX7, RXRA, or NR2F2) needed to alleviate at least one or more symptom of the disease or disorder, and relates to a sufficient amount of pharmacological composition to provide the desired effect. The term “therapeutically effective amount” therefore refers to an amount of HSPCs, engineered endothelial niche cells, and/or transcription factors (e.g., ETV2, FLI1, ETS1, SOX18, SOX7, RXRA, or NR2F2) that is sufficient to provide a particular anti-myelofibrosis or anti-myeloproliferative disorder effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of HSPCs, engineered endothelial niche cells, and/or transcription factors (e.g., ETV2, FLI1, ETS1, SOX18, SOX7, RXRA, or NR2F2) which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

In some embodiments, the technology described herein relates to a pharmaceutical composition comprising HSPCs, engineered endothelial niche cells, and/or transcription factors (e.g., ETV2, FLI1, ETS1, SOX18, SOX7, RXRA, or NR2F2) as described herein, and optionally a pharmaceutically acceptable carrier. In some embodiments, the active ingredients of the pharmaceutical composition comprise HSPCs, engineered endothelial niche cells, and/or transcription factors (e.g., ETV2, FLI1, ETS1, SOX18, SOX7, RXRA, or NR2F2) as described herein. In some embodiments, the active ingredients of the pharmaceutical composition consist essentially of HSPCs, engineered endothelial niche cells, and/or transcription factors (e.g., ETV2, FLI1, ETS1, SOX18, SOX7, RXRA, or NR2F2) as described herein. In some embodiments, the active ingredients of the pharmaceutical composition consist of HSPCs, engineered endothelial niche cells, and/or transcription factors (e.g., ETV2, FLI1, ETS1, SOX18, SOX7, RXRA, or NR2F2) as described herein.

Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g. HSPCs, engineered endothelial niche cells, and/or transcription factors (e.g., ETV2, FLI1, ETS1, SOX18, SOX7, RXRA, or NR2F2) as described herein.

In some embodiments, the pharmaceutical composition comprising HSPCs, engineered endothelial niche cells, and/or transcription factors (e.g., ETV2, FLI1, ETS1, SOX18, SOX7, RXRA, or NR2F2) as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of HSPCs, engineered endothelial niche cells, and/or transcription factors (e.g., ETV2, FLI1, ETS1, SOX18, SOX7, RXRA, or NR2F2) as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

In some embodiments of any of the aspects, the HSPCs, engineered endothelial niche cells, and/or transcription factors (e.g., ETV2, FLI1, ETS1, SOX18, SOX7, RXRA, or NR2F2) described herein is administered as a monotherapy, e.g., another treatment for the myelofibrosis or a myeloproliferative disorder is not administered to the subject.

In some embodiments of any of the aspects, the methods described herein can further comprise administering a second agent and/or treatment to the subject, e.g. as part of a combinatorial therapy. Non-limiting examples of a second agent and/or treatment can include radiation therapy, surgery, gemcitabine, cisplastin, paclitaxel, carboplatin, bortezomib, AMG479, vorinostat, rituximab, temozolomide, rapamycin, ABT-737, PI-103; alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaIl (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE.®. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb.RTM.); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

In addition, the methods of treatment can further include the use of radiation or radiation therapy. Further, the methods of treatment can further include the use of surgical treatments.

The methods described herein can further comprise administering a second agent and/or treatment to the subject, e.g. as part of a combinatorial therapy. By way of non-limiting example, if a subject is to be treated for pain or inflammation according to the methods described herein, the subject can also be administered a second agent and/or treatment known to be beneficial for subjects suffering from pain or inflammation. In some embodiments, the second agent is an anti-inflammation agent. Examples of such agents and/or treatments include, but are not limited to, non-steroidal anti-inflammatory drugs (NSAIDs—such as aspirin, ibuprofen, or naproxen); corticosteroids, including glucocorticoids (e.g. cortisol, prednisone, prednisolone, methylprednisolone, dexamethasone, betamethasone, triamcinolone, and beclometasone); methotrexate; sulfasalazine; leflunomide; anti-TNF medications; cyclophosphamide; pro-resolving drugs; mycophenolate; or opiates (e.g. endorphins, enkephalins, and dynorphin), steroids, analgesics, barbiturates, oxycodone, morphine, lidocaine, and the like.

In certain embodiments, an effective dose of a composition comprising HSPCs, engineered endothelial niche cells, and/or transcription factors (e.g., ETV2, FLI1, ETS1, SOX18, SOX7, RXRA, or NR2F2) as described herein can be administered to a patient once. In certain embodiments, an effective dose of a composition comprising HSPCs, engineered endothelial niche cells, and/or transcription factors (e.g., ETV2, FLI1, ETS1, SOX18, SOX7, RXRA, or NR2F2) can be administered to a patient repeatedly. For systemic administration, subjects can be administered a therapeutic amount of a composition comprising HSPCs, engineered endothelial niche cells, and/or transcription factors (e.g., ETV2, FLI1, ETS1, SOX18, SOX7, RXRA, or NR2F2), such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. HSPCs, engineered endothelial niche cells, and/or transcription factors (e.g., ETV2, FLI1, ETS1, SOX18, SOX7, RXRA, or NR2F2) by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to HSPCs, engineered endothelial niche cells, and/or transcription factors (e.g., ETV2, FLI1, ETS1, SOX18, SOX7, RXRA, or NR2F2). The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition comprising HSPCs, engineered endothelial niche cells, and/or transcription factors (e.g., ETV2, FLI1, ETS1, SOX18, SOX7, RXRA, or NR2F2) can be administered over a period of time, such as over a 5 minute, 10 minute, 15 minute, 20 minute, or 25 minute period.

The dosage ranges for the administration of HSPCs, engineered endothelial niche cells, and/or transcription factors (e.g., ETV2, FLI1, ETS1, SOX18, SOX7, RXRA, or NR2F2), according to the methods described herein depend upon, for example, its form, its potency, and the extent to which symptoms, markers, or indicators of a condition described herein are desired to be reduced, for example the extent to which, for example, myelofibrosis or a myeloproliferative disorder is desired to be reduced functional hematopoiesis is desired to be induced. The dosage should not be so large as to cause adverse side effects, such as excessive hematopoiesis or excessive extramedullary hematopoiesis. Generally, the dosage will vary with the age, condition, and sex of the patient and can be determined by one of skill in the art. The dosage can also be adjusted by the individual physician in the event of any complication.

The efficacy of HSPCs, engineered endothelial niche cells, and/or transcription factors (e.g., ETV2, FLI1, ETS1, SOX18, SOX7, RXRA, or NR2F2) in, e.g. the treatment of a condition described herein, or to induce a response as described herein (e.g. functional hematopoiesis) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. blood cell counts. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example treatment of myelofibrosis or a myeloproliferative disorder. When using an experimental animal model, efficacy of treatment is evidenced when a statistically significant change in a marker is observed.

### Kits

One aspect described herein provides a kit for culturing HSPCs, the kit comprising: a population of engineered endothelial niche cells, reagents and instructions for use thereof. Another aspect provides for a kit for generating engineered endothelial niche cells comprising: a vector(s) comprising one or more exogenous nucleic acid sequences encoding one or more transcription factors of the Ets family, the Sox family or the nuclear hormone family and instructions for use thereof. Described herein are kit components that can be included in one or more of the kits described herein.

In some embodiments, the kit comprises an effective amount of reagents for culturing HSPCs and/or endothelial niche cells. As will be appreciated by one of skill in the art, reagents can be supplied in a lyophilized form or a concentrated form that can diluted prior to use with cultured cells. Preferred formulations include those that are non-toxic to the cells and/or does not affect growth rate or viability etc. reagents can be supplied in aliquots or in unit doses.

In some embodiments the kit further comprises a vector comprising a nucleic acid encoding a gene to one or more transcription factors of the Ets family, the Sox family or the nuclear hormone family under the control of a promoter.

In some embodiments, the components described herein can be provided singularly or in any combination as a kit. The kit includes the components described herein, e.g., a composition comprising HSPCs, engineered endothelial niche cells, and/or transcription factors (e.g., ETV2, FLI1, ETS1, SOX18, SOX7, RXRA, or NR2F2), a composition(s) that includes a vector comprising e.g., a gene to one or more transcription factors of the Ets family, the Sox family or the nuclear hormone family under the control of a promoter as described throughout the specification. In addition, the kit optionally comprises informational material. The kit can also contain culture dishes and/or a substrate for coating culture dishes, such as laminin, fibronectin, Poly-L-Lysine, or methylcellulose.

In some embodiments, the compositions in the kit can be provided in a watertight or gas tight container which in some embodiments is substantially free of other components of the kit. For example, a HSPCs, engineered endothelial niche cells, and/or transcription factors (e.g., ETV2, FLI1, ETS1, SOX18, SOX7, RXRA, or NR2F2) composition can be supplied in more than one container, e.g., it can be supplied in a container having sufficient reagent for a predetermined number of experiments, e.g., 1, 2, 3 or greater. One or more components as described herein can be provided in any form, e.g., liquid, dried or lyophilized form. It is preferred that the components described herein are substantially pure and/or sterile. When the components described herein are provided in a liquid solution, the liquid solution preferably is an aqueous solution, with a sterile aqueous solution being preferred.

The informational material can be descriptive, instructional, marketing or other material that relates to the methods described herein. The informational material of the kits is not limited in its form. In one embodiment, the informational material can include information about production of endothelial niche cells and/or HSPCs, concentration, date of expiration, batch or production site information, and so forth. In one embodiment, the informational material relates to methods for using or administering the components of the kit.

The kit can include a component for the detection of a marker for HSPC differentiation and/or endothelial niche cell differentiation. In addition, the kit can include one or more antibodies that bind a cell marker, or primers for an RT-PCR or PCR reaction, e.g., a semi-quantitative or quantitative RT-PCR or PCR reaction. Such components can be used to assess the activation of maturation markers or the loss of immature cell markers of endothelial niche cells and/or HSPCs. If the detection reagent is an antibody, it can be supplied in dry preparation, e.g., lyophilized, or in a solution. The antibody or other detection reagent can be linked to a label, e.g., a radiological, fluorescent (e.g., GFP) or colorimetric label for use in detection. If the detection reagent is a primer, it can be supplied in dry preparation, e.g., lyophilized, or in a solution.

The kit will typically be provided with its various elements included in one package, e.g., a fiber-based, e.g., a cardboard, or polymeric, e.g., a Styrofoam box. The enclosure can be configured so as to maintain a temperature differential between the interior and the exterior, e.g., it can provide insulating properties to keep the reagents at a preselected temperature for a preselected time.

### Vectors

In some embodiments, one or more of the factors described herein is expressed in a recombinant expression vector or plasmid. As used herein, the term “vector” refers to a polynucleotide sequence suitable for transferring transgenes into a host cell. The term “vector” includes plasmids, mini-chromosomes, phage, naked DNA and the like. See, for example, U.S. Pat. Nos. 4,980,285; 5,631,150; 5,707,828; 5,759,828; 5,888,783 and, 5,919,670, and, Sambrook et al, Molecular Cloning: A Laboratory Manual, 2nd Ed., Cold Spring Harbor Press (1989). One type of vector is a “plasmid,” which refers to a circular double stranded DNA loop into which additional DNA segments are ligated. Another type of vector is a viral vector, wherein additional DNA segments are ligated into the viral genome. Certain vectors are capable of autonomous replication in a host cell into which they are introduced (e.g., bacterial vectors having a bacterial origin of replication and episomal mammalian vectors). Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” is used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

A cloning vector is one which is able to replicate autonomously or integrated in the genome in a host cell, and which is further characterized by one or more endonuclease restriction sites at which the vector may be cut in a determinable fashion and into which a desired DNA sequence can be ligated such that the new recombinant vector retains its ability to replicate in the host cell. In the case of plasmids, replication of the desired sequence can occur many times as the plasmid increases in copy number within the host cell such as a host bacterium or just a single time per host before the host reproduces by mitosis. In the case of phage, replication can occur actively during a lytic phase or passively during a lysogenic phase.

An expression vector is one into which a desired DNA sequence can be inserted by restriction and ligation such that it is operably joined to regulatory sequences and can be expressed as an RNA transcript. Vectors can further contain one or more marker sequences suitable for use in the identification of cells which have or have not been transformed or transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). In certain embodiments, the vectors used herein are capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

When the nucleic acid molecule that encodes any of the factors/polypeptides described herein is expressed in a cell, a variety of transcription control sequences (e.g., promoter/enhancer sequences) can be used to direct its expression. The promoter can be a native promoter, i.e., the promoter of the gene in its endogenous context, which provides normal regulation of expression of the gene. In some embodiments the promoter can be constitutive, i.e., the promoter is unregulated allowing for continual transcription of its associated gene. A variety of conditional promoters also can be used, such as promoters controlled by the presence or absence of a molecule.

The precise nature of the regulatory sequences needed for gene expression can vary between species or cell types, but in general can include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences can also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA). That heterologous DNA (RNA) is placed under operable control of transcriptional elements to permit the expression of the heterologous DNA in the host cell.

In some embodiments, the vector is pME Gateway vector (Invitrogen™). In some embodiments, the vector is p5E Gateway™ vector. In some other embodiments, the vector is pGEX2TK™ vector. In some other embodiments, the vector is TOPO-TA™ vector.

Without limitations, the genes described herein can be included in one vector or separate vectors. For example, at least one gene from the Ets family (e.g., ETV2, FLI1, ETS1) and at least one gene from the SOX family (e.g., SOX18, SOX7) and at least one gene from the NHR family (e.g., RXRA, NR2F2) can be included in the same vector.

In some embodiments, at least one gene from the Ets family (e.g., ETV2, FLI1, ETS1) and at least one gene from the SOX family (e.g., SOX18, SOX7) can be included in a first vector, and at least one gene from the NHR family (e.g., RXRA, NR2F2) can be included in a second vector.

In some embodiments, at least one gene from the NHR family (e.g., RXRA, NR2F2) and at least one gene from the SOX family (e.g., SOX18, SOX7) can be included in a first vector, and at least one gene from the Ets family (e.g., ETV2, FLI1, ETS1) can be included in a second vector.

In some embodiments, at least one gene from the Ets family (e.g., ETV2, FLI1, ETS1) and at least one gene from the NHR family (e.g., RXRA, NR2F2) can be included in a first vector, and at least one gene from the SOX family (e.g., SOX18, SOX7) can be included in a second vector.

In some embodiments, at least one gene from the Ets family (e.g., ETV2, FLI1, ETS1) can be included in a first vector, at least one gene from the SOX family (e.g., SOX18, SOX7) can be included in a second vector, and at least one gene from the NHR family (e.g., RXRA, NR2F2) can be included in a third vector.

In some embodiments, the promoter operably linked to the gene(s) can be zebrafish ubi promoter.

In some embodiments, one or more of the recombinantly expressed gene can be integrated into the genome of the cell.

A nucleic acid molecule that encodes the enzyme of the claimed invention can be introduced into a cell or cells using methods and techniques that are standard in the art. For example, nucleic acid molecules can be introduced by standard protocols such as transformation including chemical transformation and electroporation, transduction, particle bombardment, etc. Expressing the nucleic acid molecule encoding the enzymes of the claimed invention also may be accomplished by integrating the nucleic acid molecule into the genome.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of myelofibrosis or a myeloproliferative disorder. A subject can be male or female.

A subject can be one who has been previously diagnosed with or identified as suffering from or having a condition in need of treatment (e.g. myelofibrosis or a myeloproliferative disorder) or one or more complications related to such a condition, and optionally, have already undergone treatment for myelofibrosis or a myeloproliferative disorder or the one or more complications related to myelofibrosis or a myeloproliferative disorder. Alternatively, a subject can also be one who has not been previously diagnosed as having myelofibrosis or a myeloproliferative disorder or one or more complications related to myelofibrosis or a myeloproliferative disorder. For example, a subject can be one who exhibits one or more risk factors for myelofibrosis or a myeloproliferative disorder or one or more complications related to myelofibrosis or a myeloproliferative disorder or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. transcription factor activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser(S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

In some embodiments, the expression of a biomarker(s), target(s), or gene/polypeptide described herein is/are tissue-specific. In some embodiments, the expression of a biomarker(s), target(s), or gene/polypeptide described herein is/are global. In some embodiments, the expression of a biomarker(s), target(s), or gene/polypeptide described herein is systemic.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

“Marker” in the context of the present invention refers to an expression product, e.g., nucleic acid or polypeptide which is differentially present in a sample taken from subjects having myelofibrosis or a myeloproliferative disorder, as compared to a comparable sample taken from control subjects (e.g., a healthy subject). The term “biomarker” is used interchangeably with the term “marker.”

In some embodiments, the methods described herein relate to measuring, detecting, or determining the level of at least one marker. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments of any of the aspects, the HSPCs, engineered endothelial niche cells, and/or transcription factors (e.g., ETV2, FLI1, ETS1, SOX18, SOX7, RXRA, or NR2F2) described herein is exogenous. In some embodiments of any of the aspects, the HSPCs, engineered endothelial niche cells, and/or transcription factors (e.g., ETV2, FLI1, ETS1, SOX18, SOX7, RXRA, or NR2F2) described herein is ectopic. In some embodiments of any of the aspects, the HSPCs, engineered endothelial niche cells, and/or transcription factors (e.g., ETV2, FLI1, ETS1, SOX18, SOX7, RXRA, or NR2F2) described herein is not endogenous.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

In some embodiments, a nucleic acid encoding a polypeptide as described herein (e.g. a ETV2, FLI1, ETS1, SOX18, SOX7, RXRA, or NR2F2 polypeptide) is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments of any of the aspects, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments of any of the aspects, the vector or nucleic acid described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments of any of the aspects, the expression system is an organism other than the source of the native/wild-type sequence (or a cell obtained from such organism). In some embodiments of any of the aspects, the vector and/or nucleic acid sequence described herein is codon-optimized for expression in a mammal or mammalian cell, e.g., a mouse, a murine cell, or a human cell. In some embodiments of any of the aspects, the vector and/or nucleic acid sequence described herein is codon-optimized for expression in a human cell. In some embodiments of any of the aspects, the vector and/or nucleic acid sequence described herein is codon-optimized for expression in a yeast or yeast cell. In some embodiments of any of the aspects, the vector and/or nucleic acid sequence described herein is codon-optimized for expression in a bacterial cell. In some embodiments of any of the aspects, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. myelofibrosis or a myeloproliferative disorder. The term “treating” includes reducing or alleviating at least one adverse effect or symptom of a condition, disease or disorder associated with myelofibrosis or a myeloproliferative disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A method to generate/engineer endothelial niche cells, comprising expressing one or more transcription factors in an endothelial cell, wherein the one or more transcription factors are from the Ets family, the Sox family, and/or the Nuclear Hormone Receptor family.
2. The method of any one of the above paragraphs, wherein the endothelial niche cells express one are more genes comprising; sele, exoc312a, snx8a, cltca, aqp7, ap1b1, lgmn, prcp, cldn11a, lyve1b, adra1d, hya12a, hya12b, tll1, i113ra2, glu1a, hexb, slc16a9a, or sepp1a.
3. The method of any one of the above paragraphs, wherein the endothelial cells are human.
4. The method of any one of the above paragraphs, wherein the transcription factors comprises at least one of the human transcription factors ETV2, FLI1, ETS1, SOX18, SOX7, RXRA, or NR2F2.
5. The method of any one of the above paragraphs, wherein the transcription factor includes at least one transcription factor from the Ets family, at least one transcription factor from the Sox family, and at least one transcription factor from the Nuclear Hormone Receptor family.
6. The method of any one of the above paragraphs, wherein the transcription factors include ETV2, FLI1, ETS1, SOX18, SOX7, RXRA, and NR2F2.
7. The method of any one of the above paragraphs, wherein the transcription factors are expressed from at least one vector.
8. The method of any one of the above paragraphs, wherein the vector comprises an exogenous nucleic acid sequence(s) encoding the one or more transcription factors.
9. The method of any one of the above paragraphs, wherein the exogenous nucleic acid sequences are incorporated into the genome of the endothelial cell.
10. An engineered endothelial niche cell comprising one or more exogenous nucleic acid sequences encoding one or more transcription factors, wherein the one or more transcription factors are from the Ets family, the Sox family and/or the Nuclear Hormone Family.
11. A composition comprising the engineered endothelial niche cells of paragraph 10.
12. The composition of any one of the above paragraphs, wherein the composition is a therapeutic agent or the composition further comprises a pharmaceutically acceptable carrier.
13. The composition of any one of the above paragraphs, wherein the composition further comprises a culture dish, 3D cell system, or suspension system.
14. The composition of any one of the above paragraphs, wherein the composition comprises a scaffold.
15. A method for culturing HSPCs, the method comprising culturing HSPCs in the presence of a population of engineered endothelial niche cells.
16. The method of any one of the above paragraphs, wherein the method is performed in vitro.
17. The method of any one of the above paragraphs, wherein the engineered endothelial niche cells secrete a factor that affects the growth and/or expansion of the HSPC cells.
18. The method of any one of the above paragraphs, wherein the HSPCs cultured in the presence of the engineered endothelial niche cells can be cultured for at least 3 (e.g., at least 4, at least 5, at least 6, at least 7) days longer than HSPCs that are cultured in the absence of such engineered endothelial niche cells.
19. The method of any one of the above paragraphs, wherein the cells are cultured on a biologically compatible scaffold.
20. The method of any one of the above paragraphs, wherein the HSPCs cultured in the presence of the engineered endothelial niche cells have increased engraftment when administered to a subject compared to the engraftment of substantially similar HSPCs that were not cultured with engineered endothelial niche cells.
21. A method of treating a subject, the method comprising, transplanting a composition comprising HSPCs and a population of engineered endothelial niche-cells into the subject.
22. A method for enhancing engraftment of HSPCs, the method comprising administering a composition comprising HSPCs and a population of engineered endothelial niche cells to a subject in need thereof.
23. The method of any one of the above paragraphs, wherein engraftment of the HSPCs is increased by at least 10% compared to the engraftment of substantially similar HSPCs in the absence of engineered endothelial niche cells.
24. A co-culture comprising engineered endothelial niche cells and HSPCs.
25. The co-culture of paragraph 24 wherein the endothelial cells are made by the method of any one of the above paragraphs.
26. A kit for culturing HSPCs, the kit comprising: a population of engineered endothelial niche cells, reagents and instructions for use thereof.
27. A kit for generating engineered endothelial niche cells comprising: a vector(s) comprising one or more exogenous nucleic acid sequences encoding one or more transcription factors of the Ets family, the Sox family or the nuclear hormone family and instructions for use thereof.
28. A method for generating an ectopic vascular niche, the method comprising: administering an engineered endothelial niche cell to a target site in a subject in need thereof.
29. A method for extra medullary hematopoiesis, the method comprising transplanting engineered-niche endothelial cells into a subject at a location outside of the bone marrow (e.g., the forearm), thereby creating a synthetic niche.
30. The method of any one of the above paragraphs, wherein the endothelial cells are made by any of the methods in the above paragraphs.
31. A vector comprising one or more exogenous nucleic acid sequences encoding one or more transcription factors of the Ets family, the Sox family or the nuclear hormone family operably linked to a promoter.

### EXAMPLES

### Example 1

### Transcription Factor Induction of Vascular Blood Stem Cell Niches In Vivo.

The haematopoietic niche is a supportive in vivo microenvironment comprised of distinct cell types, including specialized vascular endothelial cells that directly interact with haematopoietic stem and progenitor cells (HSPCs) to facilitate stem cell function. The molecular factors that specify niche endothelial cells and their pro-haematopoietic activity remain largely unknown. Using multi-dimensional gene expression analyses and a chromatin accessibility assay, defined herein is a conserved gene expression signature and cis-regulatory landscape unique to sinusoidal endothelial cells in the HSPC niche. Using enhancer mutagenesis and transcription factor overexpression, a transcriptional code was elucidated involving members of the Ets, Sox and Nuclear Hormone Receptor families that is sufficient to induce ectopic niche endothelial cells that recruit HSPCs and support their homeostasis in vivo. Together, these studies have important implications for generating more efficient synthetic vascular niches for blood stem cells or for modulating the niche in a therapeutic context.

### Results

### An Endothelial Gene Expression Signature Unique to the Fetal HSPC Niche.

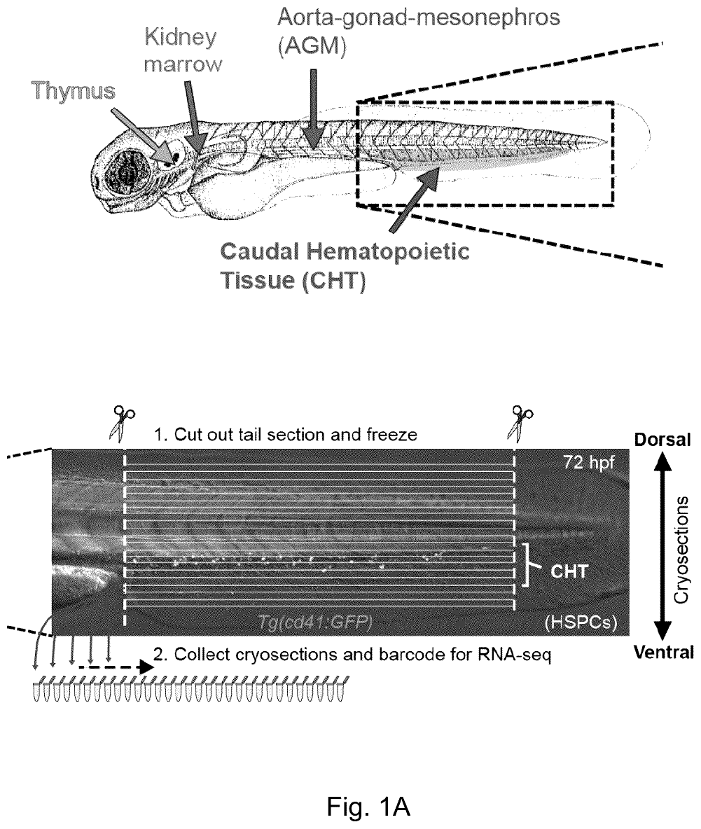

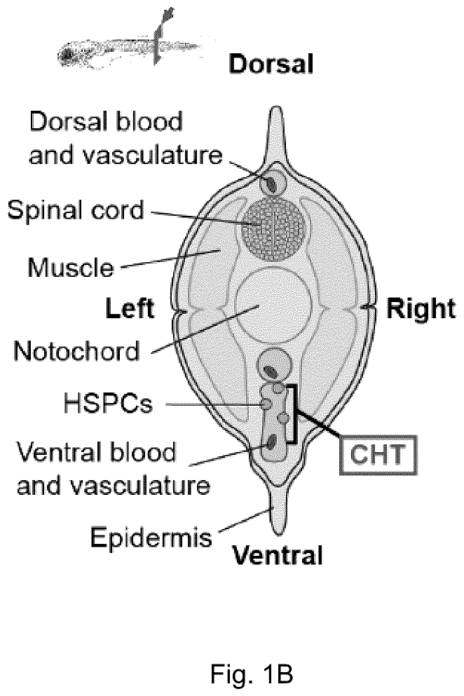

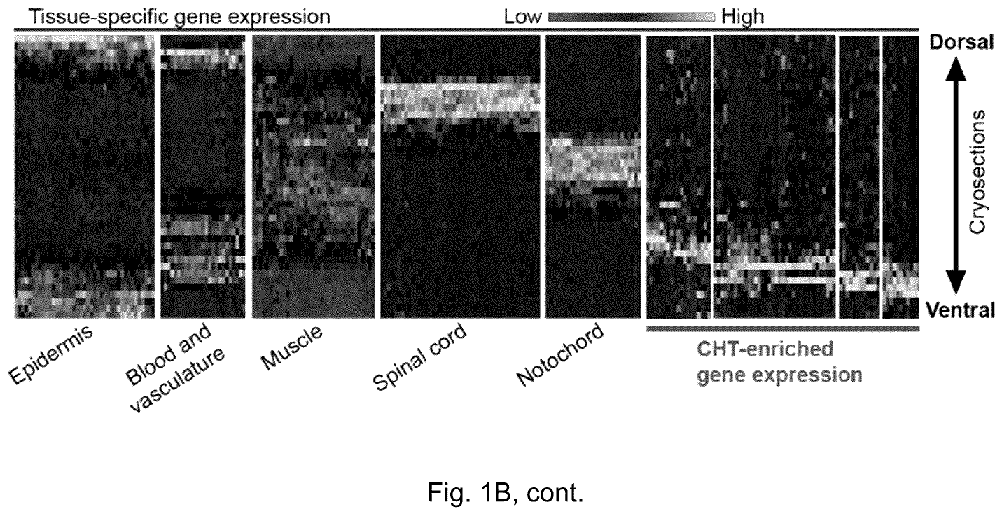

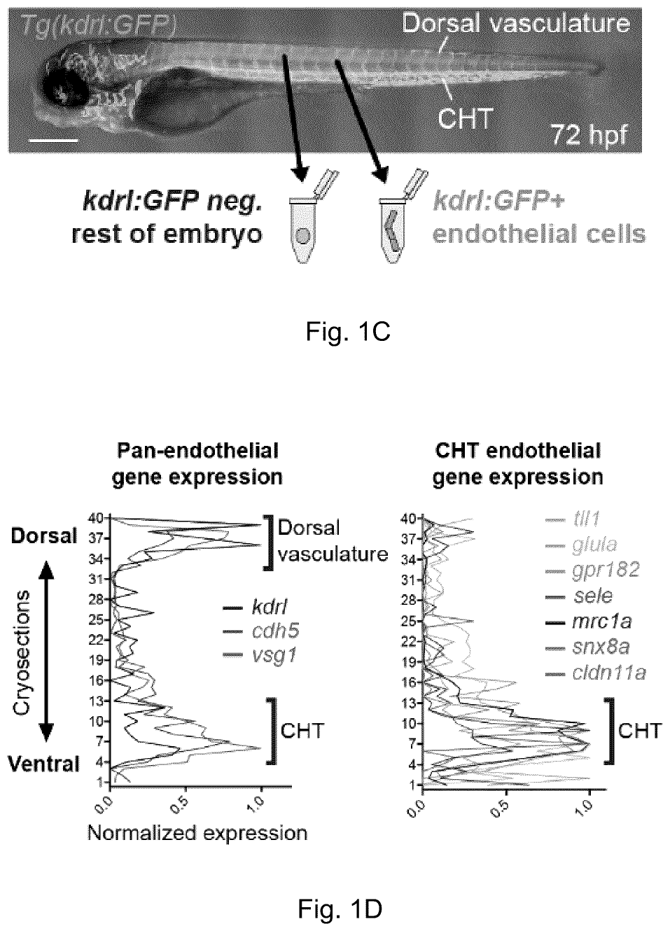

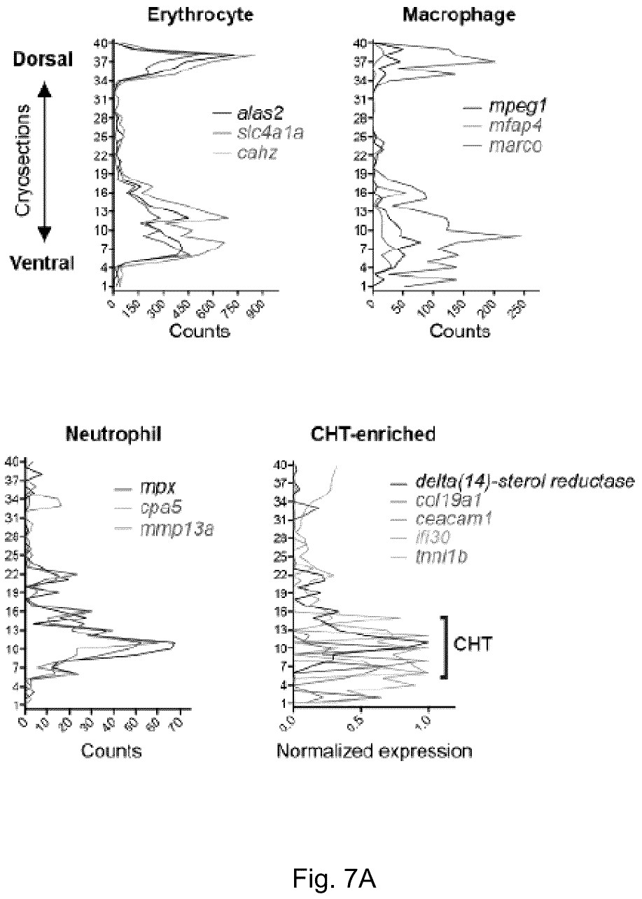

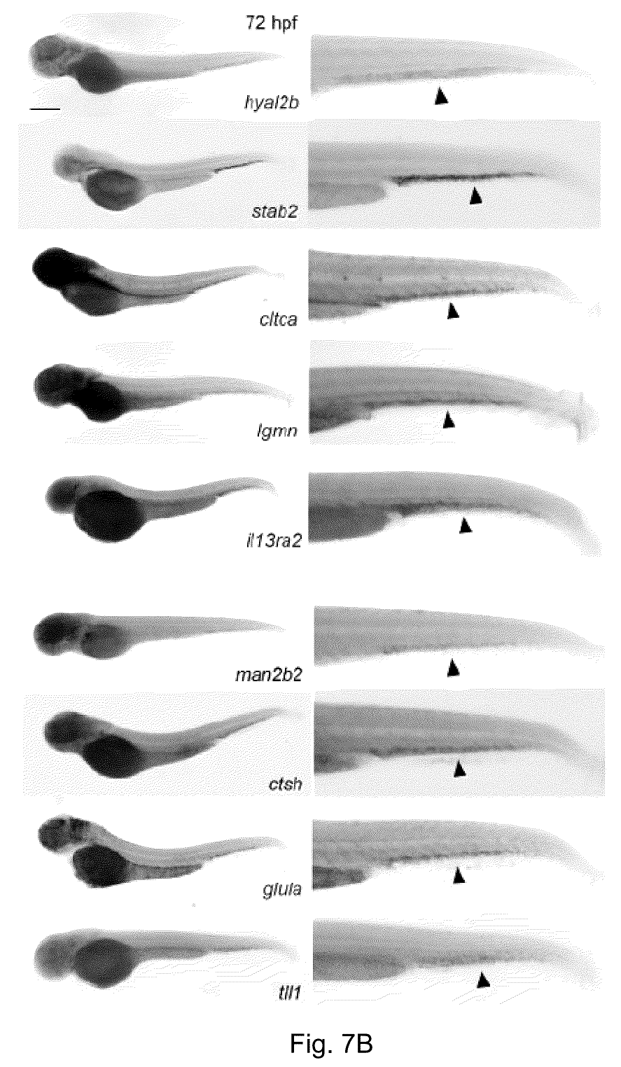

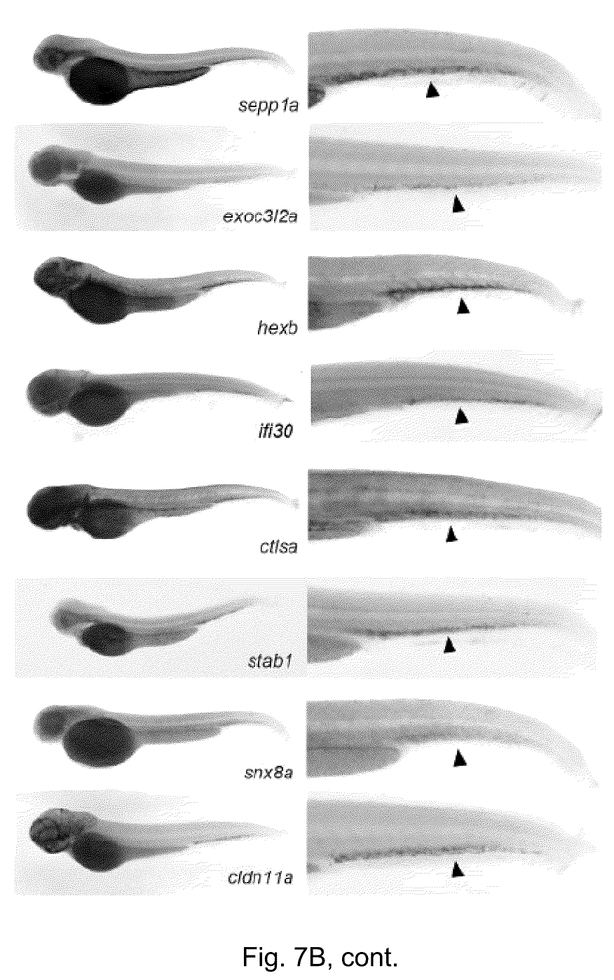

ECs from different organs express distinct genes, but whether this is regulated by organ-specific transcriptional programs remains poorly understood. To investigate the regulation of gene expression in the CHT niche, RNA tomography (tomo-seq) was performed on the zebrafish tail at 72 hours post fertilization (hpf; see e.g., FIG. 1A). This tomo-seq analysis revealed clusters of gene expression corresponding to specific tissues along the dorsal-ventral axis of the tail, including spinal cord, notochord, muscle and epidermis, as well as specific blood and immune cell populations (see e.g., FIG. 1B, FIG. 7A, FIG. 7B). In total, 144 genes displayed enriched expression within the few cryosections spanning the CHT (see e.g., Table 3). Using whole mount in situ hybridization (WISH), CHT expression of 35 of these genes was confirmed (see e.g., Table 3; images available on the world wide web at zfin.org). To determine whether any of the 144 genes were expressed by ECs, the pan-endothelial transgene kdrl:GFP and fluorescence activated cell sorting (FACS) was used to isolate ECs for bulk and single cell RNA-seq (see e.g., FIG. 1C). In addition, these genes were cross-referenced with macrophage and neutrophil RNA-seq datasets (see e.g., Theodore et al. Distinct Roles for Matrix Metalloproteinases 2 and 9 in Embryonic Hematopoietic Stem Cell Emergence, Migration, and Niche Colonization. Stem cell reports 8, 1226-1241, 2017). Twenty-nine genes were identified that were selectively enriched in CHT ECs (see e.g., Table 1). In contrast to pan-endothelial genes, the tomo-seq expression traces for these CHT endothelial genes lacked the strong peak corresponding to expression in dorsal vasculature (see e.g., FIG. 1D). For 25 out of 29 genes the CHT EC-enriched expression was confirmed by WISH (see e.g., FIG. 1E, FIG. 7A, FIG. 7B, and Table 1).

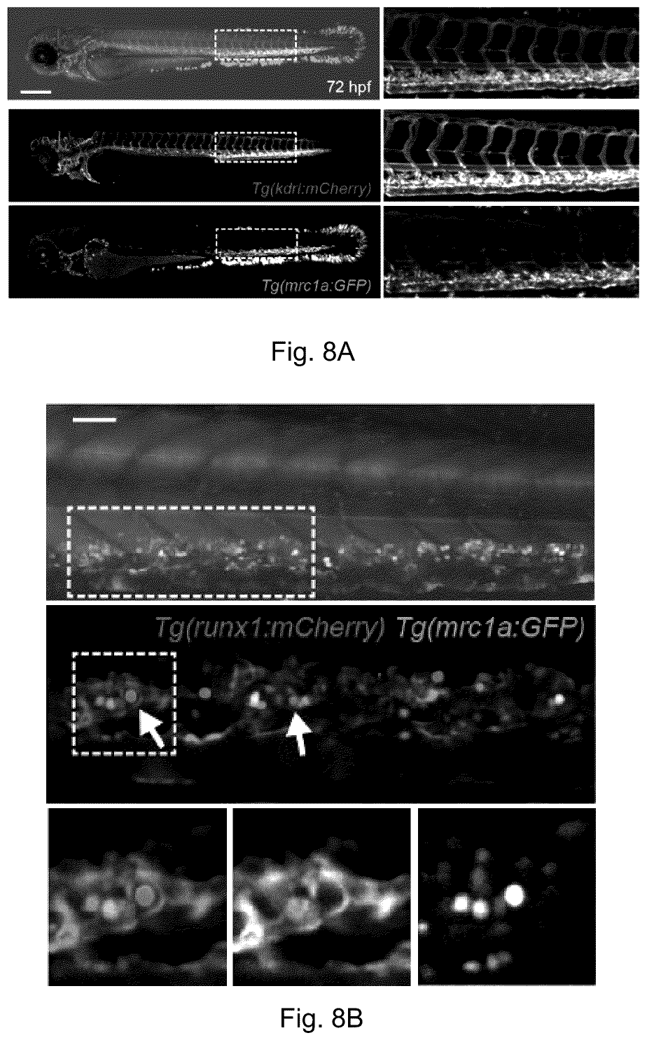

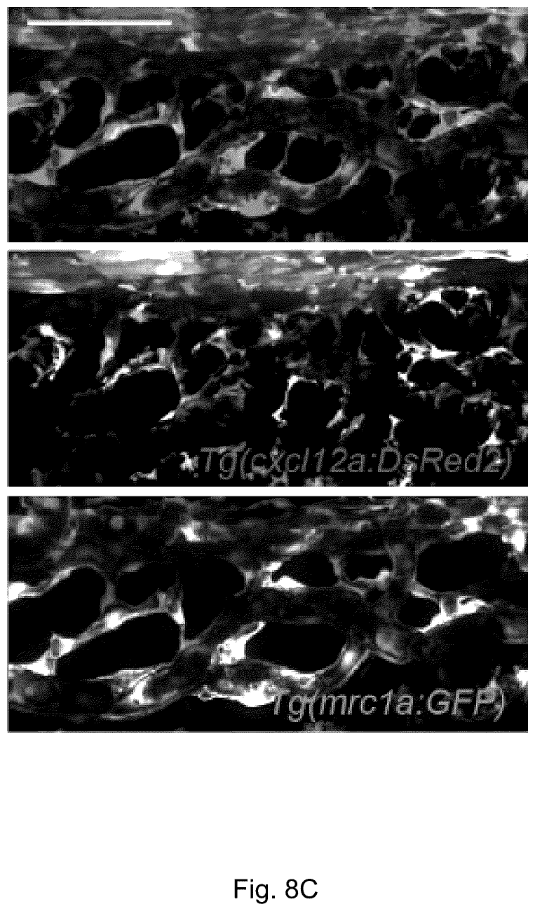

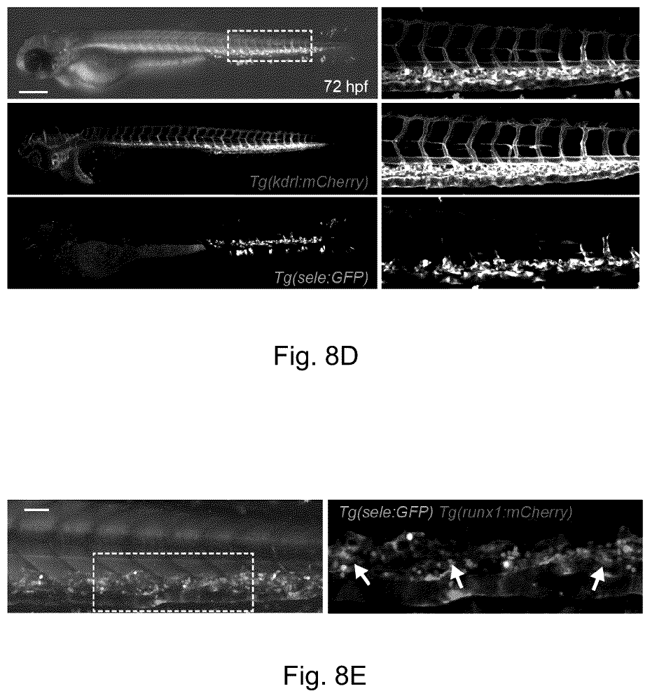

To selectively isolate CHT ECs, transgenic lines were designed to label these cells. 1.3 or 5.3 kb upstream regulatory sequences were cloned for two CHT endothelial genes, mrc1a and sele to generate GFP reporter transgenes that were then crossed to the pan-endothelial marker kdrl:mCherry. For both the mrc1a:GFP and sele:GFP transgenes, the highest levels of expression were observed in the venous sinusoids of the CHT, while low levels of GFP expression were detected in the posterior cardinal vein above the yolk extension and in a small number of vessels in the head (see e.g., FIG. 8A-FIG. 8E). Although GFP expression was also observed in mesenchymal cells in the outer tail fin, these cells are a frequent site of ectopic transgene expression and do not represent the endogenous expression of these genes. Notably, robust GFP expression was observed in sinusoidal ECs that directly interacted with HSPCs, confirming that the mrc1a:GFP and sele:GFP transgenes labeled ECs in the HSPC niche (see e.g., FIG. 8A-FIG. 8E). These GFP⁺ ECs closely associated with cxcl12a:DsRed2⁺ stromal cells and were observed to form pockets around HSPCs—a cellular behavior characteristic of ECs in the CHT niche (see e.g., FIG. 8A-FIG. 8E).

### Endothelial Niche-Specific Cis-Regulatory Elements.

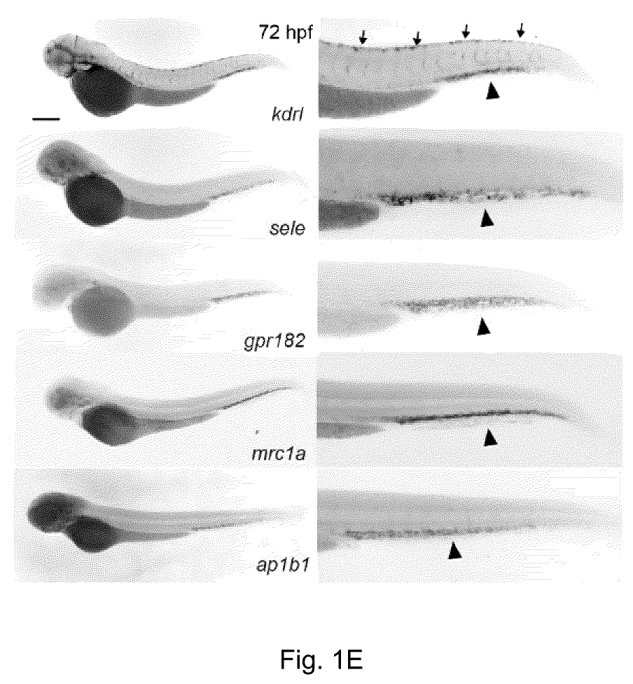

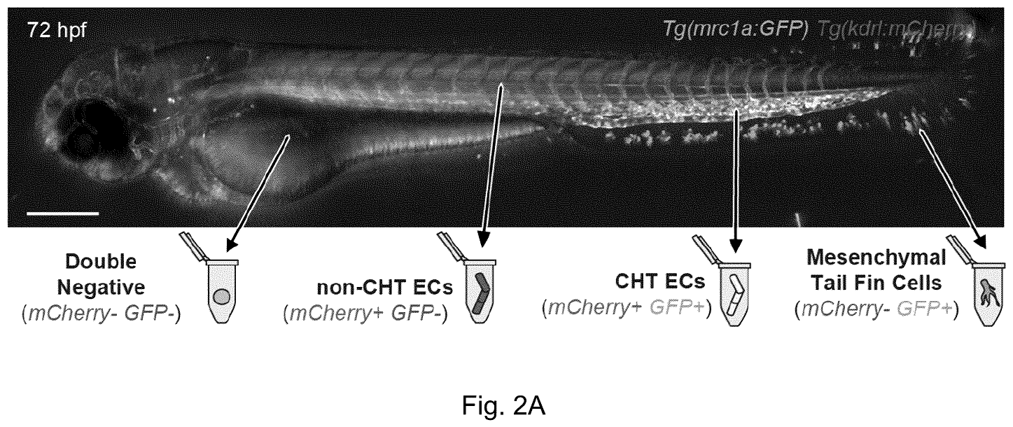

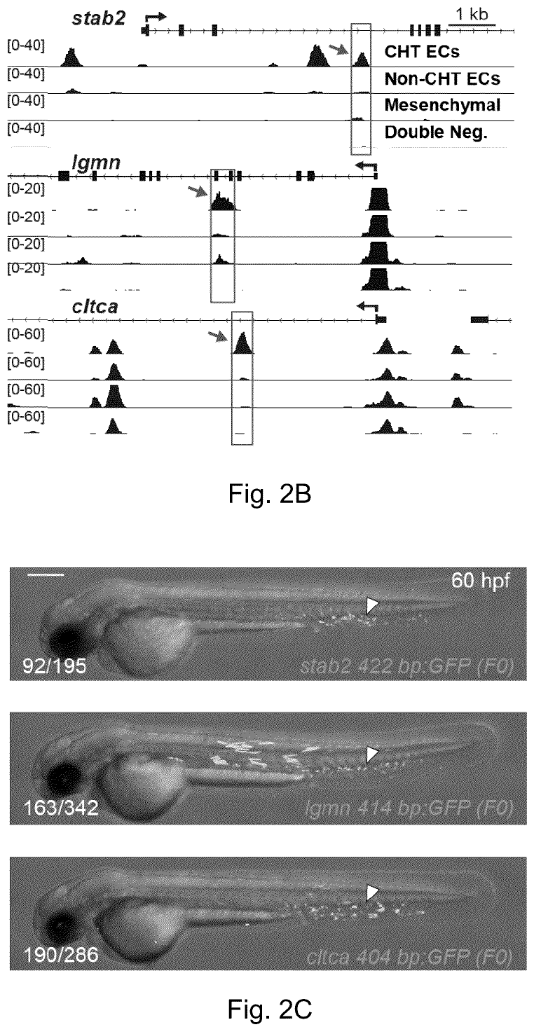

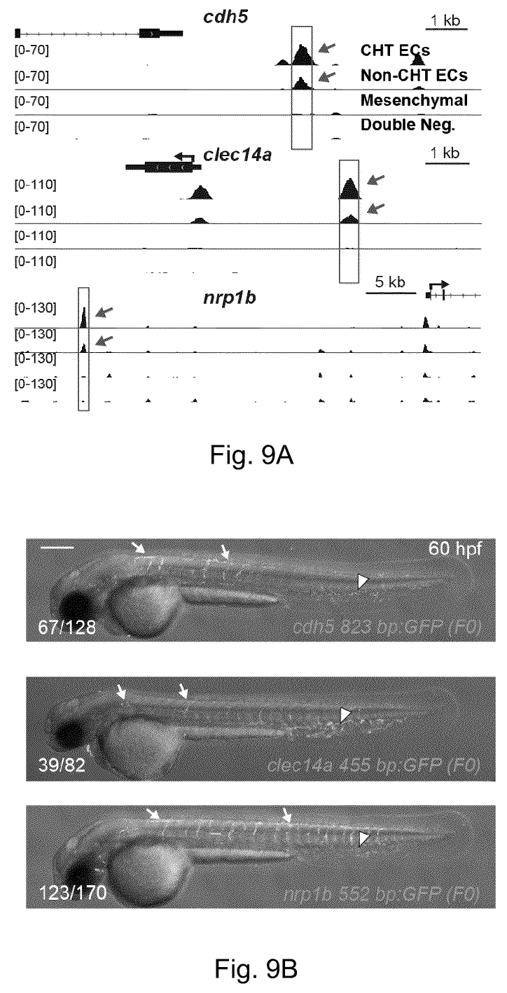

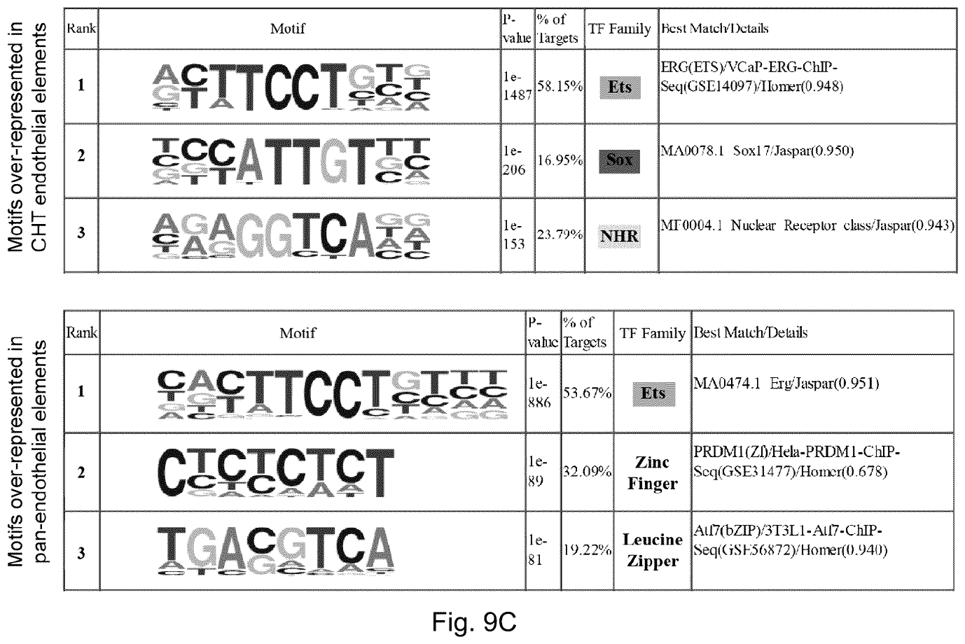

To investigate the transcriptional control of niche-specific gene expression within CHT ECs, double positive mrc1a:GFP/kdrl:mCherry embryos were dissociated and FACS was used to isolate four different populations for RNA-seq and ATAC-seq analyses: GFP⁺/mCherry⁺ (CHT ECs), GFP⁻/mCherry⁺ (ECs outside the CHT), GFP⁺/mCherry⁻ (mesenchymal cells in the tail fin), and GFP⁻/mCherry³¹ (negative remainder of the embryo; see e.g., FIG. 2A). By comparing regions of chromatin accessibility across the four populations, 6,848 regions uniquely open were identified in CHT ECs. Of the 29 CHT EC genes, 26 out of 29 had an ATAC-seq element within 100 kb of the transcriptional start site that was found only in CHT ECs (see e.g., FIG. 2B, Table 1). To test whether these regions might be tissue-specific enhancers, the sequences were cloned for 15 of the elements, which were then fused to a minimal promoter and GFP, and then injected into zebrafish embryos. 12 out of 15 constructs showed GFP expression enriched in CHT ECs at 60-72 hpf (see e.g., FIG. 2C, Table 2). As a control, regions of chromatin were cloned, which were predicted to contain pan-endothelial regulatory elements based on proximity to previously annotated vascular-specific genes and their accessibility in both the CHT and non-CHT EC fractions (see e.g., FIG. 9A-FIG. 9C, Table 2). A number of these regions were previously shown to contain endothelial enhancers (see e.g., Quillien et al. Robust Identification of Developmentally Active Endothelial Enhancers in Zebrafish Using FANS-Assisted ATAC-Seq. Cell reports 20, 709-720, 2017). For 6 out of 6 pan-endothelial regions, mosaic GFP expression in ECs were observed throughout the embryo that was not restricted to the CHT (see e.g., FIG. 9A-FIG. 9C). Thus, the zebrafish system was able to rapidly validate, in vivo, niche-specific endothelial enhancers predicted by ATAC-seq analysis.

### Transcription Factor Binding Sites for Niche Endothelial Expression.

To identify transcription factors that might bind the CHT EC enhancers, a motif enrichment analysis was performed on the 6,848 regions of chromatin that were uniquely accessible in CHT ECs. This analysis revealed that Ets, Sox (SoxF factors, specifically) and Nuclear Hormone Receptor (NR2F2/RORA/RXRA factors, specifically, abbreviated hereafter as NHR) binding motifs were most enriched in the 6,848 regions (see e.g., FIG. 9A-FIG. 9C). In contrast, there were 4,522 pan-endothelial elements (i.e., regions of chromatin accessible in both CHT and non-CHT ECs) across the genome enriched for Ets sites, but not SoxF or NHR binding motifs (see e.g., FIG. 9A-FIG. 9C). Of note, the 12 CHT-EC elements that drove GFP expression in the in vivo reporter assay all harbored Ets, SoxF and NHR sites, whereas one of the three CHT-EC elements that failed to drive GFP expression and three of the six pan-endothelial regions lacked a NHR binding site (see e.g., Table 2).

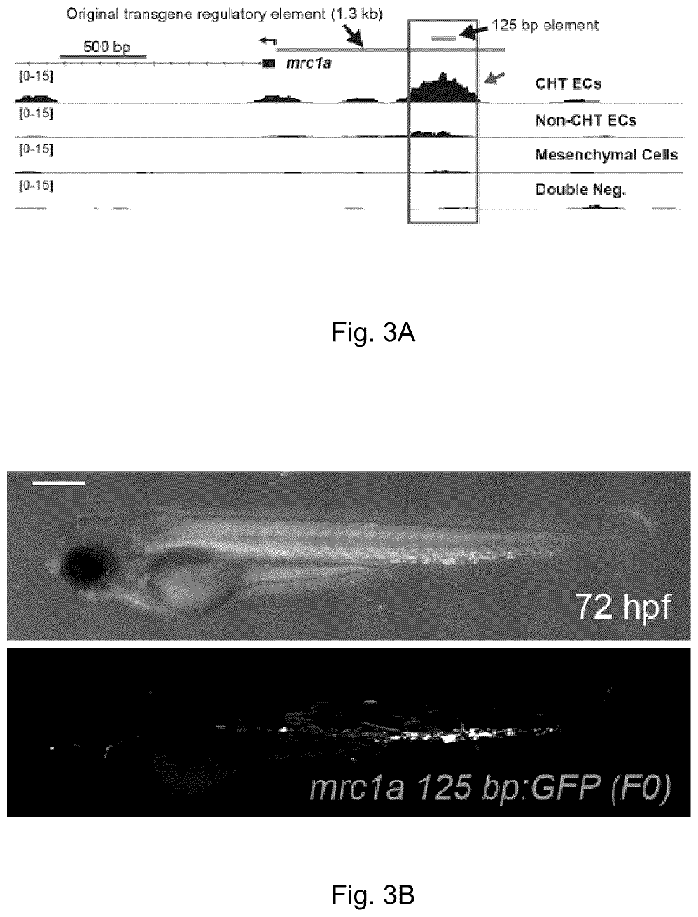

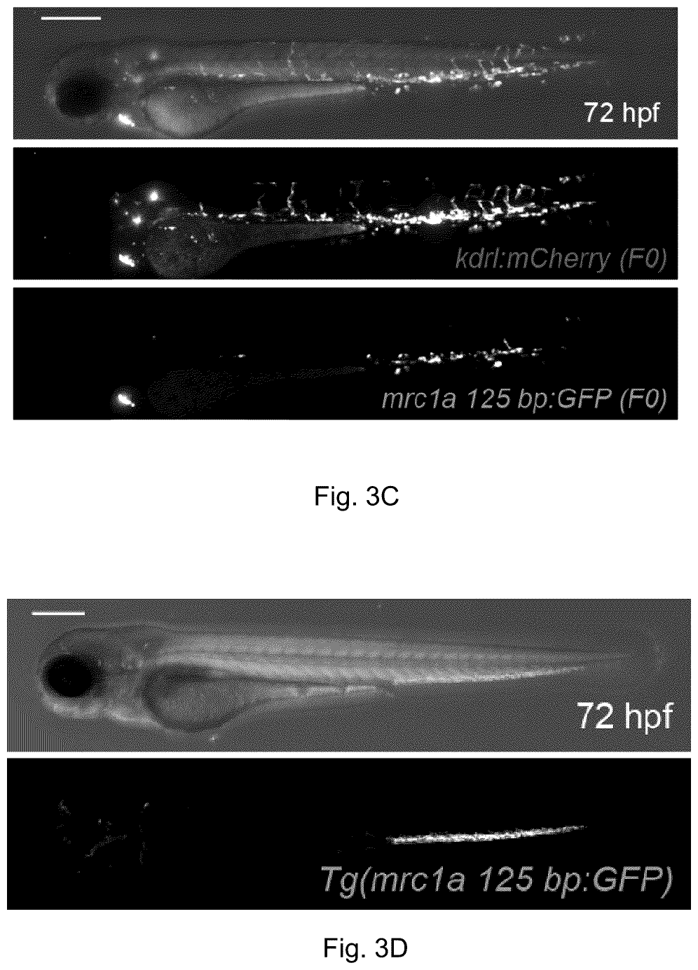

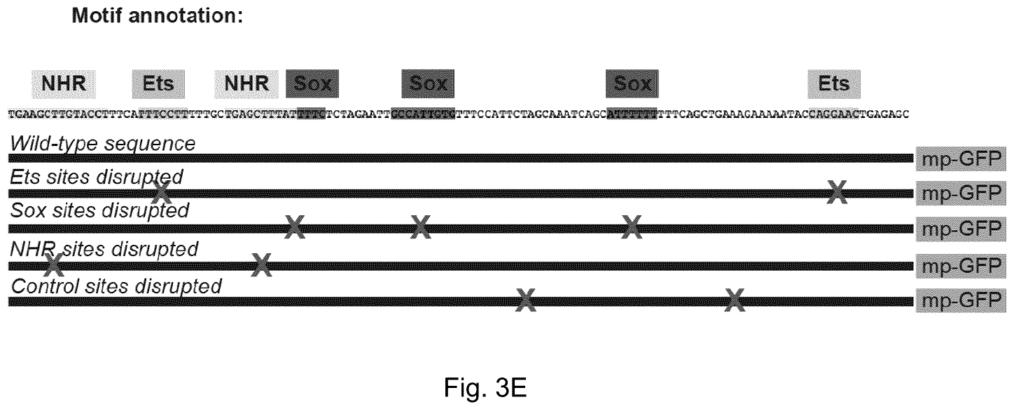

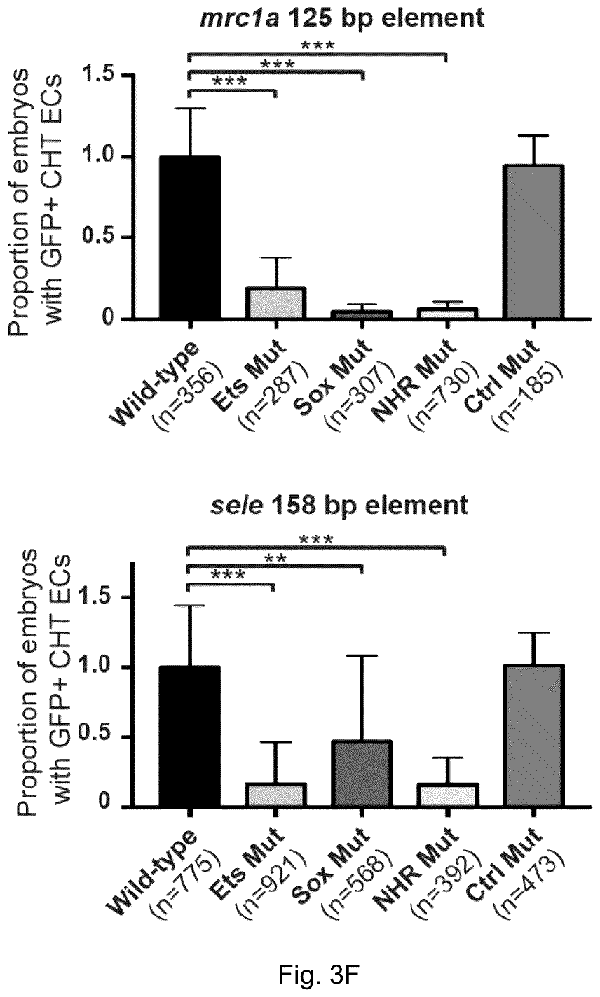

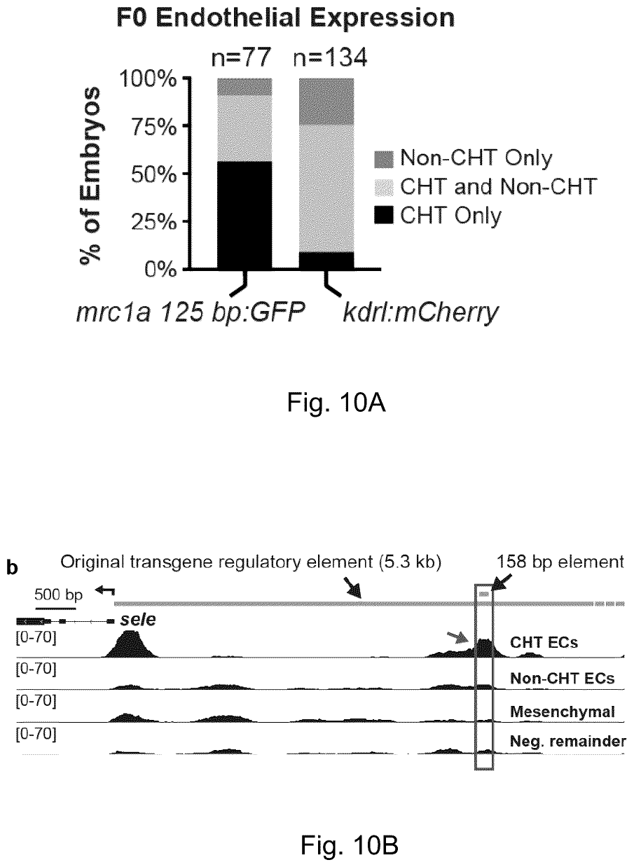

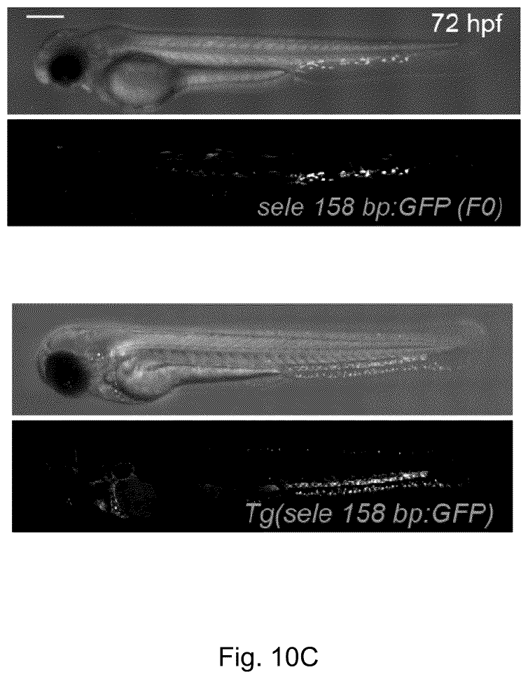

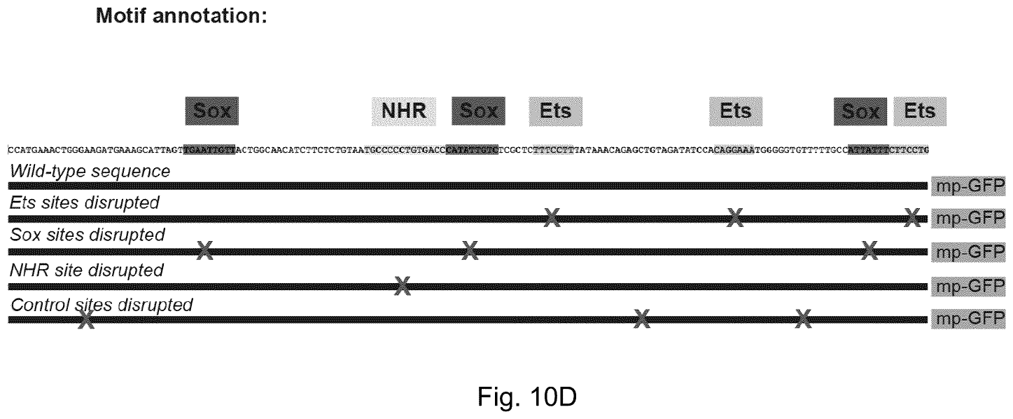

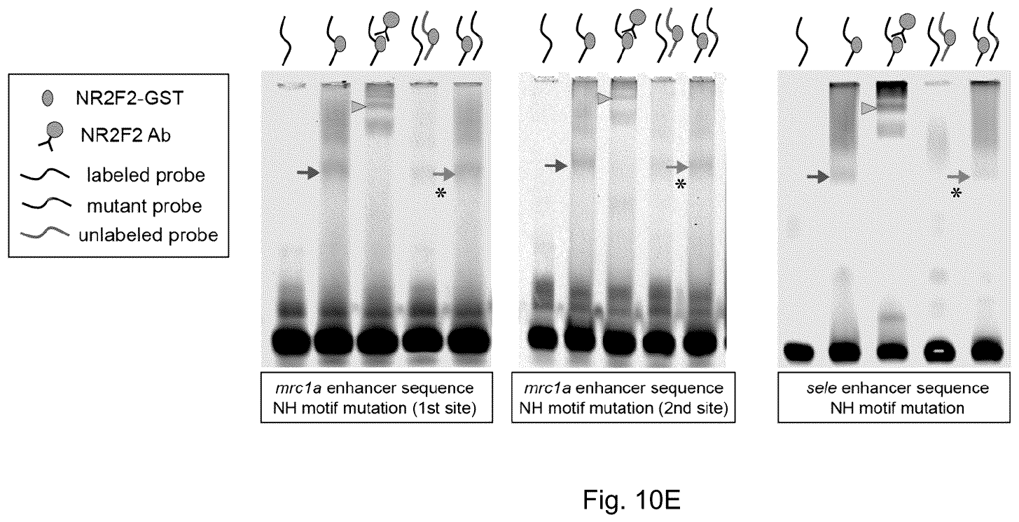

To determine a minimal sequence sufficient to drive CHT EC expression, a 125 base pair (bp) sequence upstream of mrc1a and a 158 bp sequence upstream of sele were cloned; these sequences had been included the our original transgenes and corresponded to the strongest ATAC-seq signal in these regions (see e.g., FIG. 3A, FIG. 10A-FIG. 10E). When coupled to a minimal promoter, these elements drove GFP expression in CHT ECs in 44% (125 bp mrc1a sequence; 155 out of 356) and 23% (158 bp sele sequence; 176 out of 775) of injected embryos (see e.g., FIG. 3B, FIG. 10A-FIG. 10E). Compared to injection of a kdrl: mCherry construct, in which mosaic expression was observed in ECs throughout the embryo, expression of the mrc1a and sele enhancer-GFP constructs was restricted to CHT ECs (see e.g., FIG. 3C, FIG. 10A-FIG. 10E). Moreover, when stable transgenic lines were established using these short constructs, GFP was specifically expressed in CHT ECs (see e.g., FIG. 3D, FIG. 10A-FIG. 10E). Mammalian Mrc1 is prominently expressed by macrophages and venous sinusoidal ECs. Zebrafish have two homologous genes, mrc1a and mrc1b. A recent study of mrc1a reported expression in both macrophages and ECs when the promoter was coupled to an intronic enhancer that showed conservation with mrc1b (see e.g., Jung et al. Development of the larval lymphatic system in zebrafish. Development 144, 2070-2081, 2017). The 125 bp enhancer element, in contrast, drove expression specifically in CHT ECs, illustrating the specificity in the enhancers.

Both the 125 bp mrc1a and 158 bp sele regulatory sequences contained Ets, SoxF and NHR motifs (see e.g., FIG. 3E, FIG. 10A-FIG. 10E). To test whether these transcription factor binding sites were required for expression, variants were generated in which each class of motif was disrupted by mutation. In each case, disruption of the Ets, SoxF or NHR motifs led to a significant reduction or complete loss of GFP expression in CHT ECs (see e.g., FIG. 3F). In control constructs where mutations were targeted to intervening sequences between the Ets, SoxF and NHR motifs, GFP expression was unperturbed (see e.g., FIG. 3F). Studies of arterial-venous specification in zebrafish have shown that the NHR Nr2f2, also known as COUP-TFII, promotes venous endothelial cell fate (see e.g., Aranguren et al. Transcription factor COUP-TFII is indispensable for venous and lymphatic development in zebrafish and Xenopus laevis. Biochemical and biophysical research communications 410, 121-126, 2011). To test whether Nr2f2 could directly bind the enhancer sequences, an in vitro gel electrophoretic mobility shift assay was performed. Incubation of murine NR2F2-GST protein with labeled probe from either the 125 bp mrc1a or 158 bp sele zebrafish enhancers led to DNA:protein complexes that were super-shifted upon addition of an NR2F2 antibody and could be outcompeted by an unlabeled competitor probe (see e.g., FIG. 10A-FIG. 10E). However, unlabeled probes in which the NHR motifs were mutated were unable to outcompete the wild-type probes, demonstrating that NR2F2 binds these NHR motifs in a sequence-specific manner.

### Defined Factors Induce Niche Endothelial Expression.

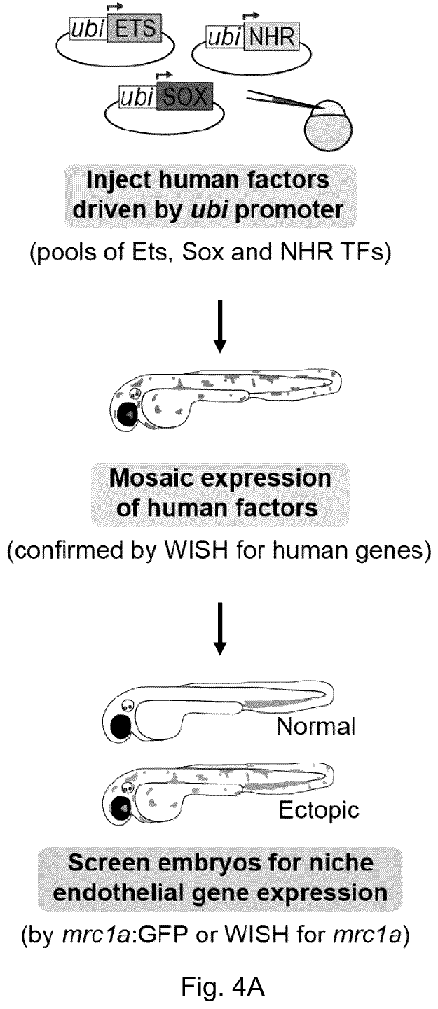

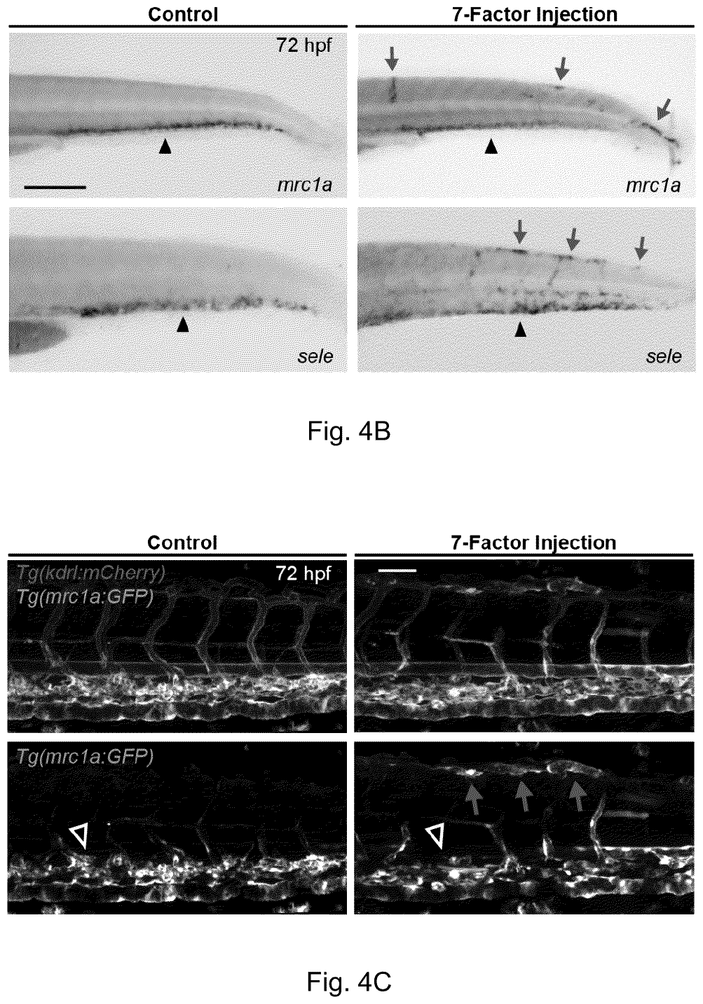

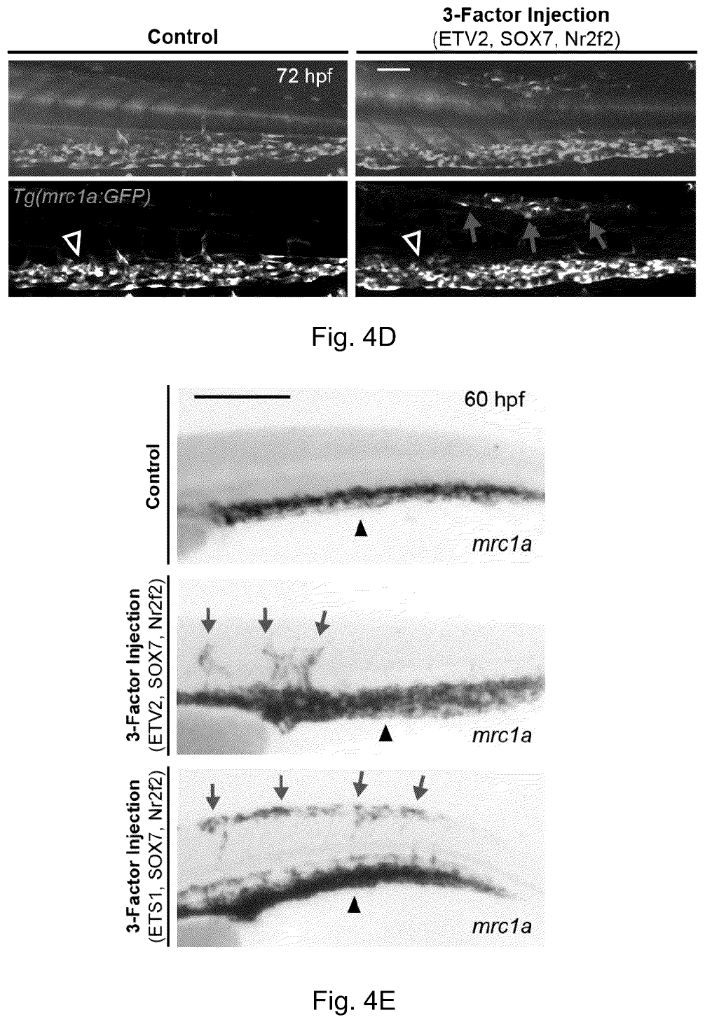

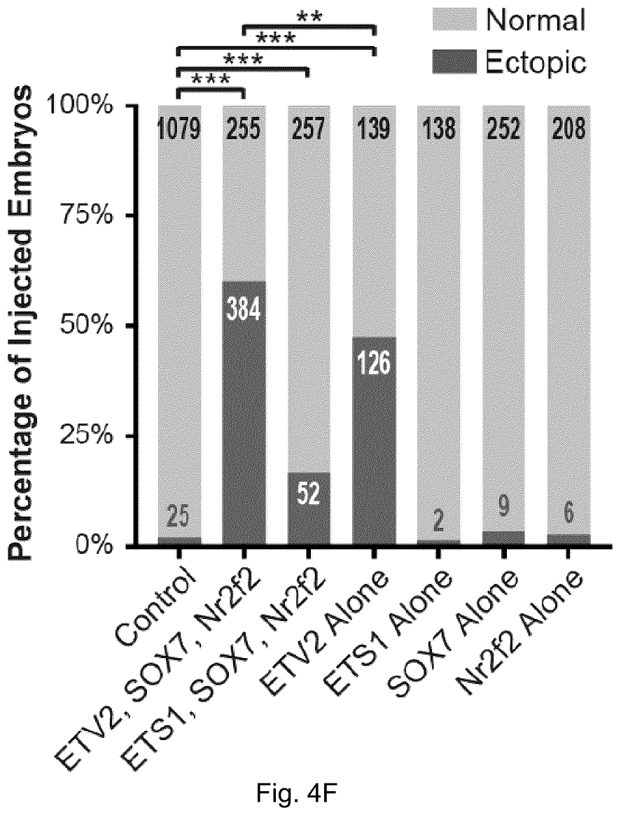

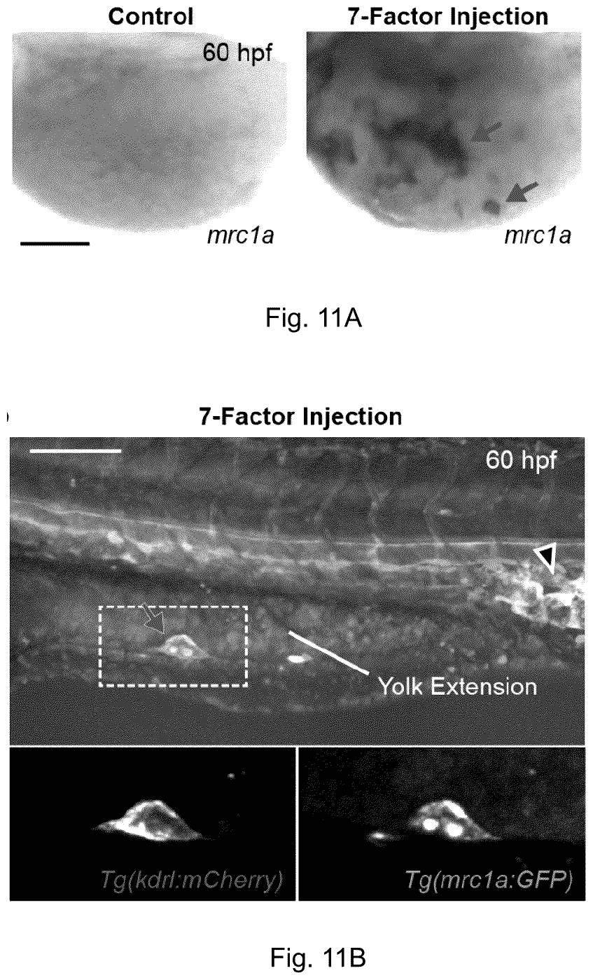

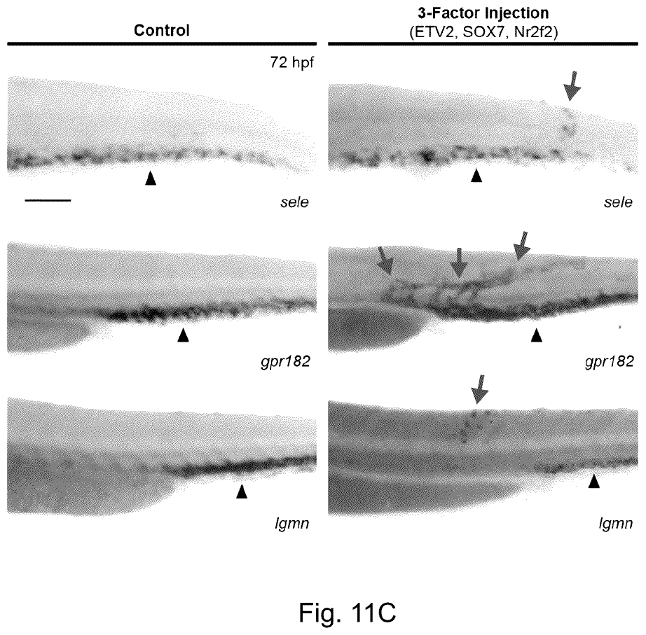

To determine which transcription factors are expressed in CHT ECs and might bind the Ets, Sox and NHR motifs in vivo, RNA-seq data from CHT ECs was examined. The most highly expressed factors were fli1a, etv2, ets1, sox18, sox7, nr2f2 and rxraa (see e.g., Table 4). To test whether these seven factors could induce niche endothelial gene expression outside the CHT, constructs were generated in which the orthologs for each transcription factor was under the control of a ubiquitous (ubi) promoter (see e.g., FIG. 4A). A pool of the seven ubi-driven factors were then injected into one cell-stage zebrafish embryos, and mrc1a and sele expression was examined by WISH at 60-72 hpf. Strikingly, 17% (12 out of 69) of these embryos had ectopic vascular patches of mrc1a expression outside of the CHT, dorsally within the trunk and tail, and over the yolk (see e.g., FIG. 4B, FIG. 11A-FIG. 11C). Control-injected embryos did not show ectopic expression (0 out of 56). Similar results were obtained with WISH for sele or when factors were injected into mrc1a:GFP/kdrl:mCherry double transgenic embryos (see e.g., FIG. 4B-FIG. 4C, FIG. 11A-FIG. 11C). Vessels ectopically expressing mrc1a were often larger than their normal counterparts in these regions and had a sinusoidal-like morphology similar to the CHT (see e.g., FIG. 4C). Using DIC microscopy, blood flow through these regions was readily visualized (data not shown). These data suggest that a small number of transcription factors are sufficient to ectopically induce the niche endothelial gene program.

This mutational analysis of the 125 bp mrc1a and 158 bp sele enhancers indicated that a factor from each of the Ets, Sox and NHR families was required for expression, which led to the experiment of whether a combination of just three factors (one from each family) was sufficient to induce niche endothelial gene expression. ETV2 is a pioneer factor that is essential for specification of early mesodermal progenitors into vascular cell fates. Forced expression of ETV2 in nonvascular cells induces reprogramming towards an early endothelial fate that can generate many types of vasculature. Previous work in zebrafish has shown the importance of SoxF factors (sox7 and sox18) and nr2f2 during arterial-venous specification (see e.g., Swift et al. SoxF factors and Notch regulate nr2f2 gene expression during venous differentiation in zebrafish. Developmental biology 390, 116-125, 2014). A combination of three of these factors—ETV2, SOX7 and Nr2f2—could be sufficient to induce ectopic niche endothelial gene expression. Consistent with this, when these three factors were injected, significant ectopic mrc1a expression was observed (see e.g., FIG. 4D-FIG. 4F). The frequency of ectopic vessels generated with three factors was higher than the 7-factor pool, suggesting factor concentration has functional significance. Injected embryos similarly showed ectopic expression of sele, gpr182 and lgmn, indicating that the 3-factor pool induced the niche endothelial program (see e.g., FIG. 11A-FIG. 11C).

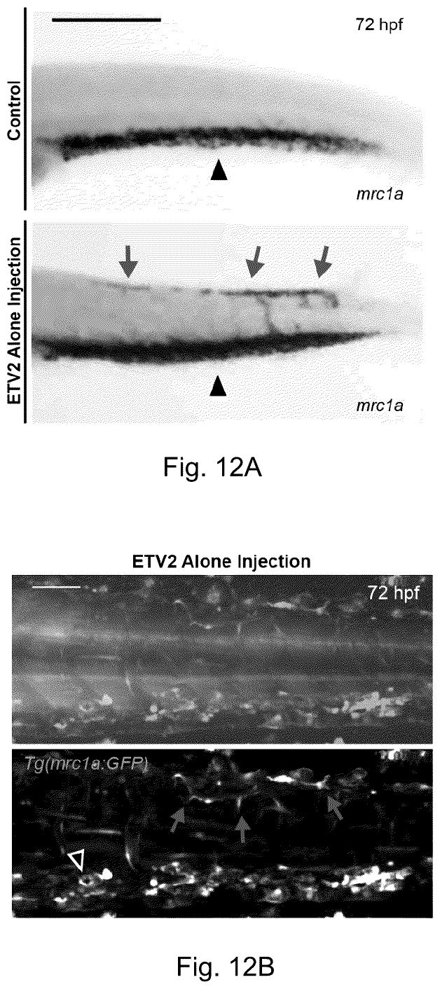

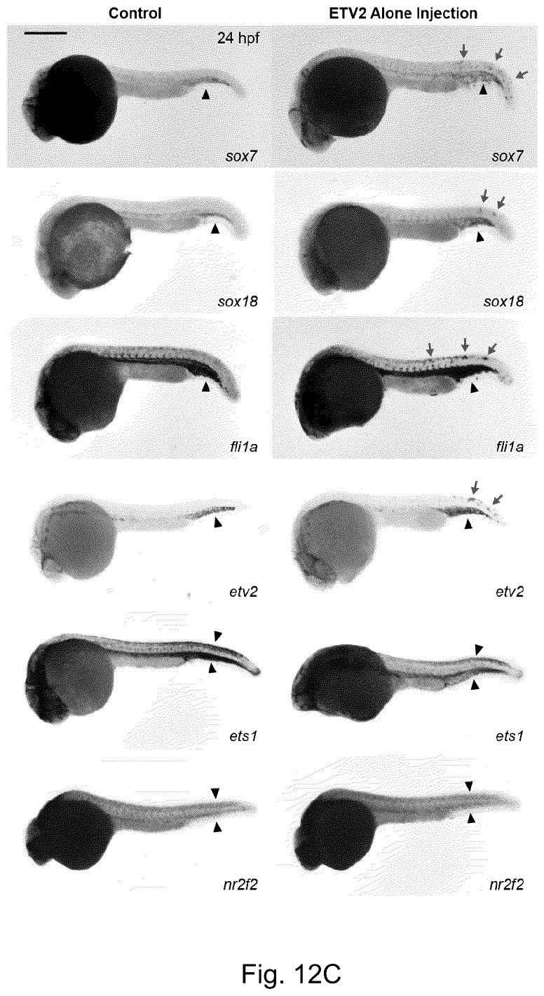

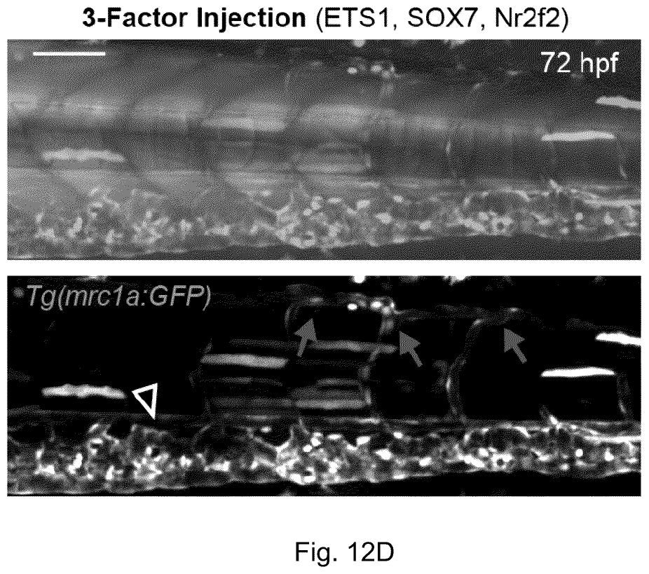

To evaluate the contribution of the individual transcription factors, each factor was injected alone, and WISH was performed for mrc1a. Embryos injected with SOX7 or Nr2f2 alone showed minimal ectopic expression (see e.g., FIG. 4F). Injection of ETV2 alone led to ectopic expression of mrc1a, although at a lower frequency than when ETV2 was injected in combination with SOX7 and Nr2f2, indicating the additional factors increase ectopic induction (see e.g., FIG. 4F, FIG. 12-FIG. 12D). Injecting ETV2 alone induced ectopic expression of the endogenous zebrafish sox7 gene, as well as sox18, fli1a and etv2, demonstrating that human ETV2 can induce several zebrafish endothelial gene programs, including artery, vein and niche endothelial genes (see e.g., FIG. 12-FIG. 12D). Because endogenous ets1 and nr2f2 are expressed broadly outside the CHT (e.g. in the spinal cord region), it was difficult to visualize whether they were similarly induced by ETV2 overexpression (see e.g., FIG. 12-FIG. 12D). ETS1 has the capacity to bind to the consensus ETS motif enriched in the peaks and is expressed at an appreciable level in the niche ECs. In contrast to ETV2, overexpression of ETS1 alone did not lead to widespread vessel induction or ectopic mrc1a expression (see e.g., FIG. 4F). Injection of ETS1, SOX7 and Nr2f2, however, led to significant ectopic expression of mrc1a, illustrating the combinatorial activity of these transcription factors (see e.g., FIG. 4E, FIG. 4F, FIG. 12-FIG. 12D). Notably, the zebrafish genes encoding each of the seven initial transcription factors had regions of chromatin associated with them that were uniquely accessible in the CHT EC fraction and harbored Ets, SoxF and NHR sites (see e.g., Table 5), indicating that these factors regulate one another similar to an auto-regulatory loop established during reprogramming. Thus, overexpression of the three factor combinations, including either ETV2 or ETS1 with Sox and NHR factors, mimics the endogenous expression of these factors in niche ECs at 72 hpf and drives robust specification of the niche endothelial gene program.

### Ectopic Vascular Regions Recruit HSPCs.

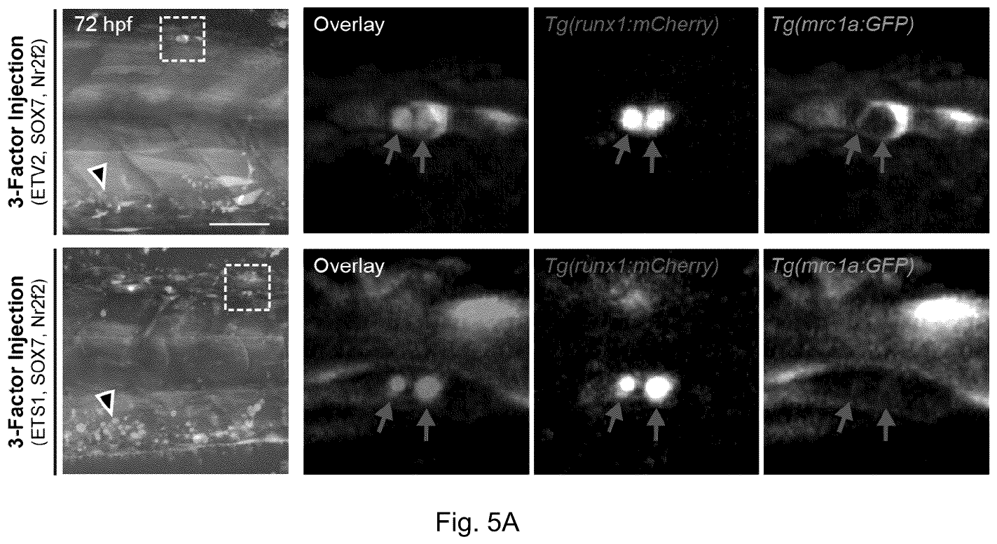

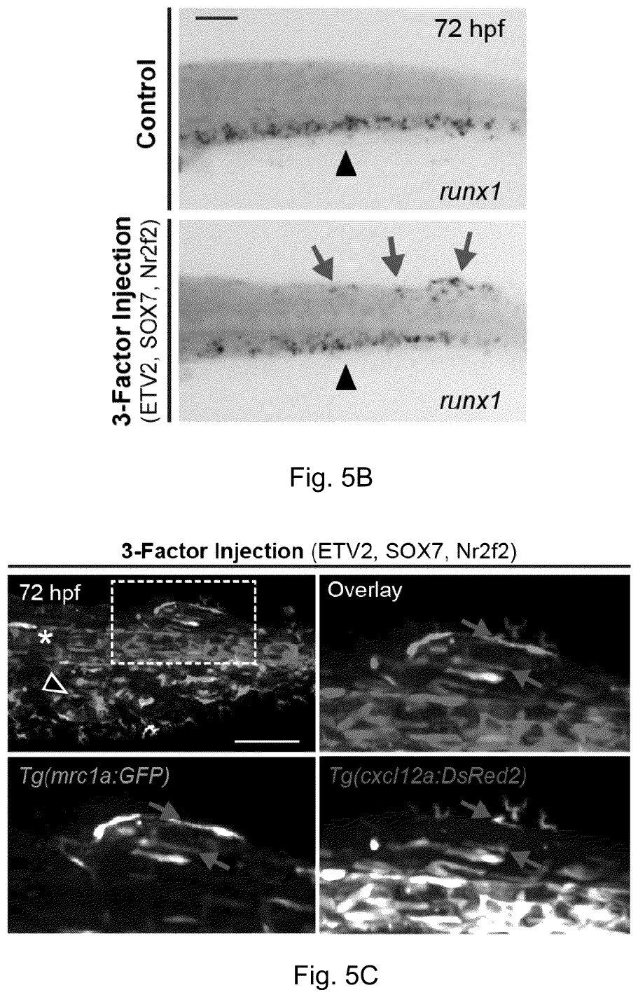

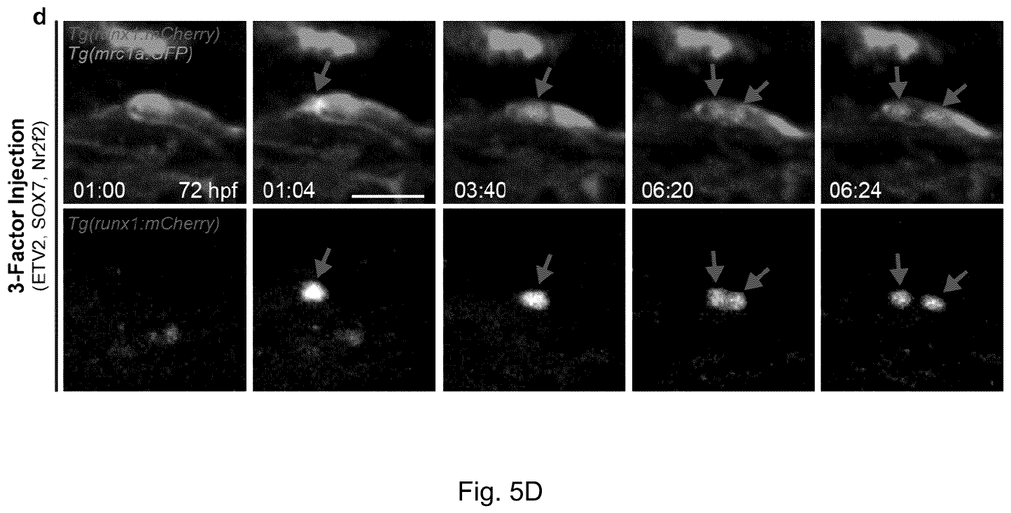

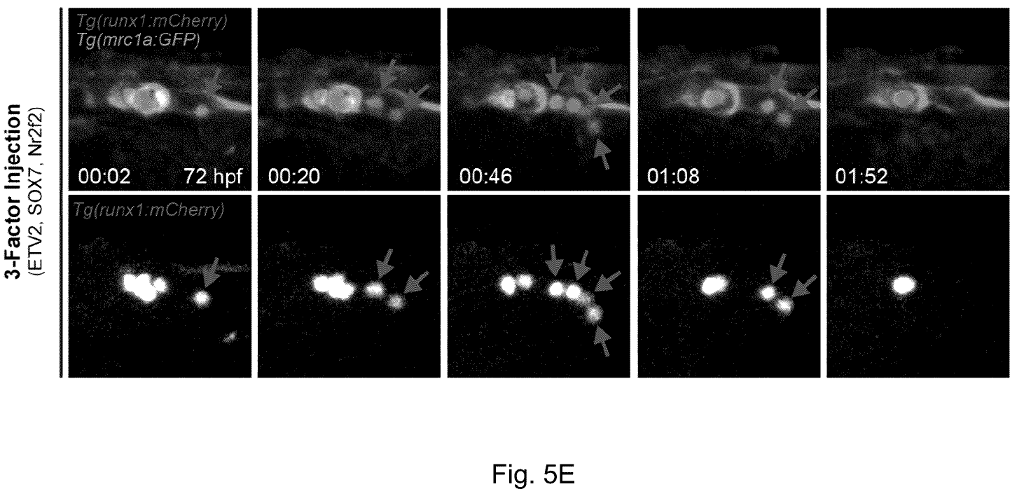

It was next asked whether the ectopic regions of CHT EC gene expression were capable of recruiting and supporting HSPCs. The ETV2, SOX7 and Nr2f2 pool was injected into mrc1a:GFP/runx1:mCherry double positive transgenic embryos, and the localization of runx1:mCherry⁺ HSPCs was examined. Strikingly, in 12 out of 22 embryos that had ectopic vascular patches of mrc1a:GFP, HSPCs were observed localizing to these regions (see e.g., FIG. 5A). In contrast, 0 out of 27 control embryos had ectopically localized HSPCs. Similar ectopic localization of runx1⁺ HSPCs was observed by runx1 WISH and in embryos injected with the ETS1, SOX7 and Nr2f2 combination (see e.g., FIG. 5A, FIG. 5B).

To evaluate whether these ectopic regions provide a supportive environment for HSPCs, high-resolution live cell confocal microscopy was used to more closely examine HSPC behaviors and interactions with ECs at these sites. This analysis revealed that HSPCs directly associated with the mrc1a:GFP⁺ ECs within ectopic sites and could be found in both intraluminal and extravascular spaces. The ectopic mrc1a:GFP⁺ ECs were often associated with cxcl12a:DsRed2⁺ stromal cells and formed pockets around the HSPCs, similar to what is observed in the CHT (see e.g., FIG. 5A, FIG. 5C). Notably, using time-lapse microscopy, the initial recruitment, lodging and division of HSPCs was observed within these sites (see e.g., FIG. 5D). When the HSPCs divided, daughter cells migrated away from the ectopic site and entered circulation, presumably traveling to subsequent niches (see e.g., FIG. 5E). Together, these data demonstrate that reprogramming by the three factors that drive the niche endothelial gene program leads to functional ectopic niches that can recruit and sustain HSPCs.

### A Conserved Endothelial Signature in the HSPC Niche.

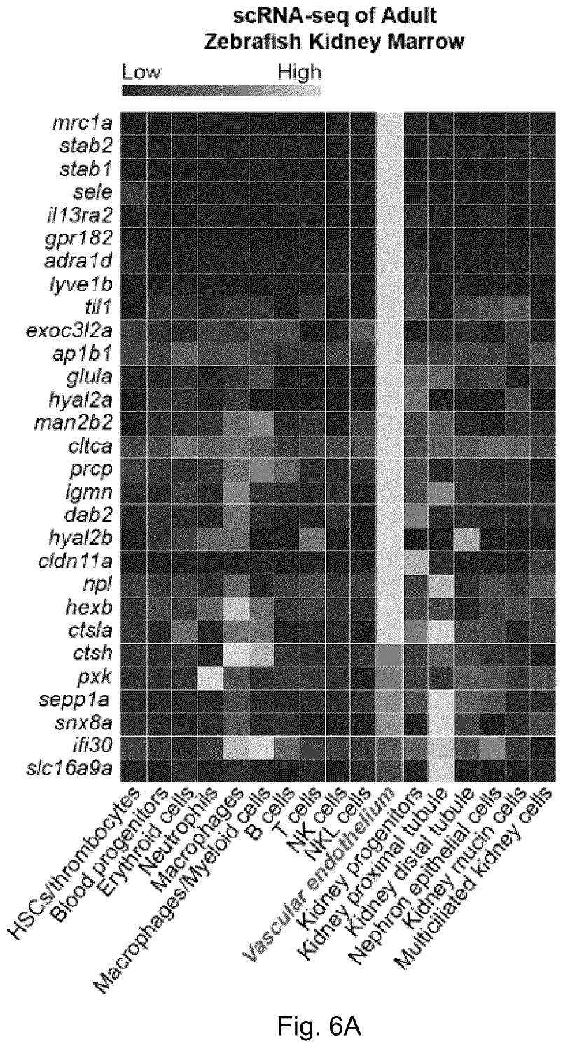

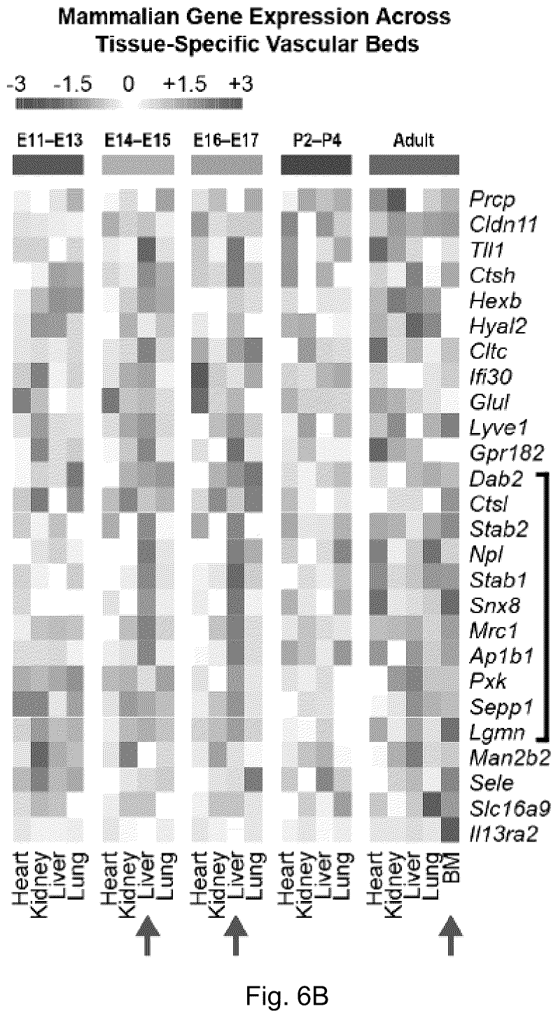

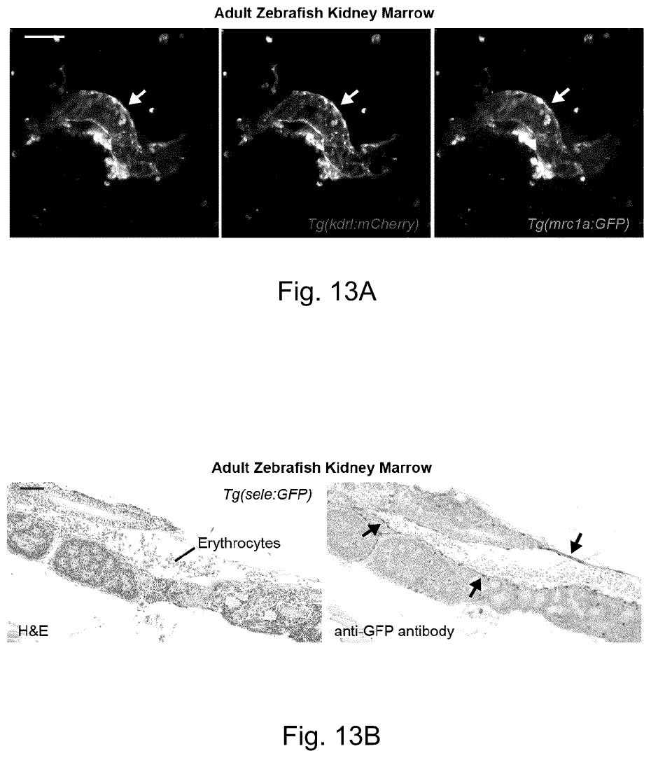

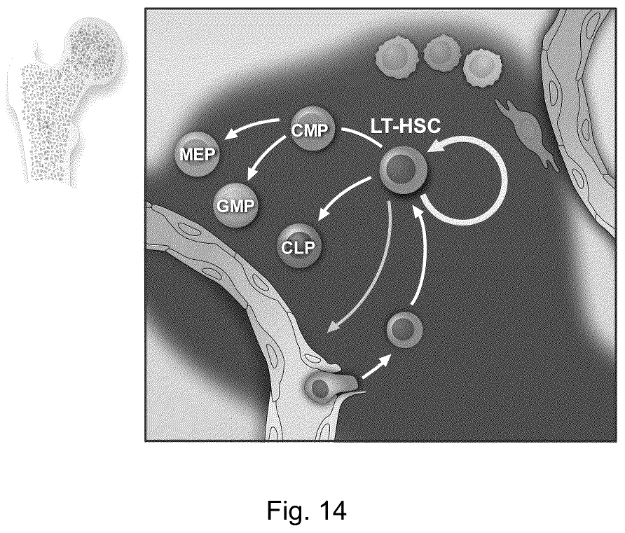

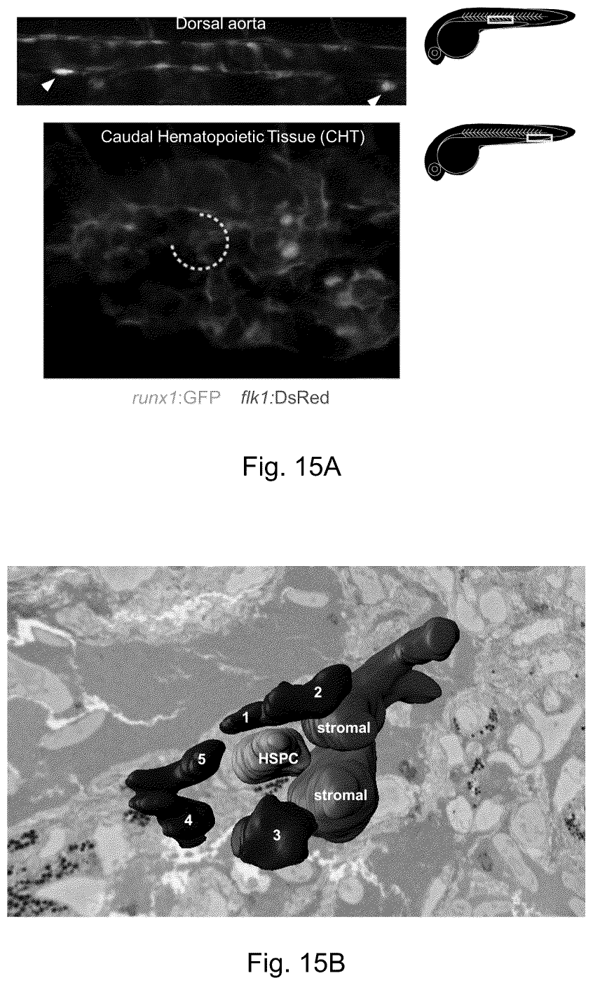

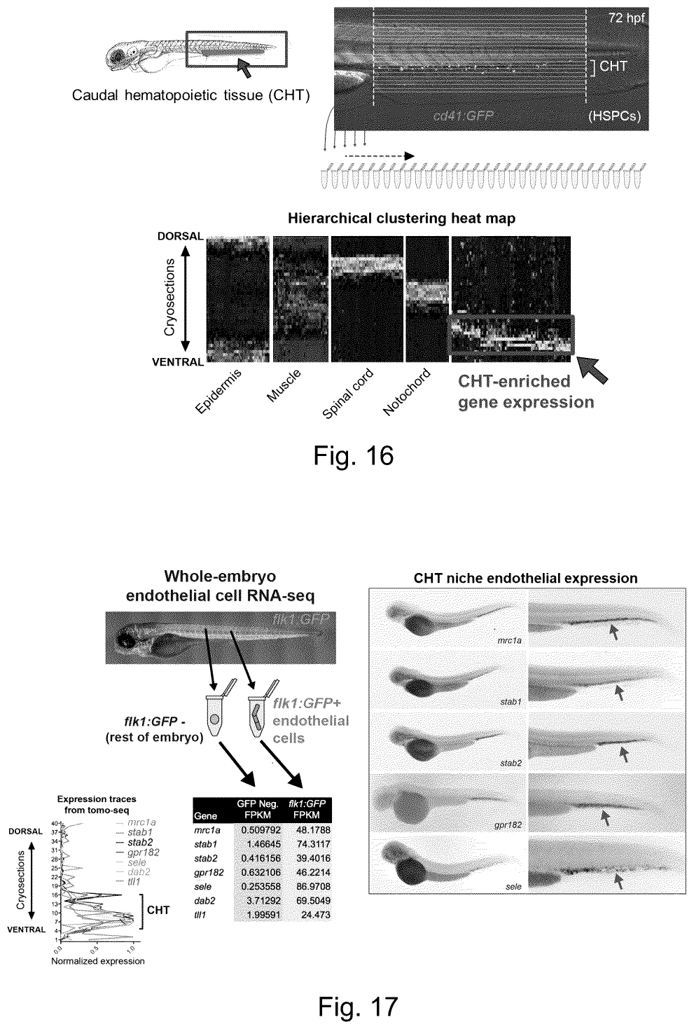

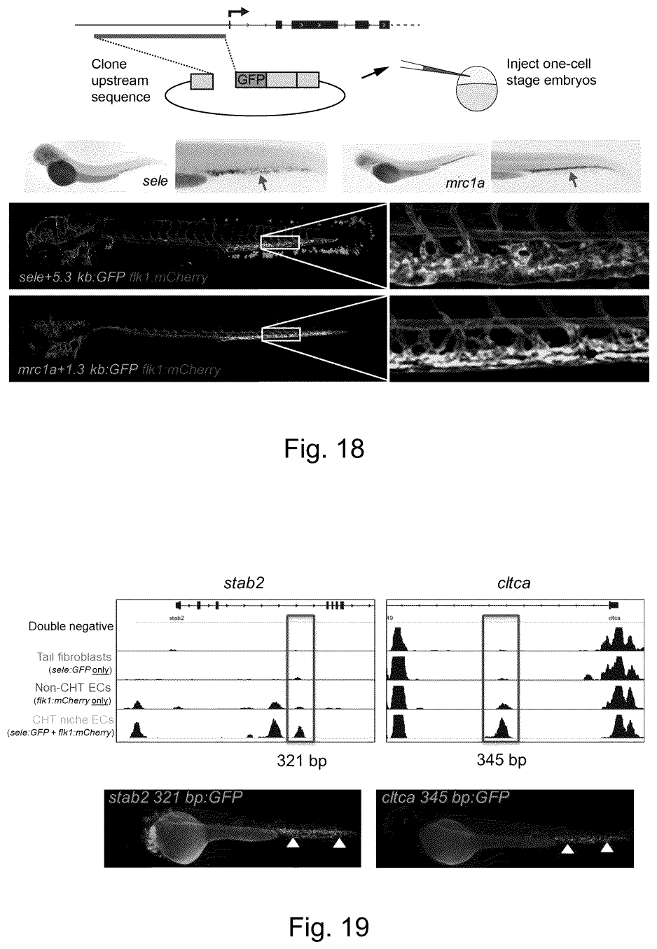

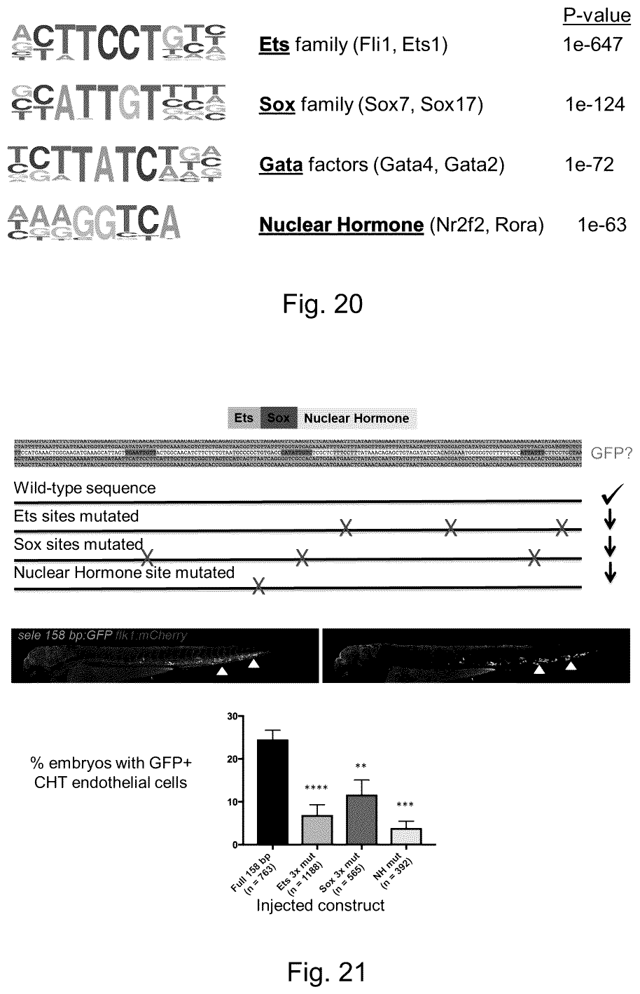

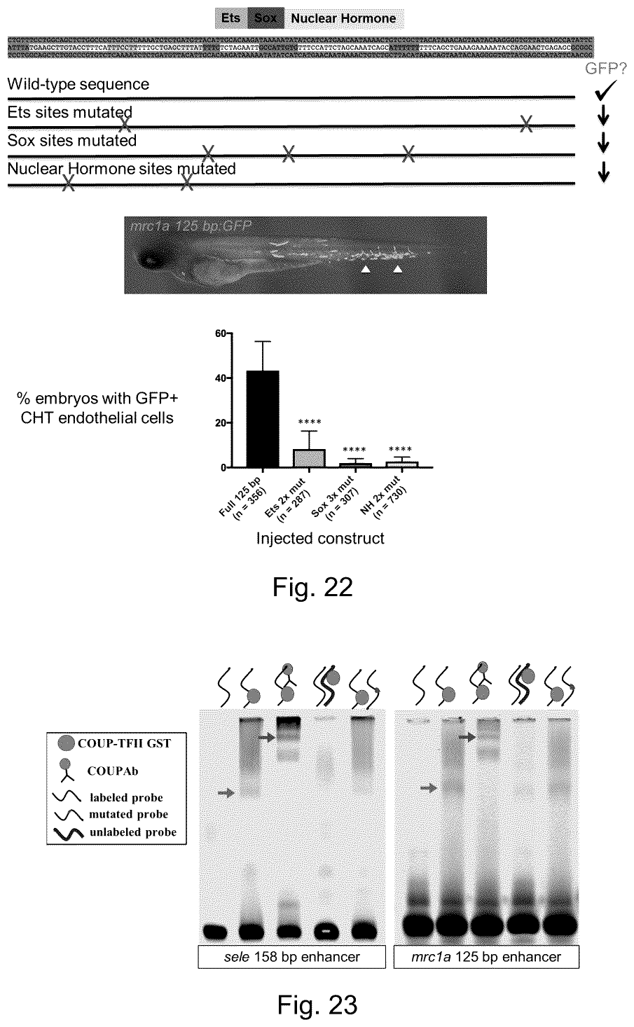

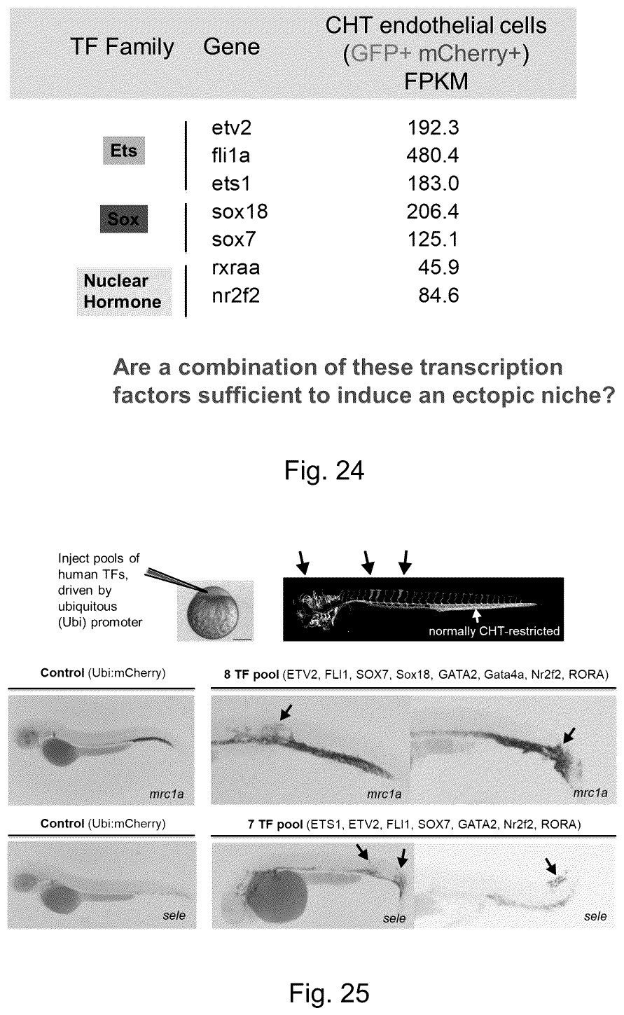

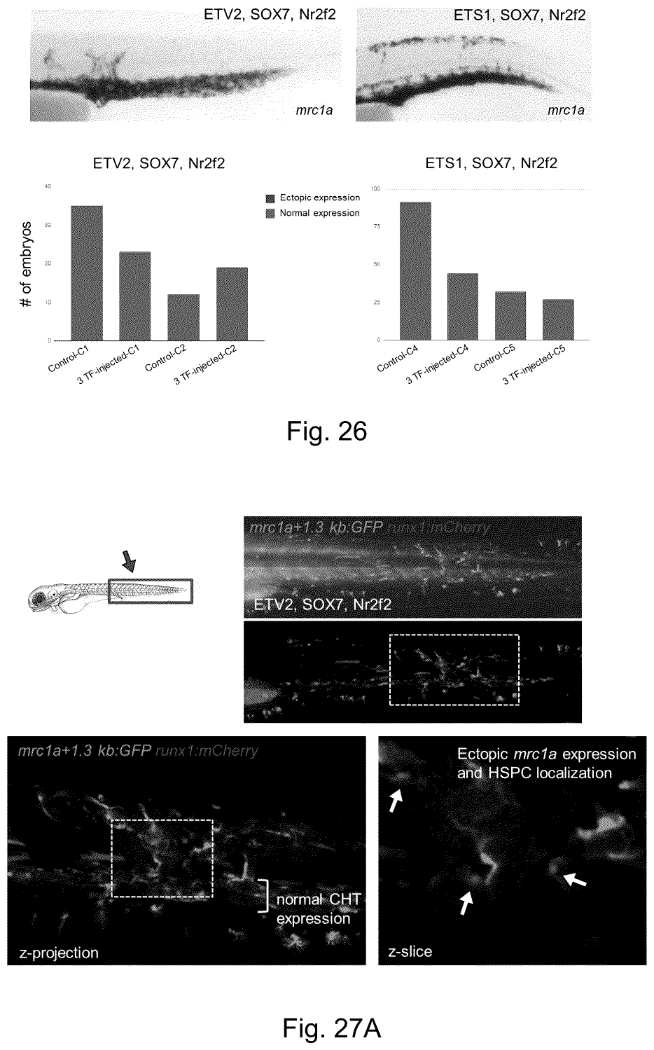

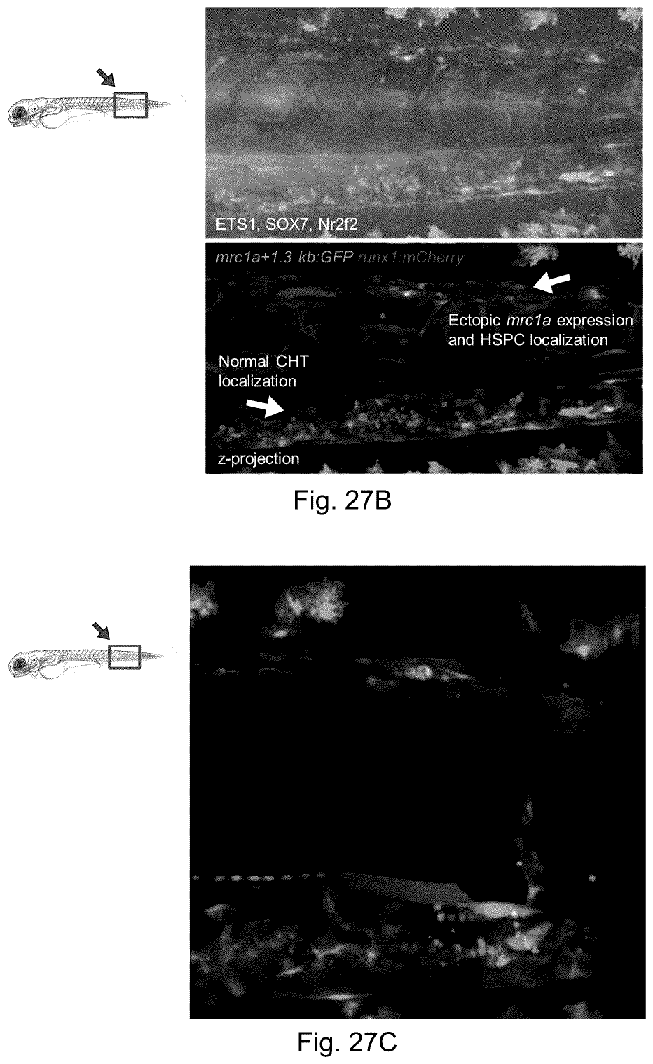

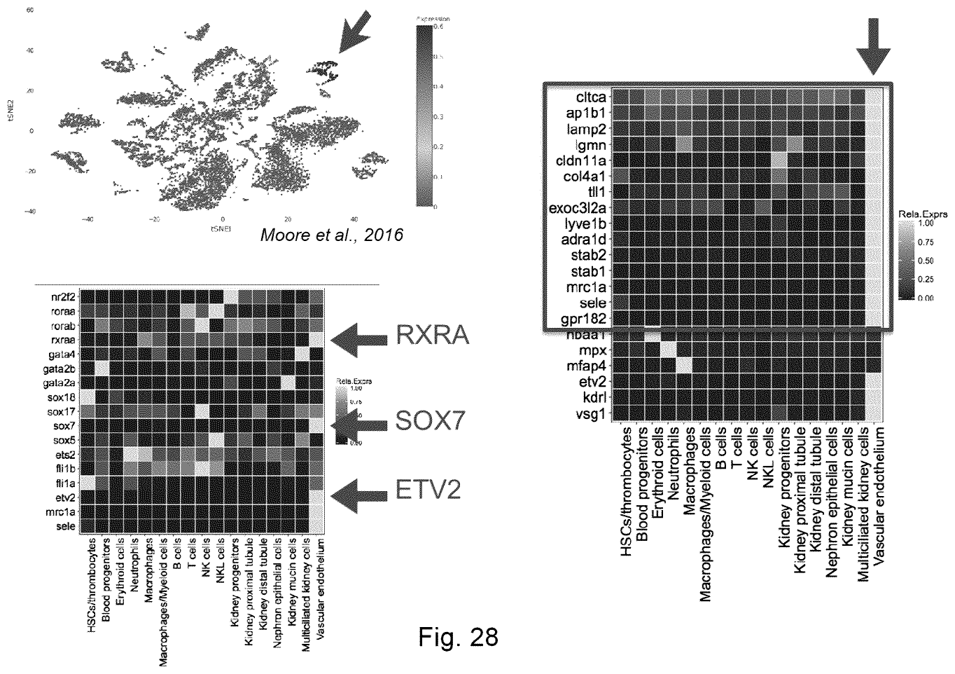

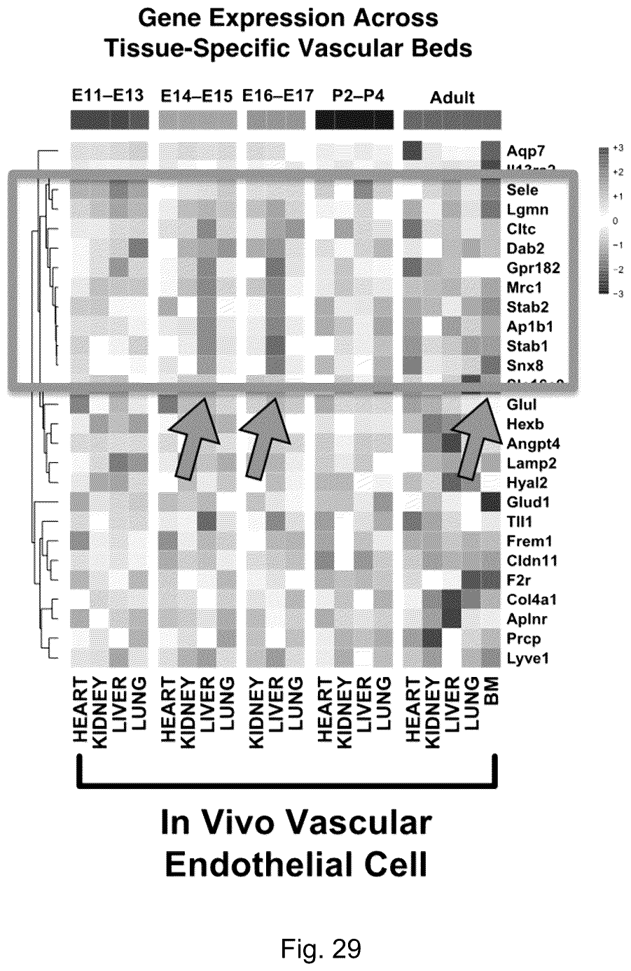

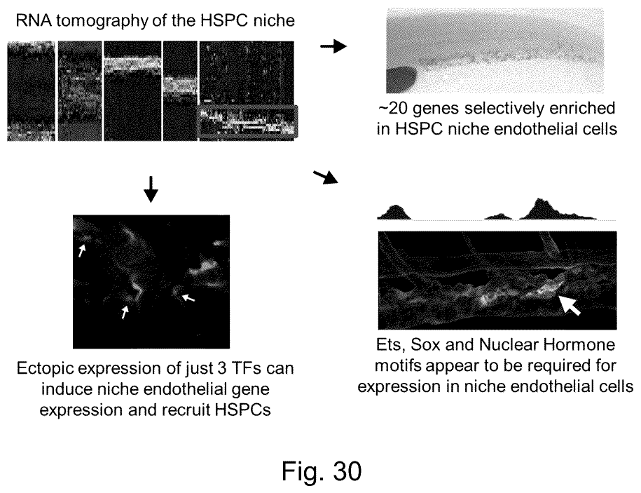

To determine whether a similar endothelial expression signature was present in the adult zebrafish niche, the mrc1a:GFP and sele:GFP transgenes were examined, and it was found that both lines had GFP expression in ECs in the kidney marrow (see e.g., FIG. 13A, FIG. 13B). In addition, a single cell RNA-seq dataset for zebrafish kidney marrow was examined (see e.g., Tang et al. Dissecting hematopoietic and renal cell heterogeneity in adult zebrafish at single-cell resolution using RNA sequencing. The Journal of experimental medicine 214, 2875-2887, 2017). This analysis found that 23 out of 29 of the CHT EC genes were strongly enriched in vascular ECs in the kidney marrow (see e.g., FIG. 6A). To determine whether this pro-haematopoietic vascular niche signature is conserved in mammals, an RNA-seq dataset was probed, which comprises gene expression for ECs from multiple organs of the mouse, including the heart, kidney, liver, lung and bone marrow, from five stages of development (E11-13, E14-15, E16-17, P2-P4 and adult). Orthologs for 21 out of 29 CHT EC genes were enriched in the ECs of a mammalian haematopoietic organ—the fetal liver and/or adult bone marrow—relative to their expression in ECs from non-haematopoietic organs at the same stage of development (see e.g., FIG. 6B). Notably, a subset of expression patterns mirrored the temporal dynamics of HSPC ontogeny, showing robust expression in fetal liver ECs at the E14-17 stages and then later displaying elevated expression in adult bone marrow ECs with a concomitant and expected reduction in liver EC expression (P2-P4 and adult; see e.g., FIG. 6B). To determine if a transcriptional program similar to the one uncovered in the zebrafish could control these genes in the mouse, the expression of transcription factors from the Ets, Sox and NHR families was examined using the same RNA-seq datasets. In both fetal liver ECs (E14-17) and adult bone marrow ECs, the most highly expressed factors were Ets1, Sox 18 and Nr2f2 (see e.g., Table 5). Taken together, these data indicate that a conserved transcriptional program regulates the pro-haematopoietic niche identity of ECs in the fetal and adult HSPC niches.

### Discussion

These data support a model in which a transcriptional program comprised of factors from the Ets, SoxF and NHR families specifies the identity and unique capacity of vascular niche ECs to maintain and expand blood stem cells. This is a conserved feature of the haematopoietic niche at multiple stages of development and across species. The niche endothelial expression signature identified herein comprises genes with known niche functions (e.g., the adhesion receptor E-selectin and the cysteine proteinase Cathepsin L, which has been linked to Cxcl12 turnover in the bone marrow). In addition, there are numerous genes not previously associated with the HSPC niche, including several genes with scavenger functions or activities related to endocytosis and intracellular uptake, including mrc1a, stab½, dab2, ap1b1, pxk and snx8. These molecules could regulate ligand and receptor turnover in the niche or could function to clear potentially harmful agents, such as waste products, modified proteins, or viral, bacterial or fungal related material, from the niche microenvironment. CHT ECs share gene expression with lymphatic ECs, including genes such as lyve1b, which is consistent with their shared origin. In a recent study of the mouse bone marrow, differential gene expression was examined between SECs and AECs. The niche EC signature disclosed herein aligns with the venous SECs of the marrow. Although AECs may also support haematopoiesis, the work herein illustrates the sufficiency of SECs to instruct vascular niche formation and foster stem cell expansion. Extramedullary haematopoiesis that occurs during stress could involve the local induction of this SEC niche program. Other transcription factors have been shown to drive CHT gene expression that maintains haematopoietic cells, including tfec and klf6a, although enriched binding motifs were not observed for these factors. It is plausible that their targets are in different cells of the CHT or that these factors act downstream of our program to specify diverse populations of tissue-specific vascular niches.

Experiments with parabiotic mice have indicated that the size of the niche determines the number of HSPCs (see e.g., Chen et al. Mobilization as a preparative regimen for hematopoietic stem cell transplantation. Blood 107, 3764-3771, 2006). Functional ectopic niches, termed ossicles, have been used to assemble a bone marrow equivalent when transplanted into mice, and it is likely that SECs are present in these structures. Together, these studies and the work presented herein support a method of increasing HSPC numbers in vivo by generating ectopic vascular niches at new safe harbor locations in the body. This approach lays the foundation for novel treatments for diseases where the endogenous bone marrow niche is compromised (e.g., myelofibrosis). At a broader level, this work advances the fundamental understanding of the vascular niche that choreographs homeostasis and regeneration of blood stem cells, which can guide new therapeutic strategies to culture and expand HSPCs for transplantation.

### Methods

### Animal Models.

Wild-type AB, casper or casper-EKK, and transgenic lines cd41:EGFP, runx1:mCherry [runx1+23:NLS-mCherry], kdrl(flk1):GFP [kdrl:GRCFP], kdrl:mCherry [kdrl:Hsa.hras-mCherry], and cxcl12a(sdf1a):DsRed2 were used in this study. Alternative gene names are listed in parenthesis and full transgene names are listed in brackets.

### Genomic Analyses.

For RNA tomography (tomo-seq), 72 hpf embryos were euthanized by tricaine overdose and the portion of the tail containing the CHT was manually dissected using a scalpel. The tissue was oriented in optimal cutting temperature (OCT) tissue freezing media in a cryomold with the ventral side facing the bottom of the mold. After snap freezing on dry ice, 40 individual 8 μm-thick cryosections were collected along the dorsal-ventral axis using a cryostat. The RNA from individual cryosections was extracted using TRIzol™ and then barcoded during a reverse transcription step prior to pooling for library preparation and sequencing (see e.g., Junker et al. Genome-wide RNA Tomography in the zebrafish embryo. Cell 159, 662-675, 2014). For single cell and bulk RNA-seq, kdrl:GFP embryos were dissociated using Liberase™ and GFP⁺ cells were isolated by FACS. For bulk RNA-seq, total RNA was isolated using TRIzol™ and GenElute LPA™ carrier. Libraries were prepared from 50 ng of total RNA/sample as input using Ribogone™ and a SMARTer Universal Low Input RNA Kit™. For single-cell sequencing, approximately 2,000 cells were encapsulated, and libraries were prepared for sequencing. For ATAC-seq, embryos were dissociated using Liberase™ and a minimum of 12,000 cells (max 50,000) were isolated by FACS. Cells were subsequently lysed and isolated nuclei were incubated in a transposition reaction. All sequencing was done using an Illumina Hiseq 2500™. For RNA-seq, quality control was performed by Fast QC™ and Cutadapt™ to remove adaptor sequences and low quality regions. High-quality reads were aligned to UCSC build danRer7 of the zebrafish genome using Tophat™2.0.1158 without novel splicing form calls. Transcript abundance and differential expression were calculated with Cufflinks™2.2.159. FPKM values were used to normalize and quantify each transcript. For ATAC-seq, reads were aligned to UCSC build danRer7 of the zebrafish genome using Bowtie2™ (version 2.2.1) with the following parameters: —end-to-end, —N0—, —L20. The MACS2™ (version 2.1.0) peak finding algorithm was used to identify regions of ATAC-seq peaks with the following parameters: —nomodel—shift-100—extsize 200. An initial q-value threshold of enrichment of 0.05 was used for peak calling and a more stringent q-value of 14 was used to identify peaks that were distinct between different samples. Genome-wide motif enrichment analysis was performed using HOMER™ and motif annotation was done using Consite™. Gene expression analysis of the adult kidney marrow was performed data available on the world wide web at molpath.shinyapps.io/zebrafishblood/.

### Whole Mount In Situ Hybridization (WISH).

In situ hybridization was performed using a standard protocol. Embryos were subsequently transferred to glycerol for scoring and imaging. In situ probes were generated by PCR amplification using a cDNA or plasmid (for transcription factors from other species) template followed by reverse transcription with digoxigenin-linked nucleotides. Primer sequences for all WISH probes used herein are provided in Table 7. WISH images for the 35 CHT-enriched genes identified by tomo-seq can be found on the world wide web at zfin.org.

### Transgenesis and Enhancer-GFP Reporter Assays.

Transgenic lines were established. For the mrc1a:GFP and sele:GFP transgenes, 1.3 kb and 5.3 kb sequences, respectively, upstream of the transcriptional start site were PCR amplified off of genomic DNA and then TOPO-TA™ cloned into a p5E Gateway™ vector, which was then recombined with GFP and a poly A tail, all flanked by Tol2 sites. For the 125 bp mrc1a and 158 bp sele enhancers, the elements were PCR amplified off of genomic DNA, TOPO-TA™ cloned into a p5E Gateway™ vector and then recombined with the mouse Beta-globin minimal promoter fused to GFP with a polyA tail, all flanked by Tol2 sites. Embryos were injected at the one cell-stage with Tol2 RNA and at least two independent lines showing similar expression were established for each construct: (Tg(mrc1a(1.3 kb):GFP); Tg(sele(5.3 kb):GFP); Tg(mrc1a(125 bp):GFP); and Tg(sele(158 bp):GFP). The CHT EC and pan-EC ATAC-seq elements were similarly amplified by PCR using genomic DNA and then fused to the Beta-globin minimal promoter and GFP. Mutational variants of 125 bp mrc1a and 158 bp sele were generated by annealing overlapping oligos followed by a T4 DNA polymerase reaction to generate blunt-ended products, which were subsequently cloned into p5E Gateway™ vectors (following A-tailing with Klenow Fragment using the same work flow as for the ATAC-seq elements). Transcription factor binding motifs were disrupted by changing nucleotides in the core binding sites, purines for pyrimidines and vice versa. Injected FO embryos were scored between 60-72 hpf. Control and experimental groups were blinded prior to scoring and all experiments were performed at least three times, with independent clutches. GFP expression in CHT ECs or pan-EC expression was scored as significant if it was observed in at least 10% of F0 injected embryos. Embryos scored as negative had either no GFP expression or had only sparse ectopic expression in muscle cells. The sequences for primers used to amplify the mrc1a and sele regulatory elements, as well as the 15 CHT EC and 6 pan-EC ATAC-seq elements, are provided in Table 7. The sequences for the overlapping oligos that were used to generate the enhancer variants are provided in Table 8. The fidelity of all constructs was confirmed by sequencing prior to injection.

### Transcription Factor Overexpression Studies.

For transcription factor overexpression studies, the open reading frames for the human (FLI1, ETV2, ETS1, SOX7 and RXRA), Xenopus (Sox18) or zebrafish (Nr2f2) genes were cloned into a pME Gateway vector (Invitrogen™) and then recombined with the zebrafish ubi promoter and a polyA tail, all flanked by Tol2 sites. The fidelity of all constructs was confirmed by sequencing prior to injection. Embryos were injected with pools of ubi-driven transcription factors (1 nl at 25 ng/μl total DNA, plus Tol2 RNA) at the one cell-stage and then screened between 24-72 hpf for ectopic niche endothelial gene expression or ectopic HSPC localization. For control and single-factor injections, the empty Tol2 Gateway™ destination vector was used as filler DNA in the injection mix. Expression of the transcription factors was confirmed by WISH using species-specific in situ probes. Ectopic expression was scored as vascular staining or vascular GFP expression outside the normal domain of gene expression. Control and experimental samples were blinded prior to scoring and all experiments were performed at least three times, with independent clutches.

### Microscopy and Image Analysis

Time-lapse microscopy was performed using a Yokogawa CSU-XI™ spinning disk mounted on an inverted Nikon Eclipse TiTM microscope equipped with dual Andor iXon™ EMCCD cameras and a climate controlled (maintained at 28.5° C.) motorized x-y stage to facilitate tiling and imaging of multiple specimens simultaneously. Screening of injected enhancer-GFP constructs and imaging of WISH embryos was performed using a Nikon SMZ18™ stereomicroscope equipped with a Nikon DS-Ri2™ camera. All images were acquired using NIS-Elements™ and processed using Imaris™ or Adobe Photoshop™ software. Embryos were mounted for imaging. Briefly, specimens were mounted in 0.8% LMP agarose with tricaine (0.16 mg/ml) in glass bottom 6-well plates and covered with E3 media containing tricaine (0.16 mg/ml).

### Flow Cytometry, Kidney Marrow Dissection, Dissociation and Histology.

Embryos were prepared for FACS. Briefly, embryos were chopped with a razor blade in cold PBS and then incubated in Liberase™ for 20 minutes at 37° C. before filtering the dissociated cells through a 40 μm mesh filter and transferring to 2% FBS. FACS was performed using a FACS Aria™ machine. Gates were set using transgene positive and negative control embryos, and SYTOX Blue™ was used as a live/dead stain. At least 12,000 (50,000 max) cells were collected per sample for ATAC-seq experiments and at least 10,000 (300,000 max) cells per sample were collected for RNA-seq experiments. Kidney marrow was harvested from adult zebrafish by manual dissection and then fixed in 4% PFA (for histology) or dissociated by gentle pipetting (for live cell imaging). For histology the kidney marrow was embedded in paraffin prior to sectioning; alternating sections were stained with H&E or with an antibody to GFP. Mouse EC populations were sorted as Cd45⁻Pdpn⁻Cd31³⁰ cells.

### Electrophoretic Mobility Shift Assay

The Nr2f2 fragment was cloned into the pGEX2TK™ vector to generate GST-tagged Nr2f2 and fidelity was verified by sequencing. The pGEX2TK-Nr2f2 protein plasmid was transformed into E. coli BL21 competent cells. Proteins were expressed and purified, and purified proteins were quantified against BSA. EMSAs were performed. Probes were generated by annealing 100 pmol of sense and antisense oligonucleotides, and 1-2 pmol of probe was used in each reaction. All primer and probe sequences are provided in Table 9. Gel shift reactions were conducted at 4° C. in 20% glycerol, 20 mM Tris (pH 8.0), 10 mM KCl, 1 mM DTT, 12.5 ng poly dI/C, 6.25 pmol of random, single-stranded oligonucleotides, BSA and the probe in the amount specified above. Samples involving the Nr2f2 protein were loaded on a 6% gel to resolve protein-DNA complexes. In reactions with cold competitors, 20x unlabeled probes were included in the reactions. Anti-NR2F2 antibody (R&D Biosystems™; cat #PP-H7147-00) was at the same amount of the Nr2f2 protein to obtain super-shifts.

The GEO accession number for the mammalian genomic data reported herein is GSE100910. The zebrafish genomic data reported herein was submitted to the NCBI™ Gene Expression Omnibus.

Real-time imaging can show blood circulation through region of ectopic niche endothelial gene expression. At 72 hpf show blood cells can be seen circulating through a region of vessels in the dorsal trunk that are ectopically expressing mrc1a:GFP in a 72 hpf embryo that had been injected with a pool of seven transcription factors at the one cell-stage.

Real-time imaging can show initial recruitment of HSPC to region of ectopic niche endothelial expression. A runx1⁺ HSPC can be seen initially lodging in a dorsal vessel that is ectopically expressing mrc1a:GFP (see e.g., FIG. 5D arrows) in a 72 hpf embryo that had been injected with a pool of ETV2, SOX7 and Nr2f2 at the one cell-stage. Black arrowhead points to HSPC localization in the CHT. The duration of the time-lapse is 6.5 hours; time intervals are 2 min. Time-series is shown in FIG. 5D.

Real-time imaging can show proliferation of HSPCs and egress from ectopic region of niche endothelial gene expression. Runx1⁺ HSPCs can be localized to a vessel ectopically expressing mrc1a:GFP (see e.g., FIG. 5E arrows) in a 72 hpf embryo that had been injected with a pool of ETV2, SOX7 and Nr2f2 at the one cell stage. HSPCs divide several times and migrate away into circulation. Black arrowhead points to HSPC localization in the CHT. The duration of the time-lapse is 2.6 hours; time intervals are 2 min. Time-series shown in FIG. 5E.

TABLE 1
CHT EC-enriched genes.

| | | | Associated | |
|---|---|---|---|---|
| | | CHT Expression | with CHT EC | |
| | | Confirmed by | ATAC-seq | |
| Gene | Full Gene Name | WISH | Element* | Function |
| adra1d | adrenoreceptor alpha 1D | No^{†} | Yes | G-protein coupled receptor; mitogenic |
| | | | | response activation |
| ap1b1 | adaptor-related protein complex 1, beta 1 | Yes | Yes | Coated vesicle clathrin recruitment |
| | subunit | | | |
| cldn11a | claudin 11a | Yes | Yes | Tight junction strand component |
| cltca | clathrin, heavy chain a (Hc) | Yes | Yes | Major coated vesicle and coated pit |
| | | | | component |
| ctsh | cathepsin H | Yes | No | Lysosomal cysteine proteinase |
| ctsla | cathepsin La | Yes | Yes | Lysosomal cysteine proteinase |
| dab2 | Dab, mitogen-responsive phosphoprotein, | n/a^{‡} | Yes | Mitogen-responsive phosphoprotein; |
| | homolog 2 (Drosophila) | | | clathrin-mediated endocytosis |
| exoc3l2a | exocyst complex component 3-like 2a | Yes | Yes | SNARE binding |
| glula | glutamate-ammonia ligase (glutamine | Yes | Yes | Glutamine synthesis |
| | synthase) a | | | |
| gpr182 | G protein-coupled receptor 182 | Yes | Yes | G-protein coupled receptor; vasodilation |
| hexb | hexosaminidase B (beta polypeptide) | Yes | Yes | Degradation of N-acetyl hexosamine |
| | | | | containing molecules |
| hyal2a | hyaluronidase 2a | n/a^{§} | Yes | Hyaluronan degradation |
| hyal2b | hyaluronidase 2b | Yes | Yes | Hyaluronan degradation |
| ifi30 | interferon, gamma-inducible protein 30 | Yes | Yes | Disulfide bond reduction. MHC class II- |
| | | | | restricted antigen processing |
| il13ra2 | interleukin 13 receptor, alpha 2 | Yes | No | Interleukin 13 binding |
| igmn | legumain | Yes | Yes | Hydrolysis of asparaginyl bonds |
| lyve1b | lymphatic vessel endothelial hyaluronic | Yes | Yes | Hyaluronan receptor |
| | receptor 1b | | | |
| man2b2 | mannosidase, alpha, class 2b, member 2 | Yes | Yes | Mannose glycosylase |
| mrc1a | mannose receptor, C type 1a | Yes | Yes | Glycoprotein endocytosis |
| npl | N-acetylneuraminate pyruvate lyase | n/a^{‡} | Yes | N-acetylneuraminic acid cleavage |
| | (dihydrodipicolinate synthase) | | | |
| prcp | prolylcarboxypeptidase (angiotensinase C) | n/a^{‡} | Yes | C-terminal proline linked amino acid |
| | | | | cleavage |
| pxk | PX domain containing serine/ | No | Yes | Synaptic transmission |
| | threonine kinase | | | |
| sele | selectin E | Yes | Yes | Endothelial cell |
| | | | | adhesion to blood leukocytes |
| sepp1a | selenoprotein P | Yes | No | Selenium binding |
| slc16a9a | solute carrier family 16, member 9a | No | Yes | Symporter activity |
| snx8a | sorting nexin 8a | Yes | Yes | Phosphatidylinositol binding |
| stab1 | stabilin 1 | Yes | Yes | Scavenger receptor activity |
| stab2 | stabilin 2 | Yes | Yes | Scavenger receptor activity, hyaluronan |
| | | | | receptor |
| til1 | tolioid-like 1 | Yes | Yes | Procollagen C-propeptide processing |

TABLE 2
In vivo screening of predicted enhancer elements.

| | | | | | Showed Predicted | Element Contains |
|---|---|---|---|---|---|---|
| Type of | Gene | Genomic Coordinates of | Relative to | Amplicon | GFP Expression | Ets, SoxF and |
| Element | Name | ATAC-seq Element* | TSS (kb) | Size (bp) | Pattern^{†} | NHR Motifs |
| CHT EC | ap1b1 | chr 5:26463217-26463695 | 17 | 750 | Yes | Yes |
| Element | cltca | chr10:29,047,274-29,047,619 | 2.8 | 404 | Yes | Yes |
| | dab2 | chr5:33,980,000-33,980.306 | −3.5 | 394 | Yes | Yes |
| | exoc3l2a | chr5:38359097-38359903 | 5.9 | 901 | Yes | Yes |
| | glula | chr2:19,458,704-19,459.047 | 4.8 | 446 | No | Yes |
| | gpr182 | chr23:36701205-36701682 | −4.9 | 481 | Yes | Yes |
| | gpr182 | chr23:36694073-36694476 | −2.8 | 398 | Yes | Yes |
| | gpr182 | chr23:36696363-36696656 | 1.6 | 577 | Yes | Yes |
| | lgmn | chr13:36,448,465-36,448,818 | 2.9 | 414 | Yes | Yes |
| | prcp | chr15:10,400,588-10,400,868 | 23 | 334 | No | No^{‡} |
| | sele | chr20:34,010,027-34,010,326 | −9.7 | 398 | Yes | Yes |
| | sele | chr20:34,011,251-34,011,563 | −8.5 | 360 | Yes | Yes |
| | snx8a | chr3:42,090,805-42,091,062 | 5.5 | 395 | No | Yes |
| | stab1 | chr22:10467346-10467937 | −2.8 | 874 | Yes | Yes |
| | stab2 | chr4:9790795-9791116 | 4.3 | 422 | Yes | Yes |
| Pan-EC | cdh5 | chr7:45457842-45458791 | 13 | 823 | Yes | Yes |
| Element | clec14a | chr17:10362325-10362844 | −3.1 | 455 | Yes | Yes |
| | dll4 | chr20:28219013-28219619 | −55 | 452 | Yes | No^{‡} |
| | fli1a | chr18:47039842-47040466 | 47 | 800 | Yes | No^{‡} |
| | lmo2 | chr18:36722030-36722527 | −3.6 | 367 | Yes | No^{‡} |
| | nrp1b | chr2:43535098-43535801 | −34 | 552 | Yes | Yes |

Table 1 shows CHT EC genes identified by tomo-seq and tissue-specific RNA-seq. Asterisks (*) indicates that gene is within 100 kb of TSS; some genes are associated with multiple elements. Cross (†) indicates that no expression was observed by WISH. Double cross (‡) indicates that WISH was not attempted but CHT expression was reported on the world web wide available at zfin.org. Double S (§) indicates that WISH was not attempted.

Table 2 shows CHT EC-specific and pan-EC ATAC-seq elements that were fused to a minimal promoter and GFP and injected into one-cell stage zebrafish embryos. Asterisks (*) indicates coordinates of MACS2 peak. Cross (†) indicates expression in CHT ECs for CHT EC elements and in vessels throughout the embryo for pan-EC elements. Double cross (‡) indicates lack of NHR motif.

TABLE 3
CHT-enriched genes identified by tomo-seq

| | | CHT Expression |
|---|---|---|
| Gene | Full Gene Name | Confirmed by WISH |
| abi3bp | ABI family, member 3 (NESH) binding protein | Yes |
| ACKR3 | atypical chemokine receptor 3 | No* |
| adam8a | ADAM metallopeptidase domain 8a | Yes |
| adra1d | adrenoceptor alpha 1D | No* |
| adr53b | adrenoceptor beta 3b | No* |
| agrP | agouti related neuropeptide | No* |
| ANGPT4 | angiopoietin 4 | No* |
| ap1b1 | adaptor-related protein complex 1, beta 1 subunit | Yes |
| apinra | apelin receptor a | Yes |
| agp7 | aguaporin 7 | Yes |
| atp1a1a.2 | ATPase Na+/K+ transporting subunit alpha la, | n/a^{†} |
| | tandem duplicate 2 | |
| atp1b1b | ATPase, Na+/K+ transporting, beta 1b | n/a^{†} |
| ba1 | ba1 globin | n/a^{†} |
| blf | bloody fingers | n/a^{†} |
| BX005069.4 | leukocyte cell-derived chernotaxin-2-like | n/a^{†} |
| BX323861.1 | SLAM family member 9-like isoform X2 | n/a^{†} |
| C10H8orf4 | | n/a^{†} |
| ca15a | carbonic anhydrase XVa | n/a^{†} |
| CABZ01049362.1 | PREDICTED: GTPase IMAP family member 4-like | n/a^{†} |
| | [Danio rerio]. or 8-like | n/a^{†} |
| CABZ01058863.1 | | n/a^{†} |
| CABZ01066695.1 | PREDICTED: protein lyl-1-like isoform X1 | n/a^{†} |
| | [Danio renio]./.lymphocytic leukemia protein | |
| ccdc88b | coiled-coil domain containing 88B | n/a^{†} |
| ccr9a | chemokine (C-C motif) receptor 9a | Yes |
| CD209 | CD209 molecule | n/a^{†} |
| cd28 | CD28 molecule | Yes |
| ceacam1 | carcinoembryonic antigen-related cell adhesion | Yes |
| | molecule 1 | |
| ch25hl2 | cholesterol 25-hydroxylase like 2 | n/a^{†} |
| cldn11a | claudin 11a | Yes |
| cfdng | claudin g | n/a^{†} |
| cltca | clathrin, heavy chain a (Hc) | Yes |
| cmklr1 | chemokine-like receptor 1 | n/a^{†} |
| cndp2 | camosine dipeptidase 2 | Yes |
| cnn1a | calponin 1, basic, smooth muscle, a | Yes |
| COL19A1 | collagen, type XIX, alpha 1 | Yes |
| col28a1 | collagen, type 28, alpha 1 | n/a^{†} |
| coro1a | coronin, actin binding protein, 1A | n/a^{†} |
| cpa5 | carboxypeptidase A5 | n/a^{†} |
| CR381673.2 | Natural killer cell receptor 2B4-like isorm x1/2 or | n/a^{†} |
| | SLAM family member7/9- | |
| ctsh | cathepsin H | Yes |
| ctsla | cathepsin La | Yes |
| CU463790.1 | | Yes |
| CU861664.1 | PREDICTED: zinc finger protein 521-like [Danio rerio] | n/a^{†} |
| CU915778.1 | CU915778.1 | No* |
| cyp24a1 | cytochrome P450, family 24, subfamily A, | No* |
| | polypeptide 1 | |
| cysltr1 | cysteinyl leukotriene receptor 1 | Yes |
| dab2 | Dab, mitogen-responsive phosphoprotein, homolog 2 | n/a^{†} |
| | (Drosophila) | |
| drl | draculin | n/a^{†} |
| ela2 | elastase 2 | Yes |
| ENSDARG00000075833 | lymphatic vessel endothelial hyaluronic receptor 1a/b | Yes |
| entpd2a.1 | ectonucleoside triphosphate diphosphohydrolase 2a, | n/a^{†} |
| | tandem duplicate 1 | |
| exoc3l2a | exocyst complex component 3-like 2a | Yes |
| f2r | coagulation factor II (thrombin) receptor | Yes |
| foxi3b | forkhead box l3b | n/a^{†} |
| frem1b | Fras1 related extracellular matrix 1b | Yes |
| gcm2 | glial cells missing homolog 2 (Drosophila) | Yes |
| glud1a | glutamate dehydrogenase 1a | Yes |
| glula | glutamate-ammonia ligase (glutamine synthase) a | Yes |
| GMIP | GEM interacting protein | n/a^{†} |
| gpr182 | G protein-coupled receptor 182 | Yes |
| grap2b | GR82-related adaptor protein 2b | n/a^{†} |
| gsto1 | glutathione S-transferase omega 1 | Yes |
| havcr1 | hepatitis A virus cellular receptor 1 | n/a^{†} |
| hbaa1 | hemoglobin, alpha adult 1 | n/a^{†} |
| hdr | hematopoietic death receptor | n/a^{†} |
| hexb | hexosaminidase B (beta polypeptide) | Yes |
| hyal2a | hyaluronidase 2 | n/a^{†} |
| hyal2b | hyaluronidase 2 | Yes |
| HYAL2 | hyaluronidase 2 | Yes |
| ifi30 | interferon, gamma-inducible protein 30 | Yes |
| il10ra | interleukin 10 receptor, alpha | n/a^{†} |
| il13ra2 | interleukin 13 receptor, alpha 2 | Yes |
| il6r | interleukin 6 receptor | Yes |
| ITGAE | integrin, alpha E, tandem duplicate 1/2 | No* |
| itgb2 | integrin, beta 2 | Yes |
| kcnj1a.3 | potassium inwardly-rectifying channel, subfamily J, | n/a^{†} |
| | member 1a, tandem duplicate 3 | |
| kcnj1a.5 | potassium inwardly-rectifying channel, subfamily J, | n/a^{†} |
| | member 1a, tandem duplicate 5 | |
| lamp2 | lysosomal-associated membrane protein 2 | Yes |
| lgals9l1 | lectin, galactoside-binding, soluble, 9 (galectin 9)-like 1 | n/a^{†} |
| lgmn | legumain | Yes |
| lpar5a | lysophosphatidic acid receptor 5a | n/a^{†} |
| mafbb | v-maf avian musculoaponeurotic fibrosarcoma | n/a^{†} |
| | oncogene homolog Bb | |
| man2b2 | mannosidase alpha, class 2B, member 2 | Yes |
| marco | macrophage receptor with collagenous structure | n/a^{†} |
| MCOLN2 | mucolipin 2[WM2] | n/a^{†} |
| mir142a | micro RNA 142a | n/a^{†} |
| mmp13a | matrix metallopeptidase 13a | n/a^{†} |
| MOV10L1 | putative helicase Mov10l1 [Danio rerio]. 95% ident. | n/a^{†} |
| mpx | myeloid-specific peroxidase | n/a^{†} |
| mrc1a | mannose receptor, C type 1a | Yes |
| mrc1b | mannose receptor, C type 1b | n/a^{†} |
| myh11a | myosin, heavy chain 11a, smooth muscle | Yes |
| myha | myosin, heavy chain a | n/a^{†} |
| myo1f | myosin IF | Yes |
| ncf1 | neutrophil cytosolic factor 1 | n/a^{†} |
| npl | N-acetylneuraminate pyruvate lyase | n/a^{†} |
| | (dihydrodipicolinate synthase) | |
| ostf1 | osteoclast stimulating factor 1 | n/a^{†} |
| parvg | parvin, gamma | Yes |
| pdia2 | protein disulfide isomerase family A, member 2 | n/a^{†} |
| PLCXD1 | phosphatidylinositol-specific phospholipase C, | n/a^{†} |
| | X domain containing 1 | |
| plek | pleckstrin | n/a^{†} |
| polm | polymerase (DNA directed), mu | n/a^{†} |
| prcp | prolylcarboxypeptidase (angiotensinase C) | n/a^{†} |
| pxk | PX domain containing serine/threonine kinase | No* |
| rasal3 | RAS protein activator like 3 | n/a^{†} |
| RNF223 | ring finger protein 223 | n/a^{†} |
| s1pr4 | sphingosine-1-phosphate receptor 4 | n/a^{†} |
| sele | selectin E | Yes |
| sepp1a | selenoprotein P | Yes |
| setx | senataxin | Yes |
| si:ch1073-429i10.1 | si:ch1073-429i10.1 | Yes |
| si:ch211-214p16.1 | si:ch211-214p16.1 | No* |
| si:ch211-214p16.2 | si:ch211-214p16.2 | Yes |
| si:ch211-250g4.3 | PREDICTED: nesprin-1 isoform X4 [Danio rerio] | n/a^{†} |
| si:ch211-284o19.8 | si:ch211-284o19.8 | n/a^{†} |
| si:ch211-285f17.1 | si:ch211-285f17.1 | n/a^{†} |
| si:ch211-67e16.2 | cd28-like molecule | n/a^{†} |
| si:ch73-248e21.7 | si:ch73-248e21.7 | n/a^{†} |
| si:ch73-27e22.6 | si:ch73-27e22.6 | n/a^{†} |
| si:dkey-102g19.3 | si:dkey-102g19.3 | n/a^{†} |
| si:dkey-188i13.7 | interferon alpha inducible protein 46 | n/a^{†} |
| si:dkey-237j10.2 | si:dkey-237j10.2 | n/a^{†} |
| si.dkey-33i11.4 | si:dkey-33i11.4 | Yes |
| si:dkey-69c1.1 | si:dkey-69c1.1 | n/a^{†} |
| skap2 | src kinase associated phosphoprotein 2 | n/a^{†} |
| sla1 | src-like-adaptor 1 | n/a^{†} |
| slc16a9a | solute carrier family 16, member 9a | No* |
| slc4a11 | solute carrier family 4, sodium borate transporter, | n/a^{†} |
| | member 11 | |
| snx8a | sorting nexin 8a | Yes |
| srgn | serglycin | Yes |
| stab1 | stabilin 1 | Yes |
| stab2 | stabilin 2 | Yes |
| syk | spleen tyrosine kinase | n/a^{†} |
| tagapb | T-cell activation RhoGTPase activating protein b | n/a^{†} |
| til1 | tolloid-like 1 | Yes |
| tmem106a | transmembrane protein 106a | Yes |
| tnfsf12 | TNF superfamily member 12 | n/a^{†} |
| tnni1b | troponin I type 1b (skeletal, slow) | Yes |
| tubb1 | tubulin, beta 1 class VI | n/a^{†} |
| wasa | Wiskott-Aldrich syndrome (eczema-thrombocytopenia) a | n/a^{†} |
| wasb | Wiskott-Aldrich syndrome (eczema-thrombocylopenia) b | n/a^{†} |
| WIPF1 | WAS/WASL interacting protein family, member 1a/b | n/a^{†} |
| zgc:158446 | complement factor b, like | n/a^{†} |
| zgc:174945 | zgc:174945 | n/a^{†} |
| zgc:198419 | fenitin, heavy polypeptide-like 28 | Yes |

Asterisk (*) in Table 3 indicates that no CHT expression was observed by WISH.
Dagger symbol (†) in Table 3 indicates WISH was not attempted.

TABLE 4
Transcription factor expression in CHT ECs

| | | | Associated with CHT | |
|---|---|---|---|---|
| | | | EC ATAC-seq | |
| Transcription | | | Element Containing Ets, | Genomic Coordinates |
| Factor | Family | FPKM | Sox and NHR Sites* | of Representative Element |
| fli1a | Ets | 480.4 | Yes | chr18:46966409-46966698 |
| etv2 | Ets | 192.3 | Yes | chr16:44782409-44782895 |
| ets1 | Ets | 183 | Yes | chr18:46883643-46884100 |
| sox18 | SoxF | 206.4 | Yes | chr23:8886011-8886744 |
| sox7 | SoxF | 125.1 | Yes | chr20:19158376-19158663 |
| nr2f2 | NHR | 84.6 | Yes | chr18:23728906-23729747 |
| rxraa | NHR | 45.9 | Yes | chr21:16411020-16411531 |

Table 4 shows FPHM expression values in CHT ECS for highly expressed members of the Ets, Sox and NHR transcription factor families. Asterisk (*) in Table 4 indicates within 100 kb of TSS; some genes are associated with multiple elements.

TABLE 5
Transcription factor expression in mouse hematopoetic niche

| Transcription | | Mouse E14-E15 Liver | Mouse E16-E17 Liver | Mouse Adult Bone Marrow |
|---|---|---|---|---|
| Factor | Family | EC FPKM | EC FPKM | EC FPKM |
| Ets1 | Ets | 218.4666 | 251.9493 | 153.2657 |
| Erg | Ets | 46.64156 | 78.53131 | 45.14673 |
| Elk4 | Ets | 9.369453 | 11.4226 | 22.83457 |
| Elk1 | Ets | 7.003965 | 9.08418 | 6.8779 |
| Etv1 | Ets | 2.203135 | 3.10327 | 1.488542 |
| Etv2 | Ets | 0.235977 | 0 | 0 |
| Sox18 | SoxF | 127.1509 | 262.44 | 130.1783 |
| Sox7 | SoxF | 49.94503 | 46.9365 | 19.80563 |
| Sox17 | SoxF | 33.01219 | 68.37438 | 90.24645 |
| Sox11 | SoxF | 12.01665 | 11.1584 | 0.67509 |
| Sox12 | SoxF | 11.81507 | 21.5267 | 0.556478 |
| Sox6 | SoxF | 1.741399 | 1.158524 | 0.51182 |
| Sox5 | SoxF | 0.193041 | 0.289841 | 0.437005 |
| Sox9 | SoxF | 0.119527 | 0.072563 | 0 |
| Nr2f2 | NHR | 58.97832 | 103.5558 | 63.17458 |
| Rxra | NHR | 23.98264 | 33.0942 | 22.08392 |
| Rara | NHR | 19.29841 | 27.37294 | 13.93433 |
| Nr4a2 | NHR | 10.13413 | 3.130986 | 30.30394 |
| Esrrb | NHR | 6.219864 | 7.884516 | 0.586381 |
| Rora | NHR | 1.219604 | 1.086872 | 5.922048 |

TABLE 6
Primers used for WISH probe synthesis (“Forward” primers disclosed
as SEQ ID NOS 16-84 and “Reverse” primers disclosed as SEQ ID NOS
85-153, all respectively, in order of appearance)

| Category | Gene | Forward* | Reverse^{†} |
|---|---|---|---|
| CHT EC enriched | adra1d | GCTCCATAGTATCGTCTGAACC | AAACCATTGCCATTTTGCCA |
| CHT EC enriched | ap1b1 | GGGAGTTCTTCGGGTGACTG | GCTTGCAACAAAAAGCGCAG |
| CHT EC enriched | cldn11a | TGTGTGATCTCAACTGCGCT | GGTGCAATCTAGTCTGATCGGT |
| CHT EC enriched | cltca | CCAGCAAACCCCATGGATCT | AACCGAGTACAGGACACACG |
| CHT EC enriched | ctsh | CGACTGGAGAACCAAGGGAC | TGGAGGCTAATCGAGTGTGC |
| CHT EC enriched | ctsla | CCATGCAACAGAGGAAGGGT | TACTGGGCGGGTCTCCTTTA |
| CHT EC enriched | exoc3l2a | AAGTTCCGCAGGATGGACTG | TCGCTTGTGTGATCAAGTATGAC |
| CHT EC enriched | glula | AGTTATGCCAGCTCAGTGGG | GGCCTCCCCAAGAAACCATT |
| CHT EC enriched | gpr182 | CTTCCCACAGCAGCACAAAC | GAAAGTTGTTGTTGAAGTGAACG |
| CHT EC enriched | hexb | GAATTTGCTCGCATGAGGGG | CGGCAGTGGCCAACAAATAG |
| CHT EC enriched | hyal2b | ATGGAGGTCTACCACAGGCT | AGTGCAGGTATGTGTCCGTG |
| CHT EC enriched | ifi30 | TTCGGCTTTAACCTGTGCGT | CCTGACGCGAGTAGTGTTGT |
| CHT EC enriched | il13ra2 | AGTTAGAATGGGCGCCACC | GGCAAGACCACTGGCATTTG |
| CHT EC enriched | lgmn | AACTTGAGCCACCGAGGATTT | CCCTAACTCCAGCACACACT |
| CHT EC enriched | lyve1b | GCTACAGTCTGCGTAGCAT | TGGAAGCAGCTCTAAGTGACAG |
| CHT EC enriched | man2b2 | TACCCAATGGTTCGAGTGGC | GCTTAGGTGATCAATTTTGGGACA |
| CHT EC enriched | mrc1a | GTGTCCCCTCATCAATGCCA | ACGGCATTCCACAAACCAGA |
| CHT EC enriched | sele | TGCCCAGCCCTTGATAATCT | ACCCAAACTGACTTTATATGTGC |
| CHT EC enriched | sepp1a | AGGCAGCACTGGACTTTAGC | AGGTACAAATGCAAGTACAACACT |
| CHT EC enriched | snx8a | ACAAAGAGATCTGCATTCCAAGC | AGCCTGTCAGCTCACTTTATT |
| CHT EC enriched | stab1 | AAGGCGTACTATGTCCTCAGGC | CGCCGTTCTATAATGCACCG |
| CHT EC enriched | stab2 | TTGTGGATTACGGGGTTCGG | AAAGAGAGCTGCACCGACT |
| CHT EC enriched | tll1 | GAGCTTTACTCTGCTGGCGA | ACAAATGATGTCTGTCTCCGCT |
| CHT enriched | abi3bp | CTGTTTTCCCCCACCAGTGA | CAAAGGATTGGCAGGGACCA |
| CHT enriched | ackr3 | TGGGATTTATTTGTAACTACACGGA | TTTTAAGCACATTTCTGAAGCACA |
| CHT enriched | adam8a | CCAGGAAGCGCAAAGAACAG | ACATTAGGCGGGCAAAACAAA |
| CHT enriched | adrb3b | GCAGCAAACGACTGCTACAA | CCCACTTCGCTGCTCTTTAC |
| CHT enriched | agrp | TCATCCACACCTGAGACGCA | ACACCTTAAAACCGCAGCC |
| CHT enriched | angpt4 | ATCCGACTGCTGGAATGGAC | GCTTTGAGGAGCTTAAGAGGC |
| CHT enriched | aplnra | GTGCTGGTCAACATGTACGC | CGTCACTTTTCACCCCCAGA |
| CHT enriched | aqp7 | TCCACTGGGAAAAGCTGGAAT | TTTCAGATGCAGCACAGGCA |
| CHT enriched | bnip3lb | ATGGGGCTGACGGATACC | GCACAGGAAACGCACATGAT |
| CHT enriched | ccr9a | TTGTCCAGACTACCAAGGCG | TTACTTCACTGCCAGTCGGC |
| CHT enriched | cd28 | ATCCAAACTGAGGCCGGAAG | AGAAAATACAGTGCATACATGTCAA |
| CHT enriched | ceacam1 | GGCCCAAGCATGGCAGAAAC | CCTACAAGCCTCATTCAGACAGT |
| CHT enriched | cndp2 | ACATGGGACATGGAGCGAAG | ACACTAGAAAACCGATCGTGTCA |
| CHT enriched | cnn1a | GACTCTCTGCGGATGTCAGG | GGTCATGCCCTTTTGGCTTG |
| CHT enriched | col19a1 | CATGTCCACCCCTGAAGCTG | GGGTTCTGTTGTGGAGTGCT |
| CHT enriched | cu463790.1 | GGCGTCTCTTTTTCTGCTGC | TGACGCTTAAACAGAGCGGT |
| CHT enriched | cu915778.1 | CCCTAGTGTCCGAGGTCTCA | TTTCCCCTGTGTGGATGAGC |
| CHT enriched | cyp24a1 | GATACCGTGCTGGGCGATTA | CCACCACTCACTCATTCAGACA |
| CHT enriched | cysltr1 | TCCCGGTGCAAAATCTGAGG | AGTCATGCACAAAATCTGCGG |
| CHT enriched | ela2 | GTTTATTGCTGGCGCCTACG | TTCTTGGGGTAGTTGCAGCC |
| CHT enriched | f2r | GCTGCCGAACAACGAAACAT | TAGGACGCGTCATTGTGCTT |
| CHT enriched | frem1b | AGTACACTCCGGACCCAAGA | CACCAGAAAGAATGTCACCGT |
| CHT enriched | gcm2 | TCCAGAGCGATTCAGCATCA | CAGTCCCTCAGTATTCCCCG |
| CHT enriched | glud1a | AGTCTCCTACTTCGAGTGGCT | ACGCCTGAGATTCATCCTGC |
| CHT enriched | ll6r | AACTGTTCTTTCTCCCGGTCCC | CCTCTGGCTGAACAGGAAGG |
| CHT enriched | itgae | ACTGGTCAACCACCTCCTCT | ACACAATCAGGCAAGGTCTC |
| CHT enriched | itgb2 | TGCCTTTCAAAGTGGACCGT | ACCAGTCACACCAGCCATTC |
| CHT enriched | lamp2 | AGCCTGTTCCTGGACCATTG | AGCTACAACCATTGAGGGCT |
| CHT enriched | myh11a | GGTTCGCCAGAAGGACAAGA | AGCATCCAAAAGTACTCGGTG |
| CHT enriched | myo1f | AAGCTGTCATCAAAGCCGGA | TTCTCGACCTGTCAGCTGTT |
| CHT enriched | parvg | TGAAAGCCCTGAACGAGACC | CGTCAGCATCCAAACGCAAT |
| CHT enriched | setx | AGGAGTTTGGCTTCGACCAG | GTGACGCTGGAATATCCCGT |
| CHT enriched | skch1073-429l10.1 | TCGCTCTGATGCTCAGCTTG | CACTCGGCGACAGTATTCCC |
| CHT enriched | skch211-214p16.1 | TACACATTTTCTGCCCCACTGA | AATGGGGCAAGAGTCCATCT |
| CHT enriched | skch211-214p16.2 | CTCACCCTCGGTCCAGAACT | ACAGACACACTTGCCAGTCA |
| CHT enriched | skdkey-33i11.4 | ACAGCCATCAGTTCCTCTGC | AGCTTTGCATCCCCATCACT |
| CHT enriched | srgn | GGAAGCCACTCCTGATACGG | GTACAACATTTACTTGCTGTCCA |
| CHT enriched | tmem106a | GGTCACCGCACCAAATGAACC | AACAGTTCTGATTGGATTTTGCTCA |
| CHT enriched | tnni1b | TCTGCATCTCGCAAGCTGAT | CATGTGTAGTGCAGACAGAACA |
| CHT enriched | zgc: 198419 | AGAACTACGACAGCGACTGC | GGTTTTGGATAAGAGCTGTGTCA |
| Transcription | ets1 | ACAGACTCTGTACGTTTGAATGCGT | GTCCAGACTTTACTCGTCCGTGTC |
| Factor | | | |
| Transcription | etv2 | TATGACTGCAGTGGTGAAGACC | CTTTCCCGCCGTTTTGTGAA |
| Factor | | | |
| Transcription | fli1a | CAGACCCGTCTCTGTGGTC | CCAGTATGGGGTTGTGGGAC |
| Factor | | | |
| Transcription | nr2f2 | ACCCCCGAACAACAATAACA | AGAGGGCAAGCGCAGTAATA |
| Factor | | | |
| Transcription | sox7 | TATAGCCCTTCGTTCCCCCA | ACCGAAACCGGCTAAACTGA |
| Factor | | | |
| Transcription | sox18 | TCCTTGGACGCTGTGGACCAAC | TCAAAGCGCTGCTTTCCTCGC |
| Factor | | | |

Asterisk (*) in Table 6 indicates that the T3 sequence CATTAACCCTCATAAAGGGAA (SEQ ID NO: 10) was
added to the 5′ end of each forward primer. Dagger symbol (†) in Table 6 indicates that The T7
sequence TAATACGACTCACTATAGGG (SEQ ID NO: 11) was added to the 5′ end of each reverse primer.

Asterisk (*) in Table 6 indicates that the T3 sequence CATTAACCCTCATAAAGGGAA (SEQ ID NO: 10) was added to the 5′ end of each forward primer. Dagger symbol (†) in Table 6 indicates that The T7 sequence TAATACGACTCACTATAGGG (SEQ ID NO: 11) was added to the 5′ end of each reverse primer.

TABLE 7
Primers used to clone promoter and enhancer elements (“Forward”
primers disclosed as SEQ ID NOS 154-178 and “Reverse” primers disclosed
as SEQ ID NOS 179-203, all respectively, in order of appearance)

| | | | Amplicon | | |
|---|---|---|---|---|---|
| Type of | | Genomic Coordinates | Size | | |
| Element | Gene | of ATAC-seq Element | (bp) | Forward | Reverse |
| 5′ upstream | mrc1a | chr7: | 1353 | CTTTTGGCCATTACTGCCG | TTCTGTCTTTTAATCAGCAATCC |
| of TSS | (1.3 kb) | 65,468,213-65,469,565 | | | |
| CHT EC | mrc1a | chr7: | 125 | GCTCTCAGTTCCTGGTATT | TGAAGCTTGTACCTTTCATTTCC |
| element | (125 bp) | 65469086-65469210 | | TTTCT | |
| 5′ upstream | sele | chr20: | 5301 | TCGTTACTGCACTTGAAAG | TATCAGTGATGTTCTGCAGTGGT |
| of TSS | (5.3 kb) | 34,001,481-34,006,781 | | CGT | C |
| CHT EC | sele | chr20: | 158 | CCATGAAACTGGGAAGATG | CAGGAAGAAATAATGGCAAAAA |
| element | (158 bp) | 24004805-34004962 | | AA | |
| CHT EC | ap1b1 | chr5: | 750 | GAAGCTCTCCAGCAGCTCA | CATTTCCACCAGCTGTCTGAT |
| element | | 26463217-26463695 | | | |
| CHT EC | cltca | chr10: | 404 | GCTGTCAGCACATTCTTTT | CCCTGCTGATCACACATGAC |
| element | | 29,047,274-29,047,619 | | CC | |
| CHT EC | dab2 | chr5: | 394 | ACTGCTCCTCACCAATCGT | TGCACTAAATCTGTGCCAAGTC |
| element | | 33,960,000-33,980,306 | | C | |
| CHT EC | exoc3l2a | chr5: | 901 | TTTATATAATCGGAAGGAA | TCCTGTCAGCTGTTTTCATCC |
| element | | 38359097-38359903 | | CCTTTTT | |
| CHT EC | glula | chr2: | 446 | GGCAAAATGCTTAGATGCA | TGCGAGGAGGACATAACAA |
| element | | 19,458,704-19,459,047 | | GA | |
| CHT EC | gpr182 | chr23: | 481 | TAGCCTTGTGCAATGCTTG | TGCTGAATTCAAAAGCCACTT |
| element | | 36701205-36701682 | | T | |
| CHT EC | gprf82 | chr23: | 398 | CACTTCTGGTACCAAATGA | GAGGGTTAAACGTGGCCTTA |
| element | | 36694073-36694476 | | TCAAC | |
| CHT EC | gprf182 | chr23: | 577 | GCGGCAAACTTTTTGAGTG | GCCAGCCTCAAAGTTTGTTCT |
| element | | 36696363-36696656 | | T | |
| CHT EC | lgmn | chr13: | 414 | CGCGTGATGAGGATCTGAT | GGTGTTGAAAGGTGATGCTG |
| element | | 36,448,465-36,448,818 | | T | |
| CHT EC | prcp | chr15: | 334 | AAAATTAAGAGCGGGCAGA | TGGAAACAACAACAGCCTGA |
| element | | 10,400,588-10,400,868 | | CT | |
| CHT EC | sele | chr20: | 398 | AAAGCACTTGATTGAGAAT | TGTTTGGTTCAGTTACACGTTTT |
| element | | 34,010,027-34,010,326 | | TGC | |
| CHT EC | sele | chr20: | 360 | CAGTTTCCCAAGCTTCAAG | TGTGATTACACATTCCCACACAT |
| element | | 34,011,251-34,011,563 | | G | |
| CHT EC | snx8a | chr3: | 395 | AATGGTTGCAGCATTGTGT | GCTTTTGTTTGGTGATGTGC |
| element | | 42,090,805-42,091,062 | | T | |
| CHT EC | stab1 | chr22: | 874 | GTTACCTGGCAACCACCAA | TGGTCAGAATAAGCACGTTTCA |
| element | | 10467346-10467937 | | C | |
| CHT EC | stab2 | chr4: | 422 | ACGTTAACAAGGCGATGTT | TCTAAACAATTTTTAAGGTAAAC |
| element | | 9790795-9791116 | | TT | CAAA |
| Pan-EC | cdh5 | chr7: | 823 | TGACAGGACTCATCAGCAC | AATAGTCTCTGGTCTGCTGTTAAA |
| element | | 45457842-45458791 | | G | |
| Pan-EC | clec14a | chr17: | 455 | TGGGAAAAATACCAGGAAG | AAGCAGCGAGCTCTCATAATAAA |
| element | | 10362325-10362844 | | CGT | |
| Pan-EC | dll4 | chr20: | 452 | AGATCAATGAGAGCGAGGC | GGAGCAGATGAGGTTAAGTCCT |
| element | | 28219013-28219619 | | G | |
| Pan-EC | fli1a | chr18: | 800 | CGGACAGTAATGTCTGGAT | CCACAACTCCATACTGGGAAA |
| element | | 47039842-47040466 | | GG | |
| Pan-EC | lmo2 | chr18: | 367 | TCATCATGGCCAACAGAAT | GTGCAGGAAATGAGCACAGA |
| element | | 36722030-36722527 | | G | |
| Pan-EC | nrp1b | chr2: | 552 | TGACTCAACCAATCAATCA | TAGCAAAGCTCTCAGGCCC |
| element | | 43535098-43535801 | | GCCT | |

TABLE 8
Sequences and primers for mutational variants of the 125 bp mrc1a
and 158 bp sele enhancer elements (“Total Fragment Sequences”
disclosed as SEQ ID NOS 204-208, 13 and 209-212, “Forward Primers”
disclosed as SEQ ID NOS 213-222 and “Reverse Primers” disclosed as
SEQ ID NOS 223-232, respectively, in order of appearance)

| | Fragment | | | |
|---|---|---|---|---|
| Gene | Name | Total Fragment Sequence | Forward Primer | Reverse Primer |
| mrc1a | Wild-type | CCATGAAACTGGGAAGATGAAAGC | TGAAGCTTGTACCTTTCATTTCCTTT | GCTCTCAGTTCCTGGTATTTTTCTTTC |
| | | ATAGTTGAATTGTTACTGGCAACA | TTGCTGAGCTTTATTTTCTCTAGAAT | AGCTGAAAAAAAAATGCTGATTTGCTA |
| | | TCTTCTCTGTAATGCCCCCTGTGA | TGCCATTGTGTTTCCATTCTAG | GAATGGAAACACAATGGCAAT |
| | | CCCATATTGTCTCGCTCTTTCCTT | | |
| | | TATAAACAGAGCTGTAGATATCCA | | |
| | | CAGGAAATGGGGGTGTTTTTGCCA | | |
| | | TTATTTCTTCCTGTGAAGCTTGTA | | |
| | | CCTTTCATTTaaaTTTTG | | |
| mrc1a | Ets | CTGAGCTTTATTTTCTCTAGAATT | TGAAGCTTGTACCTTTCATTTaaaTT | GCTCTCAGTaaaTGGTATTTTTCTTTC |
| | mutant | GCCATTGTGTTTCCATTCTAGCAA | TTGCTGAGCTTTATTTTCTCTAGAAT | AGCTGAAAAAAAAATGCTGATTTGCTA |
| | | ATCAGCATTTTTTTTTCAGCTGAA | TGCCATTGTGTTTCCATTCTAG | GAATGGAAACACAATGGCAAT |
| | | AGAAAAATACCAtttACTGAGAGC | | |
| | | TGAAGCTTGTACCTTTCATTTCCT | | |
| | | TTTTG | | |
| mrc1a | Sox | CTGACGgggcggggaTCTAGAATT | TGAAGCTTGTACCTTTCATTTCCTTT | GCTCTCAGTTCCTGGTATTTTTCTTTC |
| | mutant | GCacggtgGTTTCCATTCTAGCAA | TTGCTGAGCgggcggggaTCTAGAAT | AGCTGAAAccccccgGCTGATTTGCTA |
| | | ATCAGCcggggggTTTCAGCTGAA | TGCacggtgGTTTCCATTCTAG | GAATGGAAACcaccgtGCAAT |
| | | AGAAAAATACCAGGAACTGAGAGC | | |
| | | attAGCagatTtaaTTTCATTTCC | | |
| | | TTTTTGCa | | |
| mrc1a | NHR | ttaaTTTATTTTCTCTAGAATTGC | attAGCagatTtaaTTTCATTTCCTT | GCTCTCAGTTCCTGGTATTTTTCTTTC |
| | mutant | CATTGTGTTTCCATTCTAGCAAAT | TTTGCattaaTTTATTTTCTCTAGAA | AGCTGAAAAAAAAATGCTGATTTGCTA |
| | | CAGCATTTTTTTTTCAGCTGAAAG | TTGCCATTGTGTTTCCATTCTA | GAATGGAAACACAATGGCAAT |
| | | AAAAATACCAGGAACTGAGAGCTG | | |
| | | AAGCTTGTACCTTTCATTTCCTTT | | |
| | | TTG | | |
| mrc1a | Control | CTGAGCTTTATTTTCTCTAGAATT | TGAAGCTTGTACCTTTCATTTCCTTT | GCTCTCAGTTCCTGGTATTTTTCggTC |
| | mutant | GCCATTGTGTTTCCATTCcgGCAA | TTGCTGAGCTTTATTTTCTCTAGAAT | AGCTGAAAAAAAAATGCTGATTTGCcg |
| | | ATCAGCATTTTTTTTTCAGCTGAc | TGCCATTGTGTTTCCATTCTAG | GAATGGAAACACAATGGCAAT |
| | | cGAAAAATACCAGGAACTGAGAGC | | |
| | | CCATGAAACTGGGAAGATGAAAGC | | |
| | | ATT | | |
| sele | Wild-type | AGTTGAATTGTAACTGGCAACATC | CCATGAAACTGGGAAGATGAAAGCAT | CAGGAAGAAATAATGGCAAAAACACCC |
| | | TTCTCTGTAATGCCCCCTGTGACC | TAGTTGAATTGTTACTGGCAACATCT | CCATTTCCTGTGGATATCTACAGCTCT |
| | | CATATTGTCTCGCTCTTTCCTTTA | TCTCTGTAATGCCCCCTGTGACCCAT | GTTTATAAAGGAAAGAGCGAGACAATA |
| | | TAAACAGAGCTGTAGATATCCACA | ATTGTCTCGCTCT | TGGGTCACAG |
| | | GGAAATGGGGGTGTTTTTGCCATT | | |
| | | ATTTCTTCCTGCCATGAAACTGGG | | |
| | | AAGATGAAAGCATT | | |
| sele | Ets | AGTTGAATTGTTACTGGCAACATC | CCATGAAACTGGGAAGATGAAAGCAT | CAtttGAAATAATGGCAAAAACACCCC |
| | mutant | TTCTCTGTAATGCCCCCTGTGACC | TAGTTGAATTGTTACTGGCAACATCT | CATTaaaTGTGGATATCTACAGCTCTG |
| | | CATATTGTCTCGCTCTTTaaaTTA | TCTCTGTAATGCCCCCTGTGACCCAT | TTTATAAtttAAAGAGCGAGACAATAT |
| | | TAAACAGAGCTGTAGATATCCACA | ATTGTCTCGCTCT | GGGTCACAG |
| | | tttAATGGGGGTGTTTTTGCCATT | | |
| | | ATTTCTaaaTGCCATGAAACTGGG | | |
| | | AAGATGAAAGCATT | | |
| sele | Sox | AGTTGAAggtggACTGGCAACATC | CCATGAAACTGGGAAGATGAAAGCAT | CAGGAAGtttattaGGCAAAAACACCC |
| | mutant | TTCTCTGTAATGCCCCCTGTGACC | TAGTTGAAggtggACTGGCAACATCT | CCATTTCCTGTGGATATCTACAGCTCT |
| | | CATAggtgaTCGCTCTTTCCTTTA | TCTCTGTAATGCCCCCCTGTGACCCA | GTTTATAAAGGAAGAGCGAtcaccTAT |
| | | TAAACAGAGCTGTAGATATCCACA | TAggtgaTCGCTCT | GGGTCACAG |
| | | GGAAATGGGGGTGTTTTTGCCATT | | |
| | | ATTTCTTCCTGCCATGAAACTGGG | | |
| | | AAGATGAAAGCATT | | |
| sele | NHR | AGTTGAATTGTTACTGGCAACATC | CCATGAAACTGGGAAGATGAAAGCAT | CAGGAAGAAATAATGGCAAAAACACCC |
| | mutant | TTCTCTGTAATGCCCCCTGattaa | TAGTTGAATTGTTACTGGCAACATCT | CCATTTCCTGTGGATATCTACAGCTCT |
| | | CATATTGTCTCGCTCTTTCCTTTA | TCTCTGTAATGCCCCCTGattaaCAT | GTTTATAAAGGAAAGAGCGAGACAATA |
| | | TAAACAGAGCTGTAGATATCCACA | ATTGTCTCGCTCT | TGttaatCAG |
| | | GGAAATGGGGGTGTTTTTGCCATT | | |
| | | ATTTCTTCCTGCCATGAACTGGGA | | |
| | | AtcTGAAAGCATT | | |
| sele | Control | AGTTGAATTGTTACTGGCAACATC | CCATGAAACTGGGAAtcTGAAAGCAT | CAGGAAGAAATAATGGCAAAAAgtCCC |
| | mutant | TTCTCTGTAATGCCCCCTGTGACC | TAGTTGAATTGTTACTGGCAACATCT | CCATTTCCTGTGGATATCTACctCTCT |
| | | CATATTGTCTCGCTCTTTCCTTTA | TCTCTGTAATGCCCCCTGTGACCCAT | GTTTATAAAGGAAAGAGCAGAGACAAT |
| | | TAAACAGAGagGTAGATATCCACA | ATTGTCTCGCTCT | ATGGGTCACAG |
| | | GGAAATGGGGGacTTTTTGCCATT | | |
| | | ATTTCTTCCTG | | |

In Table 8, lowercase letters indicate base pair changes used to disrupt transcription factor
binding motifs.

In Table 8, lowercase letters indicate base pair changes used to disrupt transcription factor binding motifs.

TABLE 9
Primers used for cloning and EMSA probe synthesis (“Forward” primers
disclosed as SEQ ID NOS 233-239 and “Reverse” primers disclosed
as SEQ ID NOS 240-246, all respectively, in order of appearance)

| Category | Primer Name | Forward | Reverse | Comment |
|---|---|---|---|---|
| Cloning | Nr2f2 | CGGGATCCatggca | CCGGAATTCCGGttgaattgccat | |
| | | atggtagtca gcacg | atatggc | |
| Probe | mrc1a site | ttta TGAAGCTTGTACCTTTCAT | CAAAAAGGAAATGAAAGGTACAAG | |
| synthesis | 1 wild-type | TTCCTTTTTG | CTTCAtaaa | |
| Probe | mrc1a site | TTTAattAGCagatTtaaTTTCAT | CAAAAAGGAAATGAAAttaAatct | 1st NHR site mutated |
| synthesis | 1 mutation | TTCCTTTTTG | GCTaatTAAA | like in vivo GFP |
| | | | | reporter experiment |
| Probe | mrc1a site | TTCATTTCCTTTTTGCTGAGCTTT | GAAAATAAAGCTCAGCAAAAAGGA | |
| synthesis | 2 wild-type | ATTTTC | AATGAA | |
| Probe | mrc1a site | TTCATTTCCTTTTTGCattaaTTT | GAAAATAAAttaatGCAAAAAGGA | 2nd NHR site mutated |
| synthesis | 2 mutation | ATTTTC | AATGAA | like in vivo GFP |
| | | | | reporter experiment |
| Probe | sele | GTAATGCCCCCTGTGACCCATATT | TATAAAGGAAAGAGCGAGACAATA | |
| synthesis | wild-type | GTCTCGCTCTTTCCTTTATA | TGGGTCACAGGGGGCATTAC | |
| Probe | sele | GTAATGCCCCCTGattaaCATATT | TATAAAGGAAAGAGCGAGACAATA | NHR site mutated |
| synthesis | mutation | GTCTCGCTCTTTCCTTTATA | TGttaatCAGGGGGCATTAC | like in vivo GFP |
| | | | | reporter experiment |

Table 9 shows primers used for cloning mouse Nr2f2 into the pGEX2TK vector and DNA probes from the zebrafish mrc1a and sele enhancers.

What is claimed herein is:

1. An engineered endothelial niche cell comprising one or more exogenous nucleic acid sequences encoding one or more transcription factors, wherein the one or more transcription factors are from the Ets family, the Sox family and/or the Nuclear Hormone Family.

2. The engineered endothelial niche cell of claim 1, wherein the one or more transcription factors comprise at least one of the transcription factors: ETV2, FLI1, ETS1, SOX18, SOX7, RXRA, or NR2F2.

3. A composition comprising the engineered endothelial niche cells of claim 1.

4. The composition of claim 2, wherein the composition is a therapeutic agent or the composition further comprises a pharmaceutically acceptable carrier.

5. The composition of claim 2, wherein the composition further comprises a culture dish, 3D cell system, or suspension system, and/or wherein the composition comprises a scaffold.

6. A method for culturing HSPCs, the method comprising culturing HSPCs in the presence of a population of the engineered endothelial niche cells of claim 1.

7. The method of claim 6, wherein the method is performed in vitro.

8. The method of claim 6, wherein the engineered endothelial niche cells secrete a factor that affects the growth and/or expansion of the HSPC cells.

9. The method of claim 6, wherein the HSPCs cultured in the presence of the engineered endothelial niche cells can be cultured for at least 3 days longer than HSPCs that are cultured in the absence of such engineered endothelial niche cells.

10. The method of claim 6, wherein the cells are cultured on a biologically compatible scaffold.

11. The method of claim 6, wherein the HSPCs cultured in the presence of the engineered endothelial niche cells have increased engraftment when administered to a subject compared to the engraftment of substantially similar HSPCs that were not cultured with engineered endothelial niche cells.

12. A method of treating a subject, the method comprising, transplanting a composition comprising HSPCs and a population of the engineered endothelial niche-cells of claim 1 into the subject.

13. A method for enhancing engraftment of HSPCs, the method comprising administering a composition comprising HSPCs and a population of the engineered endothelial niche cells of claim 1 to a subject in need thereof.

14. The method of claim 13, wherein engraftment of the HSPCs is increased by at least 10% compared to the engraftment of substantially similar HSPCs in the absence of engineered endothelial niche cells.

15. A co-culture comprising the engineered endothelial niche cells of claim 1 and HSPCs, optionally wherein the engineered endothelial niche cells are made by a method comprising expressing the one or more transcription factors in an endothelial cell.

16. A kit for culturing HSPCs, the kit comprising: a population of the engineered endothelial niche cells of claim 1, reagents and instructions for use thereof.

17. A kit for generating engineered endothelial niche cells comprising: a vector(s) comprising one or more exogenous nucleic acid sequences encoding one or more transcription factors of the Ets family, the Sox family or the nuclear hormone family and instructions for use thereof.

18. A method for generating an ectopic vascular niche, the method comprising: administering an engineered endothelial niche cell of claim 1 to a target site in a subject in need thereof.

19. A method for extra medullary hematopoiesis, the method comprising transplanting engineered-niche endothelial cells of claim 1 into a subject at a location outside of the bone marrow, thereby creating a synthetic niche, optionally wherein the engineered endothelial niche cells are made by a method comprising expressing the one or more transcription factors in an endothelial cell.

20. A vector comprising one or more exogenous nucleic acid sequences encoding one or more transcription factors of the Ets family, the Sox family or the nuclear hormone family operably linked to a promoter.


SEQUENCE LISTING (246 sequences)

SEQ ID NO: 1 - protein, 342 aa, Homo sapiens
MDLWNWDEASPQEVPPGNKLAGLEGAKLGFCFPDLALQGDTPTATAETCWKGTSSSLASFPQLDWGSALLHPEVPWGAEPDSQALPWSGDWTDMACTAWDSWSGASQTLGPAPLGPGPIPAAGSEGAAGQNCVPVAGEATSWSRAQAAGSNTSWDCSVGPDGDTYWGSGLGGEPRTDCTISWGGPAGPDCTTSWNPGLHAGGTTSLKRYQSSALTVCSEPSPQSDRASLARCPKTNHRGPIQLWQFLLELLHDGARSSCIRWTGNSREFQLCDPKEVARLWGERKRKPGMNYEKLSRGLRYYYRRDIVRKSGGRKYTYRFGGRVPSLAYPDCAGGGRGAETQ

SEQ ID NO: 2 - protein, 452 aa, Homo sapiens
MDGTIKEALSVVSDDQSLFDSAYGAAAHLPKADMTASGSPDYGQPHKINPLPPQQEWINQPVRVNVKREYDHMNGSRESPVDCSVSKCSKLVGGGESNPMNYNSYMDEKNGPPPPNMTTNERRVIVPADPTLWTQEHVRQWLEWAIKEYSLMEIDTSFFQNMDGKELCKMNKEDFLRATTLYNTEVLLSHLSYLRESSLLAYNTTSHTDQSSRLSVKEDPSYDSVRRGAWGNNMNSGLNKSPPLGGAQTISKNTEQRPQPDPYQILGPTSSRLANPGSGQIQLWQFLLELLSDSANASCITWEGTNGEFKMTDPDEVARRWGERKSKPNMNYDKLSRALRYYYDKNIMTKVHGKRYAYKFDFHGIAQALQPHPTESSMYKYPSDISYMPSYHAHQQKVNFVPPHPSSMPVTSSSFFGAASQYWTSPTGGIYPNPNVPRHPNTHVPSHLGSYY

SEQ ID NO: 3 - protein, 441 aa, Homo sapiens [sequence omitted]

SEQ ID NO: 4 - protein, 384 aa, Homo sapiens [sequence omitted]

SEQ ID NO: 5 - protein, 388 aa, Homo sapiens [sequence omitted]

SEQ ID NO: 6 - protein, 462 aa, Homo sapiens
MDTKHFLPLDFSTQVNSSLTSPTGRGSMAAPSLHPSLGPGIGSPGQLHSPISTLSSPINGMGPPFSVISSPMGPHSMSVPTTPTLGFSTGSPQLSSPMNPVSSSEDIKPPLGLNGVLKVPAHPSGNMASFTKHICAICGDRSSGKHYGVYSCEGCKGFFKRTVRKDLTYTCRDNKDCLIDKRQRNRCQYCRYQKCLAMGMKREAVQEERQRGKDRNENEVESTSSANEDMPVERILEAELAVEPKTETYVEANMGLNPSSPNDPVTNICQAADKQLFTLVEWAKRIPHFSELPLDDQVILLRAGWNELLIASFSHRSIAVKDGILLATGLHVHRNSAHSAGVGAIFDRVLTELVSKMRDMQMDKTELGCLRAIVLFNPDSKGLSNPAEVEALREKVYASLEAYCKHKYPEQPGRFAKLLLRLPALRSIGLKCLEHLFFFKLIGDTPIDTFLMEMLEAPHQMT

SEQ ID NO: 7 - protein, 414 aa, Homo sapiens
MAMVVSTWRDPQDEVPGSQGSQASQAPPVPGPPPGAPHTPQTPGQGGPASTPAQTAAGGQGGPGGPGSDKQQQQQHIECVVCGDKSSGKHYGQFTCEGCKSFFKRSVRRNLSYTCRANRNCPIDQHHRNQCQYCRLKKCLKVGMRREAVQRGRMPPTQPTHGQFALTNGDPLNCHSYLSGYISLLLRAEPYPTSRFGSQCMQPNNIMGIENICELAARMLFSAVEWARNIPFFPDLQITDQVALLRLTWSELFVLNAAQCSMPLHVAPLLAAAGLHASPMSADRVVAFMDHIRIFQEQVEKLKALHVDSAEYSCLKAIVLFTSDACGLSDVAHVESLQEKSQCALEEYVRSQYPNQPTRFGKLLLRLPSLRTVSSSVIEQLFFVRLVGKTPIETLIRDMLLSGSSFNWPYMAIQ

SEQ ID NO: 8 - protein, 362 aa, Xenopus tropicalis
MHRPEPSYCREEPTPCQGVNSTWVPPADTVPETSPTPSSPPAPDSPTPSPQPGYGYSPCEEKPGDPRIRRPMNAFMVWAKDERKRLAQQNPDLHNAVLSKMLGQSWKNLSSAEKRPFVEEAERLRVQHLQDHPNYKYRPRRKKQAKKLKRVDPSPLLRNEGYRGQAMANLSHFRDLHPLGGSGDLESYGLPTPEMSPLDVVEPSEPAFFPPHMREEADPGPFRTYQHGVDFGQEKTLREISLPYSSSPSHMGGFLRTPTASAFYYNPHGGSPACTPLGQLSPPPEAPALEAMDHLGPAELWGDFDRNEFDQYLNMSRTQGPGYPFPMSKLGAPRTIPCEESSLISALSDASTAMYYTPCITG

SEQ ID NO: 9 - protein, 428 aa, Danio rerio
MAMVVWRGSQDDVAETHGTLSSQTQGGLSLPTPQPGQLGLTASQVAPPTPQTPVQGPPNNNNNTQSTPTNQTTQSQSEKQQPQHIECVVCGDKSSGKHYGQFTCEGCKSFFKRSVRRNLTYTCRANRNCPIDQHHRNQCQYCRLKKCLKVGMRREVSLFTAAVQRGRMPPTQPHHGQFALTNGDPLHCHSYLSGYISLLLRAEPYPTSRYGSQCMQPNNIMGIENICELAARMLFSAVEWARNIPFFPDLQITDQVALLRLTWSELFVLNAAQCSMPLHVAPLLAAAGLHASPMSADRVVAFMDHIRIFQEQVEKLKALHVDSAEYSCLKAIVLFTSDACGLSDVAHVESLQEKSQCALEEYVRSQYPNQPTRFGKLLLRLPSLRTVSSSVIEQLFFVRLVGKTPIETLIRDMLLSGSSFNWPYMSIQ

SEQ ID NO: 10 - DNA, 21 bp, synthetic construct
  misc_feature 1..21: note = Description of Artificial Sequence: Synthetic oligonucleotide
  source 1..21: mol_type = other DNA
cattaaccctcataaagggaa

SEQ ID NO: 11 - DNA, 20 bp, synthetic construct
  misc_feature 1..20: note = Description of Artificial Sequence: Synthetic oligonucleotide
  source 1..20: mol_type = other DNA
taatacgactcactataggg

SEQ ID NO: 12 - DNA, 125 bp, Danio rerio
  source 1..125: mol_type = unassigned DNA
tgaagcttgtacctttcatttcctttttgctgagctttattttctctagaattgccattgtgtttccattctagcaaatcagcatttttttttcagctgaaagaaaaataccaggaactgagagc

SEQ ID NO: 13 - DNA, 158 bp, Danio rerio
  source 1..158: mol_type = unassigned DNA
ccatgaaactgggaagatgaaagcattagttgaattgttactggcaacatcttctctgtaatgccccctgtgacccatattgtctcgctctttcctttataaacagagctgtagatatccacaggaaatgggggtgtttttgccattatttcttcctg

SEQ ID NO: 14 - DNA, 820 bp, synthetic construct
  misc_feature 1..820: note = Description of Artificial Sequence: Synthetic polynucleotide
  source 1..820: mol_type = other DNA
ttgtgggtgctatttctgtaatgaggaagctggtacaacactgagcaaacacactaaacagagtggcatcctagaaggttcaaggtctcacgaaatttcataatgagaaattcctggagagcttaacaataatggcttttaaacaaagcaaatataataccaccctatttttaaattcaattaaatggtattggacatatattattgtcaaatacgtcttctgatctaacgcttgttattttggtatgagaaaattttagtttatggtttattttattaacattttatgggagtcatatgcttatggcatgtttacatgatgttctctttccatgaaactgggaagatgaaagcattagttgaattgttactggcaacatcttctctgtaatgccccctgtgacccatattgtctcgctctttcctttataaacagagctgtagatatccacaggaaatgggggtgtttttgccattatttcttcctgctaaagttaatcaggtggtccaaaaattggtataattcattccttcatttgcttttcggcttagtccatcagttaatcaggggtcgccacagtggaatgaacctatatccaatgtatgttttacgcagcggatgcccttccagctgcaacccaacactgggaaacattttagcttactcaattcacctataccacgtctttgggcttgtgggggaaaccagagcacccggaggaaacccatgcaaacacggggagaacatgaaaactccaaacagaaacgctaactgacccagccagggctcgaaccagcaagcttcctgctgtgaggcgat

SEQ ID NO: 15 - DNA, 405 bp, synthetic construct
  misc_feature 1..405: note = Description of Artificial Sequence: Synthetic polynucleotide
  source 1..405: mol_type = other DNA
ctgtttcctggcagctctggcccgtgtctcaaaatctctgatgttacattgcacaagataaaaatatatcatcatgaacaataaaactgtctgcttacataaacagtaatacaaggggtgttatgagccatattcatttatgaagcttgtacctttcatttcctttttgctgagctttattttctctagaattgccattgtgtttccattctagcaaatcagcatttttttttcagctgaaagaaaaataccaggaactgagagcgcggctcctggcagctctggcccgtgtctcaaaatctctgatgttacattgcacaagataaaaatatatcatcatgaacaataaaactgtctgcttacataaacagtaatacaaggggtgttatgagccatattcaacgg

SEQ ID NO: 16 - DNA, 43 bp, synthetic construct
  misc_feature 1..43: note = Description of Artificial Sequence: Synthetic primer
  source 1..43: mol_type = other DNA
cattaaccctcataaagggaagctccatagtatcgtctgaacc

SEQ ID NO: 17 - DNA, 41 bp, synthetic construct
  misc_feature 1..41: note = Description of Artificial Sequence: Synthetic primer
  source 1..41: mol_type = other DNA
cattaaccctcataaagggaagggagttcttcgggtgactg

SEQ ID NO: 18 - DNA, 41 bp, synthetic construct
  misc_feature 1..41: note = Description of Artificial Sequence: Synthetic primer
  source 1..41: mol_type = other DNA
cattaaccctcataaagggaatgtgtgatctcaactgcgct

SEQ ID NO: 19 - DNA, 41 bp, synthetic construct
  misc_feature 1..41: note = Description of Artificial Sequence: Synthetic primer
  source 1..41: mol_type = other DNA
cattaaccctcataaagggaaccagcaaaccccatggatct

SEQ ID NO: 20 - DNA, 41 bp, synthetic construct
  misc_feature 1..41: note = Description of Artificial Sequence: Synthetic primer
  source 1..41: mol_type = other DNA
cattaaccctcataaagggaacgactggagaaccaagggac

SEQ ID NO: 21 - DNA, 41 bp, synthetic construct
  misc_feature 1..41: note = Description of Artificial Sequence: Synthetic primer
  source 1..41: mol_type = other DNA
cattaaccctcataaagggaaccatgcaacagaggaagggt

SEQ ID NO: 22 - DNA, 41 bp, synthetic construct
  misc_feature 1..41: note = Description of Artificial Sequence: Synthetic primer
  source 1..41: mol_type = other DNA
cattaaccctcataaagggaaaagttccgcaggatggactg

SEQ ID NO: 23 - DNA, 41 bp, synthetic construct
  misc_feature 1..41: note = Description of Artificial Sequence: Synthetic primer
  source 1..41: mol_type = other DNA
cattaaccctcataaagggaaagttatgccagctcagtggg

SEQ ID NO: 24 - DNA, 41 bp, synthetic construct
  misc_feature 1..41: note = Description of Artificial Sequence: Synthetic primer
  source 1..41: mol_type = other DNA
cattaaccctcataaagggaacttcccacagcagcacaaac

SEQ ID NO: 25 - DNA, 41 bp, synthetic construct
  misc_feature 1..41: note = Description of Artificial Sequence: Synthetic primer
  source 1..41: mol_type = other DNA
cattaaccctcataaagggaagaatttgctcgcatgagggg

SEQ ID NO: 26 - DNA, 41 bp, synthetic construct
  misc_feature 1..41: note = Description of Artificial Sequence: Synthetic primer
  source 1..41: mol_type = other DNA
cattaaccctcataaagggaaatggaggtctaccacaggct

SEQ ID NO: 27 - DNA, 41 bp, synthetic construct
  misc_feature 1..41: note = Description of Artificial Sequence: Synthetic primer
  source 1..41: mol_type = other DNA
cattaaccctcataaagggaattcggctttaacctgtgcgt

SEQ ID NO: 28 - DNA, 40 bp, synthetic construct
  misc_feature 1..40: note = Description of Artificial Sequence: Synthetic primer
  source 1..40: mol_type = other DNA
cattaaccctcataaagggaaagttagaatgggcgccacc

SEQ ID NO: 29 - DNA, 42 bp, synthetic construct
  misc_feature 1..42: note = Description of Artificial Sequence: Synthetic primer
  source 1..42: mol_type = other DNA
cattaaccctcataaagggaaaacttgagccaccgaggattt

SEQ ID NO: 30 - DNA, 40 bp, synthetic construct
  misc_feature 1..40: note = Description of Artificial Sequence: Synthetic primer
  source 1..40: mol_type = other DNA
cattaaccctcataaagggaagctacagtctgcgtagcat

SEQ ID NO: 31 - DNA, 41 bp, synthetic construct
  misc_feature 1..41: note = Description of Artificial Sequence: Synthetic primer
  source 1..41: mol_type = other DNA
cattaaccctcataaagggaatacccaatggttcgagtggc

SEQ ID NO: 32 - DNA, 41 bp, synthetic construct
  misc_feature 1..41: note = Description of Artificial Sequence: Synthetic primer
  source 1..41: mol_type = other DNA
cattaaccctcataaagggaagtgtcccctcatcaatgcca

SEQ ID NO: 33 - DNA, 41 bp, synthetic construct
  misc_feature 1..41: note = Description of Artificial Sequence: Synthetic primer
  source 1..41: mol_type = other DNA
cattaaccctcataaagggaatgcccagcccttgataatct

SEQ ID NO: 34 - DNA, 41 bp, synthetic construct
  misc_feature 1..41: note = Description of Artificial Sequence: Synthetic primer
  source 1..41: mol_type = other DNA
cattaaccctcataaagggaaaggcagcactggactttagc

SEQ ID NO: 35 - DNA, 44 bp, synthetic construct
  misc_feature 1..44: note = Description of Artificial Sequence: Synthetic primer
  source 1..44: mol_type = other DNA
cattaaccctcataaagggaaacaaagagatctgcattccaagc

SEQ ID NO: 36 - DNA, 43 bp, synthetic construct
  misc_feature 1..43: note = Description of Artificial Sequence: Synthetic primer
  source 1..43: mol_type = other DNA
cattaaccctcataaagggaaaaggcgtactatgtcctcaggc

SEQ ID NO: 37 - DNA, 41 bp, synthetic construct
  misc_feature 1..41: note = Description of Artificial Sequence: Synthetic primer
  source 1..41: mol_type = other DNA
cattaaccctcataaagggaattgtggattacggggttcgg

SEQ ID NO: 38 - DNA, 41 bp, synthetic construct
  misc_feature 1..41: note = Description of Artificial Sequence: Synthetic primer
  source 1..41: mol_type = other DNA
cattaaccctcataaagggaagagctttactctgctggcga

SEQ ID NO: 39 - DNA, 41 bp, synthetic construct
  misc_feature 1..41: note = Description of Artificial Sequence: Synthetic primer
  source 1..41: mol_type = other DNA
cattaaccctcataaagggaactgttttcccccaccagtga

SEQ ID NO: 40 - DNA, 46 bp, synthetic construct
  misc_feature 1..46: note = Description of Artificial Sequence: Synthetic primer
  source 1..46: mol_type = other DNA
cattaaccctcataaagggaatgggatttatttgtaactacacgga

SEQ ID NO: 41 - DNA, 41 bp, synthetic construct
  misc_feature 1..41: note = Description of Artificial Sequence: Synthetic primer
  source 1..41: mol_type = other DNA
cattaaccctcataaagggaaccaggaagcgcaaagaacag

SEQ ID NO: 42 - DNA, 41 bp, synthetic construct
  misc_feature 1..41: note = Description of Artificial Sequence: Synthetic primer
  source 1..41: mol_type = other DNA
cattaaccctcataaagggaagcagcaaacgactgctacaa

SEQ ID NO: 43 - DNA, 41 bp, synthetic construct
  misc_feature 1..41: note = Description of Artificial Sequence: Synthetic primer
  source 1..41: mol_type = other DNA
cattaaccctcataaagggaatcatccacacctgagacgca

SEQ ID NO: 44 - DNA, 41 bp, synthetic construct
  misc_feature 1..41: note = Description of Artificial Sequence: Synthetic primer
  source 1..41: mol_type = other DNA
cattaaccctcataaagggaaatccgactgctggaatggac

SEQ ID NO: 45 - DNA, 41 bp, synthetic construct
  misc_feature 1..41: note = Description of Artificial Sequence: Synthetic primer
  source 1..41: mol_type = other DNA
cattaaccctcataaagggaagtgctggtcaacatgtacgc

SEQ ID NO: 46 - DNA, 42 bp, synthetic construct
  misc_feature 1..42: note = Description of Artificial Sequence: Synthetic primer
  source 1..42: mol_type = other DNA
cattaaccctcataaagggaatccactgggaaaagctggaat

SEQ ID NO: 47 - DNA, 39 bp, synthetic construct
  misc_feature 1..39: note = Description of Artificial Sequence: Synthetic primer
  source 1..39: mol_type = other DNA
cattaaccctcataaagggaaatggggctgacggatacc

SEQ ID NO: 48 - DNA, 41 bp, synthetic construct
  misc_feature 1..41: note = Description of Artificial Sequence: Synthetic primer
  source 1..41: mol_type = other DNA
cattaaccctcataaagggaattgtccagactaccaaggcg

SEQ ID NO: 49 - DNA, 41 bp, synthetic construct
  misc_feature 1..41: note = Description of Artificial Sequence: Synthetic primer
  source 1..41: mol_type = other DNA
cattaaccctcataaagggaaatccaaactgaggccggaag

SEQ ID NO: 50 - DNA, 41 bp, synthetic construct
  misc_feature 1..41: note = Description of Artificial Sequence: Synthetic primer
  source 1..41: mol_type = other DNA
cattaaccctcataaagggaaggcccaagcatggcagaaac

SEQ ID NO: 51 - DNA, 41 bp, synthetic construct
  misc_feature 1..41: note = Description of Artificial Sequence: Synthetic primer
  source 1..41: mol_type = other DNA
cattaaccctcataaagggaaacatgggacatggagcgaag

SEQ ID NO: 52 - DNA, 41 bp, synthetic construct
  misc_feature 1..41: note = Description of Artificial Sequence: Synthetic primer
  source 1..41: mol_type = other DNA
cattaaccctcataaagggaagactctctgcggatgtcagg

SEQ ID NO: 53 - DNA, 41 bp, synthetic construct
  misc_feature 1..41: note = Description of Artificial Sequence: Synthetic primer
  source 1..41: mol_type = other DNA
cattaaccctcataaagggaacatgtccacccctgaagctg

SEQ ID NO: 54 - DNA, 41 bp, synthetic construct
  misc_feature 1..41: note = Description of Artificial Sequence: Synthetic primer
  source 1..41: mol_type = other DNA
cattaaccctcataaagggaaggcgtctctttttctgctgc

SEQ ID NO: 55 - DNA, 41 bp, synthetic construct
  misc_feature 1..41: note = Description of Artificial Sequence: Synthetic primer
  source 1..41: mol_type = other DNA
cattaaccctcataaagggaaccctagtgtccgaggtctca

SEQ ID NO: 56 - DNA, 41 bp, synthetic construct
  misc_feature 1..41: note = Description of Artificial Sequence: Synthetic primer
  source 1..41: mol_type = other DNA
cattaaccctcataaagggaagataccgtgctgggcgatta

SEQ ID NO: 57 - DNA, 41 bp, synthetic construct
  misc_feature 1..41: note = Description of Artificial Sequence: Synthetic primer
  source 1..41: mol_type = other DNA
cattaaccctcataaagggaatcccggtgcaaaatctgagg

SEQ ID NO: 58 - DNA, 41 bp, synthetic construct
  misc_feature 1..41: note = Description of Artificial Sequence: Synthetic primer
  source 1..41: mol_type = other DNA
cattaaccctcataaagggaagtttattgctggcgcctacg

SEQ ID NO: 59 - DNA, 41 bp, synthetic construct
  misc_feature 1..41: note = Description of Artificial Sequence: Synthetic primer
  source 1..41: mol_type = other DNA
cattaaccctcataaagggaagctgccgaacaacgaaacat

SEQ ID NO: 60 - DNA, 41 bp, synthetic construct
  misc_feature 1..41: note = Description of Artificial Sequence: Synthetic primer
  source 1..41: mol_type = other DNA
cattaaccctcataaagggaaagtacactccggacccaaga

SEQ ID NO: 61 - DNA, 41 bp, synthetic construct
  misc_feature 1..41: note = Description of Artificial Sequence: Synthetic primer
  source 1..41: mol_type = other DNA
cattaaccctcataaagggaatccagagcgattcagcatca

SEQ ID NO: 62 - DNA, 42 bp, synthetic construct
  misc_feature 1..42: note = Description of Artificial Sequence: Synthetic primer
  source 1..42: mol_type = other DNA
cattaaccctcataaagggaaagtctcctacttcgagtggct

SEQ ID NO: 63 - DNA, 43 bp, synthetic construct
  misc_feature 1..43: note = Description of Artificial Sequence: Synthetic primer
  source 1..43: mol_type = other DNA
cattaaccctcataaagggaaaactgttctttctcccggtccc

SEQ ID NO: 64 - DNA, 41 bp, synthetic construct
  misc_feature 1..41: note = Description of Artificial Sequence: Synthetic primer
  source 1..41: mol_type = other DNA
cattaaccctcataaagggaaactggtcaaccacctcctct

SEQ ID NO: 65 - DNA, 41 bp, synthetic construct
  misc_feature 1..41: note = Description of Artificial Sequence: Synthetic primer
  source 1..41: mol_type = other DNA
cattaaccctcataaagggaatgcctttcaaagtggaccgt

SEQ ID NO: 66 - DNA, 41 bp, synthetic construct
  misc_feature 1..41: note = Description of Artificial Sequence: Synthetic primer
  source 1..41: mol_type = other DNA
cattaaccctcataaagggaaagcctgttcctggaccattg

SEQ ID NO: 67 - DNA, 41 bp, synthetic construct
  misc_feature 1..41: note = Description of Artificial Sequence: Synthetic primer
  source 1..41: mol_type = other DNA
cattaaccctcataaagggaaggttcgccagaaggacaaga

SEQ ID NO: 68 - DNA, 41 bp, synthetic construct
  misc_feature 1..41: note = Description of Artificial Sequence: Synthetic primer
  source 1..41: mol_type = other DNA
cattaaccctcataaagggaaaagctgtcatcaaagccgga

SEQ ID NO: 69 - DNA, 41 bp, synthetic construct
  misc_feature 1..41: note = Description of Artificial Sequence: Synthetic primer
  source 1..41: mol_type = other DNA
cattaaccctcataaagggaatgaaagccctgaacgagacc

SEQ ID NO: 70 - DNA, 41 bp, synthetic construct
  misc_feature 1..41: note = Description of Artificial Sequence: Synthetic primer
  source 1..41: mol_type = other DNA
cattaaccctcataaagggaaaggagtttggcttcgaccag

SEQ ID NO: 71 - DNA, 41 bp, synthetic construct
  misc_feature 1..41: note = Description of Artificial Sequence: Synthetic primer
  source 1..41: mol_type = other DNA
cattaaccctcataaagggaatcgctctgatgctcagcttg

SEQ ID NO: 72 - DNA, 43 bp, synthetic construct
  misc_feature 1..43: note = Description of Artificial Sequence: Synthetic primer
  source 1..43: mol_type = other DNA
cattaaccctcataaagggaatacacattttctgccccactga

SEQ ID NO: 73 - DNA, 41 bp, synthetic construct
  misc_feature 1..41: note = Description of Artificial Sequence: Synthetic primer
  source 1..41: mol_type = other DNA
cattaaccctcataaagggaactcaccctcggtccagaact

SEQ ID NO: 74 - DNA, 41 bp, synthetic construct
  misc_feature 1..41: note = Description of Artificial Sequence: Synthetic primer
  source 1..41: mol_type = other DNA
cattaaccctcataaagggaaacagccatcagttcctctgc

SEQ ID NO: 75 - DNA, 41 bp, synthetic construct
  misc_feature 1..41: note = Description of Artificial Sequence: Synthetic primer
  source 1..41: mol_type = other DNA
cattaaccctcataaagggaaggaagccactcctgatacgg

SEQ ID NO: 76 - DNA, 41 bp, synthetic construct
  misc_feature 1..41: note = Description of Artificial Sequence: Synthetic primer
  source 1..41: mol_type = other DNA
cattaaccctcataaagggaaggtcacgcaccaaatgaacc

SEQ ID NO: 77 - DNA, 41 bp, synthetic construct
  misc_feature 1..41: note = Description of Artificial Sequence: Synthetic primer
  source 1..41: mol_type = other DNA
cattaaccctcataaagggaatctgcatctcgcaagctgat

SEQ ID NO: 78 - DNA, 41 bp, synthetic construct
  misc_feature 1..41: note = Description of Artificial Sequence: Synthetic primer
  source 1..41: mol_type = other DNA
cattaaccctcataaagggaaagaactacgacagcgactgc

SEQ ID NO: 79 - DNA, 46 bp, synthetic construct
  misc_feature 1..46: note = Description of Artificial Sequence: Synthetic primer
  source 1..46: mol_type = other DNA
cattaaccctcataaagggaaacagactctgtacgtttgaatgcgt

SEQ ID NO: 80 - DNA, 43 bp, synthetic construct
  misc_feature 1..43: note = Description of Artificial Sequence: Synthetic primer
  source 1..43: mol_type = other DNA
cattaaccctcataaagggaatatgactgcagtggtgaagacc

SEQ ID NO: 81 - DNA, 40 bp, synthetic construct
  misc_feature 1..40: note = Description of Artificial Sequence: Synthetic primer
  source 1..40: mol_type = other DNA
cattaaccctcataaagggaacagacccgtctctgtggtc

SEQ ID NO: 82 - DNA, 41 bp, synthetic construct
  misc_feature 1..41: note = Description of Artificial Sequence: Synthetic primer
  source 1..41: mol_type = other DNA
cattaaccctcataaagggaaacccccgaacaacaataaca

SEQ ID NO: 83 - DNA, 41 bp, synthetic construct
  misc_feature 1..41: note = Description of Artificial Sequence: Synthetic primer
  source 1..41: mol_type = other DNA
cattaaccctcataaagggaatatagcccttcgttccccca

SEQ ID NO: 84 - DNA, 43 bp, synthetic construct
  misc_feature 1..43: note = Description of Artificial Sequence: Synthetic primer
  source 1..43: mol_type = other DNA
cattaaccctcataaagggaatccttggacgctgtggaccaac

SEQ ID NO: 85 - DNA, 40 bp, synthetic construct
  misc_feature 1..40: note = Description of Artificial Sequence: Synthetic primer
  source 1..40: mol_type = other DNA
taatacgactcactatagggaaaccattgccattttgcca

SEQ ID NO: 86 - DNA, 40 bp, synthetic construct
  misc_feature 1..40: note = Description of Artificial Sequence: Synthetic primer
  source 1..40: mol_type = other DNA
taatacgactcactataggggcttgcaacaaaaagcgcag

SEQ ID NO: 87 - DNA, 42 bp, synthetic construct
  misc_feature 1..42: note = Description of Artificial Sequence: Synthetic primer
  source 1..42: mol_type = other DNA
taatacgactcactatagggggtgcaatctagtctgatcggt

SEQ ID NO: 88 - DNA, 40 bp, synthetic construct
  misc_feature 1..40: note = Description of Artificial Sequence: Synthetic primer
  source 1..40: mol_type = other DNA
taatacgactcactatagggaaccgagtacaggacacacg

SEQ ID NO: 89 - DNA, 40 bp, synthetic construct
  misc_feature 1..40: note = Description of Artificial Sequence: Synthetic primer
  source 1..40: mol_type = other DNA
taatacgactcactatagggtggaggctaatcgagtgtgc

SEQ ID NO: 90 - DNA, 40 bp, synthetic construct
  misc_feature 1..40: note = Description of Artificial Sequence: Synthetic primer
  source 1..40: mol_type = other DNA
taatacgactcactatagggtactgggcgggtctccttta

SEQ ID NO: 91 - DNA, 43 bp, synthetic construct
  misc_feature 1..43: note = Description of Artificial Sequence: Synthetic primer
  source 1..43: mol_type = other DNA
taatacgactcactatagggtcgcttgtgtgatcaagtatgac

SEQ ID NO: 92 - DNA, 40 bp, synthetic construct
  misc_feature 1..40: note = Description of Artificial Sequence: Synthetic primer
  source 1..40: mol_type = other DNA
taatacgactcactatagggggcctccccaagaaaccatt

SEQ ID NO: 93 - DNA, 43 bp, synthetic construct
  misc_feature 1..43: note = Description of Artificial Sequence: Synthetic primer
  source 1..43: mol_type = other DNA
taatacgactcactataggggaaagttgttgttgaagtgaacg

SEQ ID NO: 94 - DNA, 40 bp, synthetic construct
  misc_feature 1..40: note = Description of Artificial Sequence: Synthetic primer
  source 1..40: mol_type = other DNA
taatacgactcactatagggcggcagtggccaacaaatag

SEQ ID NO: 95 - DNA, 40 bp, synthetic construct
  misc_feature 1..40: note = Description of Artificial Sequence: Synthetic primer
  source 1..40: mol_type = other DNA
taatacgactcactatagggagtgcaggtatgtgtccgtg

SEQ ID NO: 96 - DNA, 40 bp, synthetic construct
  misc_feature 1..40: note = Description of Artificial Sequence: Synthetic primer
  source 1..40: mol_type = other DNA
taatacgactcactatagggcctgacgcgagtagtgttgt

SEQ ID NO: 97 - DNA, 40 bp, synthetic construct
  misc_feature 1..40: note = Description of Artificial Sequence: Synthetic primer
  source 1..40: mol_type = other DNA
taatacgactcactatagggggcaagaccactggcatttg

SEQ ID NO: 98 - DNA, 40 bp, synthetic construct
  misc_feature 1..40: note = Description of Artificial Sequence: Synthetic primer
  source 1..40: mol_type = other DNA
taatacgactcactatagggccctaactccagcacacact

SEQ ID NO: 99 - DNA, 42 bp, synthetic construct
  misc_feature 1..42: note = Description of Artificial Sequence: Synthetic primer
  source 1..42: mol_type = other DNA
taatacgactcactatagggtggaagcagctctaagtgacag

SEQ ID NO: 100 - DNA, 44 bp, synthetic construct
  misc_feature 1..44: note = Description of Artificial Sequence: Synthetic primer
  source 1..44: mol_type = other DNA
taatacgactcactataggggcttaggtgatcaattttgggaca

SEQ ID NO: 101 - DNA, 40 bp, synthetic construct
  misc_feature 1..40: note = Description of Artificial Sequence: Synthetic primer
  source 1..40: mol_type = other DNA
taatacgactcactatagggacggcattccacaaaccaga

SEQ ID NO: 102 - DNA, 43 bp, synthetic construct
  misc_feature 1..43: note = Description of Artificial Sequence: Synthetic primer
  source 1..43: mol_type = other DNA
taatacgactcactatagggacccaaactgactttatatgtgc

SEQ ID NO: 103 - DNA, 45 bp, synthetic construct
  misc_feature 1..45: note = Description of Artificial Sequence: Synthetic primer
  source 1..45: mol_type = other DNA
taatacgactcactatagggaggtacaaatgcaagtacaacactg

SEQ ID NO: 104 - DNA, 41 bp, synthetic construct
  misc_feature 1..41: note = Description of Artificial Sequence: Synthetic primer
  source 1..41: mol_type = other DNA
taatacgactcactatagggagcctgtcagctcactttatt

SEQ ID NO: 105 - DNA, 40 bp, synthetic construct
  misc_feature 1..40: note = Description of Artificial Sequence: Synthetic primer
  source 1..40: mol_type = other DNA
taatacgactcactatagggcgccgttctataatgcaccg

SEQ ID NO: 106 - DNA, 39 bp, synthetic construct
  misc_feature 1..39: note = Description of Artificial Sequence: Synthetic primer
  source 1..39: mol_type = other DNA
taatacgactcactatagggaaagagagctgcaccgact

SEQ ID NO: 107 - DNA, 42 bp, synthetic construct
  misc_feature 1..42: note = Description of Artificial Sequence: Synthetic primer
  source 1..42: mol_type = other DNA
taatacgactcactatagggacaaatgatgtctgtctccgct

SEQ ID NO: 108 - DNA, 40 bp, synthetic construct
  misc_feature 1..40: note = Description of Artificial Sequence: Synthetic primer
  source 1..40: mol_type = other DNA
taatacgactcactatagggcaaaggattggcagggacca

SEQ ID NO: 109 - DNA, 44 bp, synthetic construct
  misc_feature 1..44: note = Description of Artificial Sequence: Synthetic primer
  source 1..44: mol_type = other DNA
taatacgactcactatagggttttaagcacatttctgaagcaca

SEQ ID NO: 110 - DNA, 41 bp, synthetic construct
  misc_feature 1..41: note = Description of Artificial Sequence: Synthetic primer
  source 1..41: mol_type = other DNA
taatacgactcactatagggacattaggcgggcaaaacaaa

SEQ ID NO: 111 - DNA, 40 bp, synthetic construct
  misc_feature 1..40: note = Description of Artificial Sequence: Synthetic primer
  source 1..40: mol_type = other DNA
taatacgactcactatagggcccacttcgctgctctttac

SEQ ID NO: 112 - DNA, 39 bp, synthetic construct
  misc_feature 1..39: note = Description of Artificial Sequence: Synthetic primer
  source 1..39: mol_type = other DNA
taatacgactcactatagggacaccttaaaaccgcagcc

SEQ ID NO: 113 - DNA, 41 bp, synthetic construct
  misc_feature 1..41: note = Description of Artificial Sequence: Synthetic primer
  source 1..41: mol_type = other DNA
taatacgactcactataggggctttgaggagcttaagaggc

SEQ ID NO: 114 - DNA, 40 bp, synthetic construct
  misc_feature 1..40: note = Description of Artificial Sequence: Synthetic primer
  source 1..40: mol_type = other DNA
taatacgactcactatagggcgtcacttttcacccccaga

SEQ ID NO: 115 - DNA, 40 bp, synthetic construct
  misc_feature 1..40: note = Description of Artificial Sequence: Synthetic primer
  source 1..40: mol_type = other DNA
taatacgactcactatagggtttcagatgcagcacaggca

SEQ ID NO: 116 - DNA, 40 bp, synthetic construct
  misc_feature 1..40: note = Description of Artificial Sequence: Synthetic primer
  source 1..40: mol_type = other DNA
taatacgactcactataggggcacaggaaacgcacatgat

SEQ ID NO: 117 - DNA, 40 bp, synthetic construct
  misc_feature 1..40: note = Description of Artificial Sequence: Synthetic primer
  source 1..40: mol_type = other DNA
taatacgactcactatagggttacttcactgccagtcggc

SEQ ID NO: 118 - DNA, 45 bp, synthetic construct
  misc_feature 1..45: note = Description of Artificial Sequence: Synthetic primer
  source 1..45: mol_type = other DNA
taatacgactcactatagggagaaaatacagtgcatacatgtcaa

SEQ ID NO: 119 - DNA, 43 bp, synthetic construct
  misc_feature 1..43: note = Description of Artificial Sequence: Synthetic primer
  source 1..43: mol_type = other DNA
taatacgactcactatagggcctacaagcctcattcagacagt

SEQ ID NO: 120 - DNA, 43 bp, synthetic construct
  misc_feature 1..43: note = Description of Artificial Sequence: Synthetic primer
  source 1..43: mol_type = other DNA
taatacgactcactatagggacactagaaaaccgatcgtgtca

SEQ ID NO: 121 - DNA, 40 bp, synthetic construct
  misc_feature 1..40: note = Description of Artificial Sequence: Synthetic primer
  source 1..40: mol_type = other DNA
taatacgactcactatagggggtcatgcccttttggcttg

SEQ ID NO: 122 - DNA, 40 bp, synthetic construct
  misc_feature 1..40: note = Description of Artificial Sequence: Synthetic primer
  source 1..40: mol_type = other DNA
taatacgactcactataggggggttctgttgtggagtgct

SEQ ID NO: 123 - DNA, 40 bp, synthetic construct
  misc_feature 1..40: note = Description of Artificial Sequence: Synthetic primer
  source 1..40: mol_type = other DNA
taatacgactcactatagggtgacgcttaaacagagcggt

SEQ ID NO: 124 - DNA, 40 bp, synthetic construct
  misc_feature 1..40: note = Description of Artificial Sequence: Synthetic primer
  source 1..40: mol_type = other DNA
taatacgactcactatagggtttcccctgtgtggatgagc

SEQ ID NO: 125 - DNA, 42 bp, synthetic construct
  misc_feature 1..42: note = Description of Artificial Sequence: Synthetic primer
  source 1..42: mol_type = other DNA
taatacgactcactatagggccaccactcactcattcagaca

SEQ ID NO: 126 - DNA, 41 bp, synthetic construct
  misc_feature 1..41: note = Description of Artificial Sequence: Synthetic primer
  source 1..41: mol_type = other DNA
taatacgactcactatagggagtcatgcacaaaatctgcgg

SEQ ID NO: 127 - DNA, 40 bp, synthetic construct
  misc_feature 1..40: note = Description of Artificial Sequence: Synthetic primer
  source 1..40: mol_type = other DNA
taatacgactcactatagggttcttggggtagttgcagcc

SEQ ID NO: 128 - DNA, 40 bp, synthetic construct
  misc_feature 1..40: note = Description of Artificial Sequence: Synthetic primer
  source 1..40: mol_type = other DNA
taatacgactcactatagggtaggacgcgtcattgtgctt

SEQ ID NO: 129 - DNA, 41 bp, synthetic construct
  misc_feature 1..41: note = Description of Artificial Sequence: Synthetic primer
  source 1..41: mol_type = other DNA
taatacgactcactatagggcaccagaaagaatgtcaccgt

SEQ ID NO: 130 - DNA, 40 bp, synthetic construct
  misc_feature 1..40: note = Description of Artificial Sequence: Synthetic primer
  source 1..40: mol_type = other DNA
taatacgactcactatagggcagtccctcagtattccccg

SEQ ID NO: 131 - DNA, 40 bp, synthetic construct
  misc_feature 1..40: note = Description of Artificial Sequence: Synthetic primer
  source 1..40: mol_type = other DNA
taatacgactcactatagggacgcctgagattcatcctgc

SEQ ID NO: 132 - DNA, 40 bp, synthetic construct
  misc_feature 1..40: note = Description of Artificial Sequence: Synthetic primer
  source 1..40: mol_type = other DNA
taatacgactcactatagggcctctggctgaacaggaagg

SEQ ID NO: 133 - DNA, 40 bp, synthetic construct
  misc_feature 1..40: note = Description of Artificial Sequence: Synthetic primer
  source 1..40: mol_type = other DNA
taatacgactcactatagggacacaatcaggcaaggtctc

SEQ ID NO: 134 - DNA, 40 bp, synthetic construct
  misc_feature 1..40: note = Description of Artificial Sequence: Synthetic primer
  source 1..40: mol_type = other DNA
taatacgactcactatagggaccagtcacaccagccattc

SEQ ID NO: 135 - DNA, 40 bp, synthetic construct
  misc_feature 1..40: note = Description of Artificial Sequence: Synthetic primer
  source 1..40: mol_type = other DNA
taatacgactcactatagggagctacaaccattgagggct

SEQ ID NO: 136 - DNA, 42 bp, synthetic construct
  misc_feature 1..42: note = Description of Artificial Sequence: Synthetic primer
  source 1..42: mol_type = other DNA
taatacgactcactatagggagcatccaaaagtactcggtgt

SEQ ID NO: 137 - DNA, 40 bp, synthetic construct
  misc_feature 1..40: note = Description of Artificial Sequence: Synthetic primer
  source 1..40: mol_type = other DNA
taatacgactcactatagggttctcgacctgtcagctgtt

SEQ ID NO: 138 - DNA, 40 bp, synthetic construct
  misc_feature 1..40: note = Description of Artificial Sequence: Synthetic primer
  source 1..40: mol_type = other DNA
taatacgactcactatagggcgtcagcatccaaacgcaat

SEQ ID NO: 139 - DNA, 40 bp, synthetic construct
  misc_feature 1..40: note = Description of Artificial Sequence: Synthetic primer
  source 1..40: mol_type = other DNA
taatacgactcactataggggtgacgctggaatatcccgt

SEQ ID NO: 140 - DNA, 40 bp, synthetic construct
  misc_feature 1..40: note = Description of Artificial Sequence: Synthetic primer
  source 1..40: mol_type = other DNA
taatacgactcactatagggcactcggcgacagtattccc

SEQ ID NO: 141 - DNA, 40 bp, synthetic construct
  misc_feature 1..40: note = Description of Artificial Sequence: Synthetic primer
  source 1..40: mol_type = other DNA
taatacgactcactatagggaatggggcaagagtccatct

SEQ ID NO: 142 - DNA, 40 bp, synthetic construct
  misc_feature 1..40: note = Description of Artificial Sequence: Synthetic primer
  source 1..40: mol_type = other DNA
taatacgactcactatagggacagacacacttgccagtca

SEQ ID NO: 143 - DNA, 40 bp, synthetic construct
  misc_feature 1..40: note = Description of Artificial Sequence: Synthetic primer
  source 1..40: mol_type = other DNA
taatacgactcactatagggagctttgcatccccatcact

SEQ ID NO: 144 - DNA, 43 bp, synthetic construct
  misc_feature 1..43: note = Description of Artificial Sequence: Synthetic primer
  source 1..43: mol_type = other DNA
taatacgactcactataggggtacaacatttacttgctgtcca

SEQ ID NO: 145 - DNA, 45 bp, synthetic construct
  misc_feature 1..45: note = Description of Artificial Sequence: Synthetic primer
  source 1..45: mol_type = other DNA
taatacgactcactatagggaacagttctgattggattttgctca

SEQ ID NO: 146 - DNA, 42 bp, synthetic construct
  misc_feature 1..42: note = Description of Artificial Sequence: Synthetic primer
  source 1..42: mol_type = other DNA
taatacgactcactatagggcatgtgtagtgcagacagaaca

SEQ ID NO: 147 - DNA, 43 bp, synthetic construct
  misc_feature 1..43: note = Description of Artificial Sequence: Synthetic primer
  source 1..43: mol_type = other DNA
taatacgactcactatagggggttttggataagagctgtgtca

SEQ ID NO: 148 - DNA, 44 bp, synthetic construct
  misc_feature 1..44: note = Description of Artificial Sequence: Synthetic primer
  source 1..44: mol_type = other DNA
taatacgactcactataggggtccagactttactcgtccgtgtc

SEQ ID NO: 149 - DNA, 40 bp, synthetic construct
  misc_feature 1..40: note = Description of Artificial Sequence: Synthetic primer
  source 1..40: mol_type = other DNA
taatacgactcactatagggctttcccgccgttttgtgaa

SEQ ID NO: 150 - DNA, 40 bp, synthetic construct
  misc_feature 1..40: note = Description of Artificial Sequence: Synthetic primer
  source 1..40: mol_type = other DNA
taatacgactcactatagggccagtatggggttgtgggac

SEQ ID NO: 151 - DNA, 40 bp, synthetic construct
  misc_feature 1..40: note = Description of Artificial Sequence: Synthetic primer
  source 1..40: mol_type = other DNA
taatacgactcactatagggagagggcaagcgcagtaata

SEQ ID NO: 152 - DNA, 40 bp, synthetic construct
  misc_feature 1..40: note = Description of Artificial Sequence: Synthetic primer
  source 1..40: mol_type = other DNA
taatacgactcactatagggaccgaaaccggctaaactga

SEQ ID NO: 153 - DNA, 41 bp, synthetic construct
  misc_feature 1..41: note = Description of Artificial Sequence: Synthetic primer
  source 1..41: mol_type = other DNA
taatacgactcactatagggtcaaagcgctgctttcctcgc

SEQ ID NO: 154 - DNA, 19 bp, synthetic construct
  misc_feature 1..19: note = Description of Artificial Sequence: Synthetic primer
  source 1..19: mol_type = other DNA
cttttggccattactgccg

SEQ ID NO: 155 - DNA, 24 bp, synthetic construct
  misc_feature 1..24: note = Description of Artificial Sequence: Synthetic primer
  source 1..24: mol_type = other DNA
gctctcagttcctggtatttttct

SEQ ID NO: 156 - DNA, 22 bp, synthetic construct
  misc_feature 1..22: note = Description of Artificial Sequence: Synthetic primer
  source 1..22: mol_type = other DNA
tcgttactgcacttgaaagcgt

SEQ ID NO: 157 - DNA, 21 bp, synthetic construct
  misc_feature 1..21: note = Description of Artificial Sequence: Synthetic primer
  source 1..21: mol_type = other DNA
ccatgaaactgggaagatgaa

SEQ ID NO: 158 - DNA, 19 bp, synthetic construct
  misc_feature 1..19: note = Description of Artificial Sequence: Synthetic primer
  source 1..19: mol_type = other DNA
gaagctctccagcagctca

SEQ ID NO: 159 - DNA, 21 bp, synthetic construct
  misc_feature 1..21: note = Description of Artificial Sequence: Synthetic primer
  source 1..21: mol_type = other DNA
gctgtcagcacattcttttcc

SEQ ID NO: 160 - DNA, 20 bp, synthetic construct
  misc_feature 1..20: note = Description of Artificial Sequence: Synthetic primer
  source 1..20: mol_type = other DNA
actgctcctcaccaatcgtc

SEQ ID NO: 161 - DNA, 26 bp, synthetic construct
  misc_feature 1..26: note = Description of Artificial Sequence: Synthetic primer
  source 1..26: mol_type = other DNA
tttatataatcggaaggaaccttttt

SEQ ID NO: 162 - DNA, 21 bp, synthetic construct
  misc_feature 1..21: note = Description of Artificial Sequence: Synthetic primer
  source 1..21: mol_type = other DNA
ggcaaaatgcttagatgcaga

SEQ ID NO: 163 - DNA, 20 bp, synthetic construct
  misc_feature 1..20: note = Description of Artificial Sequence: Synthetic primer
  source 1..20: mol_type = other DNA
tagccttgtgcaatgcttgt

SEQ ID NO: 164 - DNA, 24 bp, synthetic construct
  misc_feature 1..24: note = Description of Artificial Sequence: Synthetic primer
  source 1..24: mol_type = other DNA
cacttctggtaccaaatgatcaac

SEQ ID NO: 165 - DNA, 20 bp, synthetic construct
  misc_feature 1..20: note = Description of Artificial Sequence: Synthetic primer
  source 1..20: mol_type = other DNA
gcggcaaactttttgagtgt

SEQ ID NO: 166 - DNA, 20 bp, synthetic construct
  misc_feature 1..20: note = Description of Artificial Sequence: Synthetic primer
  source 1..20: mol_type = other DNA
cgcgtgatgaggatctgatt

SEQ ID NO: 167 - DNA, 21 bp, synthetic construct
  misc_feature 1..21: note = Description of Artificial Sequence: Synthetic primer
  source 1..21: mol_type = other DNA
aaaattaagagcgggcagact

SEQ ID NO: 168 - DNA, 22 bp, synthetic construct
  misc_feature 1..22: note = Description of Artificial Sequence: Synthetic primer
  source 1..22: mol_type = other DNA
aaagcacttgattgagaattgc

SEQ ID NO: 169 - DNA, 20 bp, synthetic construct
  misc_feature 1..20: note = Description of Artificial Sequence: Synthetic primer
  source 1..20: mol_type = other DNA
cagtttcccaagcttcaagg

SEQ ID NO: 170 - DNA, 20 bp, synthetic construct
  misc_feature 1..20: note = Description of Artificial Sequence: Synthetic primer
  source 1..20: mol_type = other DNA
aatggttgcagcattgtgtt

SEQ ID NO: 171 - DNA, 20 bp, synthetic construct
  misc_feature 1..20: note = Description of Artificial Sequence: Synthetic primer
  source 1..20: mol_type = other DNA
gttacctggcaaccaccaac

SEQ ID NO: 172 - DNA, 22 bp, synthetic construct
  misc_feature 1..22: note = Description of Artificial Sequence: Synthetic primer
  source 1..22: mol_type = other DNA
acgttaacaaaggcgatgtttt

SEQ ID NO: 173 - DNA, 20 bp, synthetic construct
  misc_feature 1..20: note = Description of Artificial Sequence: Synthetic primer
  source 1..20: mol_type = other DNA
tgacaggactcatcagcacg

SEQ ID NO: 174 - DNA, 22 bp, synthetic construct
  misc_feature 1..22: note = Description of Artificial Sequence: Synthetic primer
  source 1..22: mol_type = other DNA
tgggaaaaataccaggaagcgt

SEQ ID NO: 175 - DNA, 20 bp, synthetic construct
  misc_feature 1..20: note = Description of Artificial Sequence: Synthetic primer
  source 1..20: mol_type = other DNA
agatcaatgagagcgaggcg

SEQ ID NO: 176 - DNA, 21 bp, synthetic construct
  misc_feature 1..21: note = Description of Artificial Sequence: Synthetic primer
  source 1..21: mol_type = other DNA
cggacagtaatgtctggatgg

SEQ ID NO: 177 - DNA, 20 bp, synthetic construct
  misc_feature 1..20: note = Description of Artificial Sequence: Synthetic primer
  source 1..20: mol_type = other DNA
tcatcatggccaacagaatg

SEQ ID NO: 178 - DNA, 23 bp, synthetic construct
  misc_feature 1..23: note = Description of Artificial Sequence: Synthetic primer
  source 1..23: mol_type = other DNA
tgactcaaccaatcaatcagcct

SEQ ID NO: 179 - DNA, 23 bp, synthetic construct
  misc_feature 1..23: note = Description of Artificial Sequence: Synthetic primer
  source 1..23: mol_type = other DNA
ttctgtcttttaatcagcaatcc

SEQ ID NO: 180 - DNA, 23 bp, synthetic construct
  misc_feature 1..23: note = Description of Artificial Sequence: Synthetic primer
  source 1..23: mol_type = other DNA
tgaagcttgtacctttcatttcc

SEQ ID NO: 181 - DNA, 24 bp, synthetic construct
  misc_feature 1..24: note = Description of Artificial Sequence: Synthetic primer
  source 1..24: mol_type = other DNA
tatcagtgatgttctgcagtggtc

SEQ ID NO: 182 - DNA, 22 bp, synthetic construct
  misc_feature 1..22: note = Description of Artificial Sequence: Synthetic primer
  source 1..22: mol_type = other DNA
caggaagaaataatggcaaaaa

SEQ ID NO: 183 - DNA, 21 bp, synthetic construct
  misc_feature 1..21: note = Description of Artificial Sequence: Synthetic primer
  source 1..21: mol_type = other DNA
catttccaccagctgtctgat

SEQ ID NO: 184 - DNA, 20 bp, synthetic construct
  misc_feature 1..20: note = Description of Artificial Sequence: Synthetic primer
  source 1..20: mol_type = other DNA
ccctgctgatcacacatgac

SEQ ID NO: 185 - DNA, 22 bp, synthetic construct
  misc_feature 1..22: note = Description of Artificial Sequence: Synthetic primer
  source 1..22: mol_type = other DNA
tgcactaaatctgtgccaagtc

SEQ ID NO: 186 - DNA, 21 bp, synthetic construct
  misc_feature 1..21: note = Description of Artificial Sequence: Synthetic primer
  source 1..21: mol_type = other DNA
tcctgtcagctgttttcatcc

SEQ ID NO: 187 - DNA, 20 bp, synthetic construct
  misc_feature 1..20: note = Description of Artificial Sequence: Synthetic primer
  source 1..20: mol_type = other DNA
tgcgaggaggacataaacaa

SEQ ID NO: 188 - DNA, 21 bp, synthetic construct
  misc_feature 1..21: note = Description of Artificial Sequence: Synthetic primer
  source 1..21: mol_type = other DNA
tgctgaattcaaaagccactt

SEQ ID NO: 189 - DNA, 20 bp, synthetic construct
  misc_feature 1..20: note = Description of Artificial Sequence: Synthetic primer
  source 1..20: mol_type = other DNA
gagggttaaacgtggcctta

SEQ ID NO: 190 - DNA, 21 bp, synthetic construct
  misc_feature 1..21: note = Description of Artificial Sequence: Synthetic primer
  source 1..21: mol_type = other DNA
gccagcctcaaagtttgttct

SEQ ID NO: 191 - DNA, 20 bp, synthetic construct
  misc_feature 1..20: note = Description of Artificial Sequence: Synthetic primer
  source 1..20: mol_type = other DNA
ggtgttgaaaggtgatgctg

SEQ ID NO: 192 - DNA, 20 bp, synthetic construct
  misc_feature 1..20: note = Description of Artificial Sequence: Synthetic primer
  source 1..20: mol_type = other DNA
tggaaacaacaacagcctga

SEQ ID NO: 193 - DNA, 23 bp, synthetic construct
  misc_feature 1..23: note = Description of Artificial Sequence: Synthetic primer
  source 1..23: mol_type = other DNA
tgtttggttcagttacacgtttt

SEQ ID NO: 194 - DNA, 23 bp, synthetic construct
  misc_feature 1..23: note = Description of Artificial Sequence: Synthetic primer
  source 1..23: mol_type = other DNA
tgtgattacacattcccacacat

SEQ ID NO: 195 - DNA, 20 bp, synthetic construct
  misc_feature 1..20: note = Description of Artificial Sequence: Synthetic primer
  source 1..20: mol_type = other DNA
gcttttgtttggtgatgtgc

SEQ ID NO: 196 - DNA, 22 bp, synthetic construct
  misc_feature 1..22: note = Description of Artificial Sequence: Synthetic primer
  source 1..22: mol_type = other DNA
tggtcagaataagcacgtttca

SEQ ID NO: 197 - DNA, 27 bp, synthetic construct
  misc_feature 1..27: note = Description of Artificial Sequence: Synthetic primer
  source 1..27: mol_type = other DNA
tctaaacaatttttaaggtaaaccaaa

SEQ ID NO: 198 - DNA, 24 bp, synthetic construct
  misc_feature 1..24: note = Description of Artificial Sequence: Synthetic primer
  source 1..24: mol_type = other DNA
aatagtctctggtctgctgttaaa

SEQ ID NO: 199 - DNA, 23 bp, synthetic construct
  misc_feature 1..23: note = Description of Artificial Sequence: Synthetic primer
  source 1..23: mol_type = other DNA
aagcagcgagctctcataataaa

SEQ ID NO: 200 - DNA, 22 bp, synthetic construct
  misc_feature 1..22: note = Description of Artificial Sequence: Synthetic primer
  source 1..22: mol_type = other DNA
ggagcagatgaggttaagtcct

SEQ ID NO: 201 - DNA, 21 bp, synthetic construct
  misc_feature 1..21: note = Description of Artificial Sequence: Synthetic primer
  source 1..21: mol_type = other DNA
ccacaactccatactgggaaa

SEQ ID NO: 202 - DNA, 20 bp, synthetic construct
  misc_feature 1..20: note = Description of Artificial Sequence: Synthetic primer
  source 1..20: mol_type = other DNA
gtgcaggaaatgagcacaga

SEQ ID NO: 203 - DNA, 19 bp, synthetic construct
  misc_feature 1..19: note = Description of Artificial Sequence: Synthetic primer
  source 1..19: mol_type = other DNA
tagcaaagctctcaggccc

SEQ ID NO: 204 - DNA, 158 bp, Danio rerio; identical to SEQ ID NO: 13
  source 1..158: mol_type = unassigned DNA

SEQ ID NO: 205 - DNA, 125 bp, synthetic construct
  misc_feature 1..125: note = Description of Artificial Sequence: Synthetic polynucleotide
  source 1..125: mol_type = other DNA
tgaagcttgtacctttcatttaaattttgctgagctttattttctctagaattgccattgtgtttccattctagcaaatcagcatttttttttcagctgaaagaaaaataccatttactgagagc

SEQ ID NO: 206 - DNA, 124 bp, synthetic construct
  misc_feature 1..124: note = Description of Artificial Sequence: Synthetic polynucleotide
  source 1..124: mol_type = other DNA
tgaagcttgtacctttcatttcctttttgctgagcgggcggggatctagaattgcacggtggtttccattctagcaaatcagccggggggtttcagctaaagaaaaataccaggaactgagagc

SEQ ID NO: 207 - DNA, 126 bp, synthetic construct
  misc_feature 1..126: note = Description of Artificial Sequence: Synthetic polynucleotide
  source 1..126: mol_type = other DNA
attagcagatttaatttcatttcctttttgcattaatttattttctctagaattgccattgtgtttccattctagcaaatcagcatttttttttcagctgaaagaaaaataccaggaactgagagc

SEQ ID NO: 208 - DNA, 125 bp, synthetic construct
  misc_feature 1..125: note = Description of Artificial Sequence: Synthetic polynucleotide
  source 1..125: mol_type = other DNA
tgaagcttgtacctttcatttcctttttgctgagctttattttctctagaattgccattgtgtttccattccggcaaatcagcatttttttttcagctgaccgaaaaataccaggaactgagagc

SEQ ID NO: 209 - DNA, 158 bp, synthetic construct
  misc_feature 1..158: note = Description of Artificial Sequence: Synthetic polynucleotide
  source 1..158: mol_type = other DNA
ccatgaaactgggaagatgaaagcattagttgaattgttactggcaacatcttctctgtaatgccccctgtgacccatattgtctcgctctttaaattataaacagagctgtagatatccacatttaatgggggtgtttttgccattatttctaaatg

SEQ ID NO: 210 - DNA, 158 bp, synthetic construct
  misc_feature 1..158: note = Description of Artificial Sequence: Synthetic polynucleotide
  source 1..158: mol_type = other DNA
ccatgaaactgggaagatgaaagcattagttgaaggtggactggcaacatcttctctgtaatgccccctgtgacccataggtgatcgctctttcctttataaacagagctgtagatatccacaggaaatgggggtgtttttgccattatttcttcctg

SEQ ID NO: 211 - DNA, 158 bp, synthetic construct
  misc_feature 1..158: note = Description of Artificial Sequence: Synthetic polynucleotide
  source 1..158: mol_type = other DNA
ccatgaaactgggaagatgaaagcattagttgaattgttactggcaacatcttctctgtaatgccccctgattaacatattgtctcgctctttcctttataaacagagctgtagatatccacaggaaatgggggtgtttttgccattatttcttcctg

SEQ ID NO: 212 - DNA, 158 bp, synthetic construct
  misc_feature 1..158: note = Description of Artificial Sequence: Synthetic polynucleotide
  source 1..158: mol_type = other DNA
ccatgaaactgggaatctgaaagcattagttgaattgttactggcaacatcttctctgtaatgccccctgtgacccatattgtctcgctctttcctttataaacagagaggtagatatccacaggaaatgggggactttttgccattatttcttcctg

SEQ ID NO: 213 - DNA, 74 bp, synthetic construct
  misc_feature 1..74: note = Description of Artificial Sequence: Synthetic primer
  source 1..74: mol_type = other DNA
tgaagcttgtacctttcatttcctttttgctgagctttattttctctagaattgccattgtgtttccattctag

SEQ ID NO: 214 - DNA, 74 bp, synthetic construct
  misc_feature 1..74: note = Description of Artificial Sequence: Synthetic primer
  source 1..74: mol_type = other DNA
tgaagcttgtacctttcatttaaattttgctgagctttattttctctagaattgccattgtgtttccattctag

SEQ ID NO: 215 - DNA, 74 bp, synthetic construct
  misc_feature 1..74: note = Description of Artificial Sequence: Synthetic primer
  source 1..74: mol_type = other DNA
tgaagcttgtacctttcatttcctttttgctgagcgggcggggatctagaattgcacggtggtttccattctag

SEQ ID NO: 216 - DNA, 74 bp, synthetic construct
  misc_feature 1..74: note = Description of Artificial Sequence: Synthetic primer
  source 1..74: mol_type = other DNA
attagcagatttaatttcatttcctttttgcattaatttattttctctagaattgccattgtgtttccattcta

SEQ ID NO: 217 - DNA, 74 bp, synthetic construct; identical to SEQ ID NO: 213
  misc_feature 1..74: note = Description of Artificial Sequence: Synthetic primer
  source 1..74: mol_type = other DNA

SEQ ID NO: 218 - DNA, 91 bp, synthetic construct
  misc_feature 1..91: note = Description of Artificial Sequence: Synthetic primer
  source 1..91: mol_type = other DNA
ccatgaaactgggaagatgaaagcattagttgaattgttactggcaacatcttctctgtaatgccccctgtgacccatattgtctcgctct

SEQ ID NO: 219 - DNA, 91 bp, synthetic construct; identical to SEQ ID NO: 218
  misc_feature 1..91: note = Description of Artificial Sequence: Synthetic primer
  source 1..91: mol_type = other DNA

SEQ ID NO: 220 - DNA, 91 bp, synthetic construct
  misc_feature 1..91: note = Description of Artificial Sequence: Synthetic primer
  source 1..91: mol_type = other DNA
ccatgaaactgggaagatgaaagcattagttgaaggtggactggcaacatcttctctgtaatgccccctgtgacccataggtgatcgctct

SEQ ID NO: 221 - DNA, 91 bp, synthetic construct
  misc_feature 1..91: note = Description of Artificial Sequence: Synthetic primer
  source 1..91: mol_type = other DNA
ccatgaaactgggaagatgaaagcattagttgaattgttactggcaacatcttctctgtaatgccccctgattaacatattgtctcgctct

SEQ ID NO: 222 - DNA, 91 bp, synthetic construct
  misc_feature 1..91: note = Description of Artificial Sequence: Synthetic primer
  source 1..91: mol_type = other DNA
ccatgaaactgggaatctgaaagcattagttgaattgttactggcaacatcttctctgtaatgccccctgtgacccatattgtctcgctct

SEQ ID NO: 223 - DNA, 75 bp, synthetic construct
  misc_feature 1..75: note = Description of Artificial Sequence: Synthetic primer
  source 1..75: mol_type = other DNA
gctctcagttcctggtatttttctttcagctgaaaaaaaaatgctgatttgctagaatggaaacacaatggcaat

SEQ ID NO: 224 - DNA, 75 bp, synthetic construct
  misc_feature 1..75: note = Description of Artificial Sequence: Synthetic primer
  source 1..75: mol_type = other DNA
gctctcagtaaatggtatttttctttcagctgaaaaaaaaatgctgatttgctagaatggaaacacaatggcaat

SEQ ID NO: 225 - DNA, 75 bp, synthetic construct
  misc_feature 1..75: note = Description of Artificial Sequence: Synthetic primer
  source 1..75: mol_type = other DNA
gctctcagttcctggtatttttctttcagctgaaaccccccggctgatttgctagaatggaaaccaccgtgcaat

SEQ ID NO: 226 - DNA, 75 bp, synthetic construct; identical to SEQ ID NO: 223
  misc_feature 1..75: note = Description of Artificial Sequence: Synthetic primer
  source 1..75: mol_type = other DNA

SEQ ID NO: 227 - DNA, 75 bp, synthetic construct
  misc_feature 1..75: note = Description of Artificial Sequence: Synthetic primer
  source 1..75: mol_type = other DNA
gctctcagttcctggtatttttcggtcagctgaaaaaaaaatgctgatttgccggaatggaaacacaatggcaat

SEQ ID NO: 228 - DNA, 91 bp, synthetic construct
  misc_feature 1..91: note = Description of Artificial Sequence: Synthetic primer
  source 1..91: mol_type = other DNA
caggaagaaataatggcaaaaacacccccatttcctgtggatatctacagctctgtttataaaggaaagagcgagacaatatgggtcacag

SEQ ID NO: 229 - DNA, 91 bp, synthetic construct
  misc_feature 1..91: note = Description of Artificial Sequence: Synthetic primer
  source 1..91: mol_type = other DNA
catttagaaataatggcaaaaacacccccattaaatgtggatatctacagctctgtttataatttaaagagcgagacaatatgggtcacag

SEQ ID NO: 230 - DNA, 91 bp, synthetic construct
  misc_feature 1..91: note = Description of Artificial Sequence: Synthetic primer
  source 1..91: mol_type = other DNA
caggaagtttattaggcaaaaacacccccatttcctgtggatatctacagctctgtttataaaggaaagagcgatcacctatgggtcacag

SEQ ID NO: 231 - DNA, 91 bp, synthetic construct
  misc_feature 1..91: note = Description of Artificial Sequence: Synthetic primer
  source 1..91: mol_type = other DNA
caggaagaaataatggcaaaaacacccccatttcctgtggatatctacagctctgtttataaaggaaagagcgagacaatatgttaatcag

SEQ ID NO: 232 - DNA, 91 bp, synthetic construct
  misc_feature 1..91: note = Description of Artificial Sequence: Synthetic primer
  source 1..91: mol_type = other DNA
caggaagaaataatggcaaaaagtcccccatttcctgtggatatctacctctctgtttataaaggaaagagcgagacaatatgggtcacag

SEQ ID NO: 233 - DNA, 29 bp, synthetic construct
  misc_feature 1..29: note = Description of Artificial Sequence: Synthetic primer
  source 1..29: mol_type = other DNA
cgggatccatggcaatggtagtcagcacg

SEQ ID NO: 234 - DNA, 33 bp, synthetic construct
  misc_feature 1..33: note = Description of Artificial Sequence: Synthetic primer
  source 1..33: mol_type = other DNA
tttatgaagcttgtacctttcatttcctttttg

SEQ ID NO: 235 - DNA, 34 bp, synthetic construct
  misc_feature 1..34: note = Description of Artificial Sequence: Synthetic primer
  source 1..34: mol_type = other DNA
tttaattagcagatttaatttcatttcctttttg

SEQ ID NO: 236 - DNA, 30 bp, synthetic construct
  misc_feature 1..30: note = Description of Artificial Sequence: Synthetic primer
  source 1..30: mol_type = other DNA
ttcatttcctttttgctgagctttattttc

SEQ ID NO: 237 - DNA, 30 bp, synthetic construct
  misc_feature 1..30: note = Description of Artificial Sequence: Synthetic primer
  source 1..30: mol_type = other DNA
ttcatttcctttttgcattaatttattttc

SEQ ID NO: 238 - DNA, 44 bp, synthetic construct
  misc_feature 1..44: note = Description of Artificial Sequence: Synthetic primer
  source 1..44: mol_type = other DNA
gtaatgccccctgtgacccatattgtctcgctctttcctttata

SEQ ID NO: 239 - DNA, 44 bp, synthetic construct
  misc_feature 1..44: note = Description of Artificial Sequence: Synthetic primer
  source 1..44: mol_type = other DNA
gtaatgccccctgattaacatattgtctcgctctttcctttata

SEQ ID NO: 240 - DNA, 31 bp, synthetic construct
  misc_feature 1..31: note = Description of Artificial Sequence: Synthetic primer
  source 1..31: mol_type = other DNA
ccggaattccggttgaattgccatatatggc

SEQ ID NO: 241 - DNA, 33 bp, synthetic construct
  misc_feature 1..33: note = Description of Artificial Sequence: Synthetic primer
  source 1..33: mol_type = other DNA
caaaaaggaaatgaaaggtacaagcttcataaa

SEQ ID NO: 242 - DNA, 34 bp, synthetic construct
  misc_feature 1..34: note = Description of Artificial Sequence: Synthetic primer
  source 1..34: mol_type = other DNA
caaaaaggaaatgaaattaaatctgctaattaaa

SEQ ID NO: 243 - DNA, 30 bp, synthetic construct
  misc_feature 1..30: note = Description of Artificial Sequence: Synthetic primer
  source 1..30: mol_type = other DNA
gaaaataaagctcagcaaaaaggaaatgaa

SEQ ID NO: 244 - DNA, 30 bp, synthetic construct
  misc_feature 1..30: note = Description of Artificial Sequence: Synthetic primer
  source 1..30: mol_type = other DNA
gaaaataaattaatgcaaaaaggaaatgaa

SEQ ID NO: 245 - DNA, 44 bp, synthetic construct
  misc_feature 1..44: note = Description of Artificial Sequence: Synthetic primer
  source 1..44: mol_type = other DNA
tataaaggaaagagcgagacaatatgggtcacagggggcattac

SEQ ID NO: 246 - DNA, 44 bp, synthetic construct
  misc_feature 1..44: note = Description of Artificial Sequence: Synthetic primer
  source 1..44: mol_type = other DNA
tataaaggaaagagcgagacaatatgttaatcagggggcattac